SEQUENCE LISTING (114 sequences)

SEQ ID NO: 1 - DNA, 7855 bp, Artificial sequence
  vector E425
tcgacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattatttacccaccactttggtaatttcgcctttcacgtaatgcacaaattcttccagctgatccgcacggctggccagacgatcttcttcctggcccagatacgcctgacgcgcttccagaatcaccggctgatactgcgccggcagacgttccatggcccaatccgccgccacatctttcggcgcaattttgccggtcaccgcgctataccaaatacggctcagggtcagcaccacgttgcgttcatcacccgcccaatccggcgggctgttccacagggtcagggtttcgttcagcgcttcaaacaggtcctgttccggcaccggatcaaacagttcttccgctgccggaccaaccagcgccacgctatgttcacgcgctttggtcagcagaatggccagatcgatatcaatggtcgccggttcaaaaatgccggccagaatatcgttacgctgccattcgccaaactgcagctcacgtttcgccggataacgccacggaataatgtcatcatgcaccacgatggtcacttccaccgcacgcagaatttcgctttcacccgggctcgcgctggtttccagcagatcgttaatcagcgcacgacgggtggtttcatccagacgcacggtcacggtcaccagcagatcaatatcgctatgcggtttcaggccgccatccaccgcgctgccatacagatgcacggccagcagggtcggttccagatgacgttcaatcacgcccaccacttcagacagctgggtgctcacttccgcaatcaccgcttcacgcataatgttcagtttggttttcggacggctgccctgctgggtcacatcgttgctgctccacagcatcaggcaacggcccaccgcataacgcgcctgctgtttgctcgcacgcggcatgctctgcacgccaaaaaagcaatcatagcacgccataaacaccgccaccgcaccattaccgccgctacgttcggtcagggtacggctccaattacggctacgcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgagtgaaggccgtcaaggcctattgctcgatgagacgtcttaattaactggcctcatgggccttcacttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggcgcgcctgaataacttcgtatagcatacattatagcaatttatcgaaaaagcctgaactcaccgcgacatccgtggagaaattcctcatcgaaaaattcgactccgtgtccgatctcatgcagctgtccgagggcgaggagagtagagcattctcattcgatgtgggcgggagaggctacgtgctgagagtgaactcttgtgccgacggcttctacaaggaccgatacgtctaccggcattttgcttccgccgctctgcctattccagaagtcctggacattggggagtttagcgagtccctcacttactgtattagccggcgagcccagggagtgacactccaggatctgcctgaaactgaactgcctgctgtgctccagcctgtcgctgaggcaatggatgctattgctgctgccgatctgagtcagactagcggattcggcccatttggaccccagggcattggccagtacacaacatggcgagacttcatctgtgctatcgccgatcctcacgtgtaccattggcagactgtgatggacgatactgtgtctgcttctgtggcacaggcactcgacgaactcatgctgtgggctgaggactgtcctgaagtgagacatctggtccatgccgattttggctccaacaatgtgctcaccgataacgggagaatcactgccgtgatcgactggagcgaggcaatgtttggcgattcccagtacgaagtggccaacatcttcttttggcggccttggctggcttgtatggaacagcagacccggtactttgaacggcgccaccctgagctggctgggagtcctagactgagagcctacatgctccgaattggcctggatcagctctaccagtcactggtggatggcaatttcgacgatgctgcttgggcacaggggcgctgtgatgctattgtccgatccggcgctggaactgtggggagaacacagatcgctaggagatccgctgctgtctggaccgatggatgtgtggaagtgctggccgatagtggaaaccggaggccttcaacccgaccccgggcaaaggagtaatgaccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatcccgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacacaagctggcggccgctctcggaaggtctctatgttgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgagttcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtatctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgcctagcttcctgctgaacatcaaaggcaagaaaacatctgttgtcaaagacagcatccttgaacaaggacaattaacagttaacaaataaaaacgcaaaagaaaatgccgattatggtgcactctcagtacaatctggtctctcaggcgccctgaccagcggcgtgcataccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaaggtgggacccgtggggtgcgaatagaagttcctattccgaagttcctattctctagaaagtataggaacttcgggccacatggaattaattcagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcaaagctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgacc

SEQ ID NO: 2 - DNA, 1704 bp, Artificial sequence
  PCR product
catggcgagggcaccttcacctccgacctgtccaaacaaatggaagaagaagccgtccggctgttcatcgaatggctgaaaaatggcggcccttcctctggcgcccctcctccttctnnnnnnnnctggttcgacccctggggccagggaaccctggtcaccgtgtcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcataccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaaggtgggacccgtggggtgcgaatagaagttcctattccgaagttcctattctctagaaagtataggaacttcgggccacatggaattaattcagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcaaagctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgacctcgacggggtctgacgctcagtggaacgaaaactc

SEQ ID NO: 3 - DNA, 1630 bp, Artificial sequence
  fragment derived from P163
ggatcccgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactgctcgacgatctgatcaagagacaggataaggagccgccaccatggagtttgggctgagctggctttttcttgtggctattttaaaaggtgtccagtgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncacagtggtagtactccactgtctgggtgtacaaaaacctccctgcacgcctctctaacctcacaattctgtggcggccgcgccgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggtgagtacaggaggtggagagtacgcgtaacacttaagcgtctctccaa

SEQ ID NO: 4 - DNA, 2423 bp, Artificial sequence
  vector V610 digested with KpnI and BsaI
ggtacctcttaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgcccgtctctccaagtgcaaagggacaggaggtttttgttaagggctgtatcactgtggagacc

SEQ ID NO: 5 - DNA, 8514 bp, Artificial sequence
  acceptor vector E501
cagactgccccggggcagttaggagatccgggggccgccgggaactgccgctggccccccaccgccccaaggatctcccggtccccgcccggcgtgctgacgtcacggcgctgccccagggtgtgctgggcaggtcgcggggagcgctgggaaatggagtccattagcagaagtggcccttggccacttccaggagtcgctgtgccccgatgcacactgggaagtccgcagctccgaggcgcccagtggaaatcgccagatgagggcctcctccggggaatgctgggaaatggagtctacaggccggaggggtgccccacggcatactaggaagtgtgtagcaccgggtaaaggggatgaatagcagactgccccggggcagttaggagatccgggggccgccgggaactgccgctggccccccaccgccccaaggatctcccggtccccgcccggcgtgctgacgtcacggcgctgccccagggtgtgctgggcaggtcgcggggagcgctgggaaatggagtccattagcagaagtggcccttggccacttccaggagtcgctgtgccccgatgcacactgggaagtccgcagctccgaggcgcccagtggaaatcgccagatgagggcctcctccggggaatgctgggaaatggagtctacaggccggaggggtgccccacggcatactaggaagtgtgtagcaccgggtaaaggggatgaatagcagactgccccggggcagttaggagatccgggggccgccgggaactgccgctggccccccaccgccccaaggatctcccggtccccgcccggcgtgctgacgtcacggcgctgccccagggtgtgctgggcaggtcgcggggagcgctgggaaatggagtccattagcagaagtggcccttggccacttccaggagtcgctgtgccccgatgcacactgggaagtccgcagctccgaggcgcccagtggaaatcgccagatgagggcctcctccggggaatgctgggaaatggagtctacaggccggaggggtgccccacggcatactaggaagtgtgtagcaccgggtaaaggggatgaatagcagactgccccggggcagttaggagatccctcatgggccttcctttcactgcactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgaggagccgccaccatggacatgagggtccccgctcagctcctggggctcctgctactctggctccgaggtaaggatggagaacactaggaatttactcagccagtgtgctcagtactgactggaacttcagggaagttctctgataacatgattaatagtaagaatatttgtttttatgtttccaatctcaggtgccagatgtgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaagcggaccgtggccgctcccagcgtgttcatcttcccccccagcgacgagcagcttaagagcggtaccgctagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgctaatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcacgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgattcgagttaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgcctgaataacttcgtatagcatacattatagcaatttatcgaaaaagcctgaactcaccgcgacatccgtggagaaattcctcatcgaaaaattcgactccgtgtccgatctcatgcagctgtccgagggcgaggagagtagagcattctcattcgatgtgggcgggagaggctacgtgctgagagtgaactcttgtgccgacggcttctacaaggaccgatacgtctaccggcattttgcttccgccgctctgcctattccagaagtcctggacattggggagtttagcgagtccctcacttactgtattagccggcgagcccagggagtgacactccaggatctgcctgaaactgaactgcctgctgtgctccagcctgtcgctgaggcaatggatgctattgctgctgccgatctgagtcagactagcggattcggcccatttggaccccagggcattggccagtacacaacatggcgagacttcatctgtgctatcgccgatcctcacgtgtaccattggcagactgtgatggacgatactgtgtctgcttctgtggcacaggcactcgacgaactcatgctgtgggctgaggactgtcctgaagtgagacatctggtccatgccgattttggctccaacaatgtgctcaccgataacgggagaatcactgccgtgatcgactggagcgaggcaatgtttggcgattcccagtacgaagtggccaacatcttcttttggcggccttggctggcttgtatggaacagcagacccggtactttgaacggcgccaccctgagctggctgggagtcctagactgagagcctacatgctccgaattggcctggatcagctctaccagtcactggtggatggcaatttcgacgatgctgcttgggcacaggggcgctgtgatgctattgtccgatccggcgctggaactgtggggagaacacagatcgctaggagatccgctgctgtctggaccgatggatgtgtggaagtgctggccgatagtggaaaccggaggccttcaacccgaccccgggcaaaggagtaatgaccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatcccgcgttgacattgattattgactagcctcggattctcgaggctaggctagtgagacgatacgtctcatcgacgatccgggggccgccgggaactgccgctggccccccaccgccccaaggatctcccggtccccgcccggcgtgctgacgtcacggcgctgccccagggtgtgctgggcaggtcgcggggagcgctgggaaatggagtccattagcagaagtggcccttggccacttccaggagtcgctgtgccccgatgcacactgggaagtccgcagctccgaggcgcccagtggaaatcgccagatgagggcctcctccggggaatgctgggaaatggagtctacaggccggaggggtgccccacggcatactaggaagtgtgtagcaccgggtaaaggggatgaatagcagactgccccggggcagttaggagatccgggggccgccgggaactgccgctggccccccaccgccccaaggatctcccggtccccgcccggcgtgctgacgtcacggcgctgccccagggtgtgctgggcaggtcgcggggagcgctgggaaatggagtccattagcagaagtggcccttggccacttccaggagtcgctgtgccccgatgcacactgggaagtccgcagctccgaggcgcccagtggaaatcgccagatgagggcctcctccggggaatgctgggaaatggagtctacaggccggaggggtgccccacggcatactaggaagtgtgtagcaccgggtaaaggggatgaatag

SEQ ID NO: 6 - DNA, 7593 bp, Artificial sequence
  vector comprising V503 expression cassette containing coding sequence for CRE recombinase
gacggatcgggagatctcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccgccaccatgggcagcaacaacgccctccagaccatcatcaacgcccgcctgcccggcgaagagggcctgtggcagatccatctccaggacggcaagatcagcgccatcgacgcccagagcggcgtgatgcccatcaccgagaacagcctggacgccgagcagggcctggtgatcccccccttcgtggaaccccacatccacctggacaccacccagacagccggccagcccaactggaaccagtccggcaccctgttcgagggcatcgagagatgggccgagcggaaggccctgctgacccacgacgacgtgaagcagcgggcctggcagaccctgaagtggcagatcgccaacggcatccagcacgtgcggacacacgtggacgtgtccgatgccaccctgaccgccctgaaggccatgctggaagtgaagcaggaagtggccccctggatcgacctccagatcgtggccttcccacaggaaggcatcctgagctaccccaacggcgaagccctgctggaagaggccctgagactgggcgccgacgtggtcggagccatcccccacttcgagttcacccgcgagtacggcgtggaaagcctgcacaagaccttcgccctggcccagaagtacgaccggctgatcgatgtgcattgcgacgagatcgacgacgagcagagcagattcgtggaaaccgtggccgccctggctcaccacgagggaatgggagccagagtgaccgccagccacaccaccgccatgcacagctacaacggcgcctacacctcccggctgttccggctgctgaagatgagcggcatcaacttcgtggccaaccccctggtgaacatccatctgcaaggcagattcgacacctaccccaagcggagaggcatcaccagagtgaaagagatgctggaatccggcatcaacgtgtgcttcggccacgacgatgtgttcgacccctggtatcccctgggcaccgccaacatgctccaggtgctgcacatgggcctgcacgtgtgccagctgatgggctacggccagatcaacgacggcctgaacctgatcacccaccacagcgcccggaccctgaacctgcaagactacggcattgccgccggaaacagcgccaacctgatcatcctgcccgccgagaacggcttcgacgccctgcggagacaggtgccagtgcggtatagcgtgcggggaggcaaagtgatcgcctccacccagcccgcccagaccaccgtgtacctggaacagcccgaggccatcgactacaagagatgagcggccctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgctgtgaaatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagccatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcacgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 7 - protein, 39 aa, Heloderma suspectum
  misc_feature: Exendin-4
HGEGRFTSDLSKQMEEEAVRLFIEWLKNGGPSSGAPPPS

SEQ ID NO: 8 - protein, 30 aa, Homo sapiens
  misc_feature: GLP-1
HAEGTFTSDVSSYLEGQAAKEFIAWLVKGR

SEQ ID NO: 9 - protein, 34 aa, Homo sapiens
  misc_feature: GLP-2
HADGSFSDEMNTILDNLAARDFINWLIQTKITDR

SEQ ID NO: 10 - protein, 29 aa, Homo sapiens
  misc_feature: Glucagon
HSQGTFTSDYSKYLDSRRAQDRVQWLMNT

SEQ ID NO: 11 - protein, 38 aa, Homo sapiens
  misc_feature: PACAP-38
HSDGIFTDSYSRYRKQMAVKKYLAAVLGKRYKQRVKNK

SEQ ID NO: 12 - protein, 115 aa, Homo sapiens
  misc_feature: parathyroid hormone
MIPAKDMAKVMIVMLAICFLTKSDGKSVKKRSVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 13 - DNA, 12 bp, Artificial sequence
  RSS spacer
atacagacctta

SEQ ID NO: 14 - DNA, 12 bp, Artificial sequence
  RSS spacer
ctacagactgga

SEQ ID NO: 15 - DNA, 12 bp, Artificial sequence
  RSS spacer
ctccagggctga

SEQ ID NO: 16 - DNA, 12 bp, Artificial sequence
  RSS spacer
gtacagaccaat

SEQ ID NO: 17 - DNA, 23 bp, Artificial sequence
  RSS spacer
gtagtactccactgtctggctgt

SEQ ID NO: 18 - DNA, 23 bp, Artificial sequence
  RSS spacer
ttgcaaccacatcctgagtgtgt

SEQ ID NO: 19 - DNA, 23 bp, Artificial sequence
  RSS spacer
acggagataaaggaggaagcagg

SEQ ID NO: 20 - DNA, 23 bp, Artificial sequence
  RSS spacer
gccgggccccgcggcccggcggc

SEQ ID NO: 21 - DNA, 5986 bp, Artificial sequence
  vector V525 expressing GLP1 receptor
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgcgaattcgcccttgatggcccagtcctgaactccccgccatggccggcgcccccggcccgctgcgccttgcgctgctgctgctcgggatggtgggcagggccggcccccgcccccagggtgccactgtgtccctctgggagacggtgcagaaatggcgagaataccgacgccagtgccagcgctccctgactgaggatccacctcctgccacagacttgttctgcaaccggaccttcgatgaatacgcctgctggccagatggggagccaggctcgttcgtgaatgtcagctgcccctggtacctgccctgggccagcagtgtgccgcagggccacgtgtaccggttctgcacagctgaaggcctctggctgcagaaggacaactccagcctgccctggagggacttgtcggagtgcgaggagtccaagcgaggggagagaagctccccggaggagcagctcctgttcctctacatcatctacacggtgggctacgcactctccttctctgctctggttatcgcctctgcgatcctcctcggcttcagacacctgcactgcaccaggaactacatccacctgaacctgtttgcatccttcatcctgcgagcattgtccgtcttcatcaaggacgcagccctgaagtggatgtatagcacagccgcccagcagcaccagtgggatgggctcctctcctaccaggactctctgagctgccgcctggtgtttctgctcatgcagtactgtgtggcggccaattactactggctcttggtggagggcgtgtacctgtacacactgctggccttctcggtcttctctgagcaatggatcttcaggctctacgtgagcataggctggggtgttcccctgctgtttgttgtcccctggggcattgtcaagtacctctatgaggacgagggctgctggaccaggaactccaacatgaactactggctcattatccggctgcccattctctttggcattggggtgaacttcctcatctttgttcgggtcatctgcatcgtggtatccaaactgaaggccaatctcatgtgcaagacagacatcaaatgcagacttgccaagtccacgctgacactcatccccctgctggggactcatgaggtcatctttgcctttgtgatggacgagcacgcccgggggaccctgcgcttcatcaagctgtttacagagctctccttcacctccttccaggggctgatggtggccatattatactgctttgtcaacaatgaggtccagctggaatttcggaagagctgggagcgctggcggcttgagcacttgcacatccagagggacagcagcatgaagcccctcaagtgtcccaccagcagcctgagcagtggagccacggcgggcagcagcatgtacacagccacttgccaggcctcctgcagctgagactccagcgcctgccctccctggggtccttgctgcaggcctggtggccaatccaggtgggagagacactcaagggcgaattcagatctggtaccgatatcaagcttgtcgactctagattgcggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcctcgactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatatt

SEQ ID NO: 22 - DNA, 6723 bp, Artificial sequence
  vector E518 with cAMP response element and minimal promoter driving CD19
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcgctagcgcaccagacagtgacgtcagctgccagatcccatggccgtcatactgtgacgtctttcagacaccccattgacgtcaatgggagaacagatctggcctcggcggccaagcttagacactagagggtatataatggaagctcgacttccagcttggcaatccggtactactagcgccgccaccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccaggtgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 23 - DNA, 475 bp, Artificial sequence
  ITS007-V105 (VH sequence through the end of the JH)
caggtcacactgagagagtccggccctgccctggtgaaacccacccagaccctgaccctgacatgcaccttcagcggcttcagcctgagcaccagcgggatgtgcgtgtcctggattcgacagccccctggcaaggccctggaatggctggccctgattgactgggacgacgacaagtactacagcaccagcctgaaaacccggctgaccatcagcaaggacaccagcaagaaccaggtggtgctgaccatgaccaacatggaccccgtggacaccgccacgtattactgtgcacggcatggcgagggcaccttcacctccgacctgtccaaacaaatggaagaagaagccgtccggctgttcatcgaatggctgaaaaatggcggcccttcctctggcgcccctcctccttcttcgctgcaggatgatgcttttgatatctggggccaagggacaatggtcaccgtgtcctcag

SEQ ID NO: 24 - protein, 158 aa, Artificial sequence
  ITS007-V105 (VH sequence through the end of the JH)
QVTLRESGPALVKPTQTLTLTCTFSGFSLSTSGMCVSWIRQPPGKALEWLALIDWDDDKYYSTSLKTRLTISKDTSKNQVVLTMTNMDPVDTATYYCARHGEGTFTSDLSKQMEEEAVRLFIEWLKNGGPSSGAPPPSSLQDDAFDIWGQGTMVTVSS

SEQ ID NO: 25 - DNA, 472 bp, Artificial sequence
  ITS007-V107 (VH sequence through the end of the JH)
gaagtgcagctggtggaaagcggcggaggcctggtgcagcccggtcgaagcctgagactgagctgcgccgccagcggcttcacctttgacgactacgccatgcactgggtccgccaggcccctggcaagggactggaatgggtgtccggcatcagctggaacagcggcagcatcggctacgccgacagcgtgaagggccggttcaccatcagccgggacaacgccaagaacagcctgtacctgcagatgaacagcctgcgggccgaggacaccgccttgtattactgtgcgagagatgagcggcatggcgagggcaccttcacctccgacctgtccaaacaaatggaagaagaagccgtccggctgttcatcgaatggctgaaaaatggcggcccttcctctggcgcccctcctccttctgtggctgcttttgatatctggggccaagggacaatggtcaccgtgtcctcag

SEQ ID NO: 26 - protein, 157 aa, Artificial sequence
  ITS007-V107 (VH sequence through the end of the JH)
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCARDERHGEGTFTSDLSKQMEEEAVRLFIEWLKNGGPSSGAPPPSVAAFDIWGQGTMVTVSS

SEQ ID NO: 27 - DNA, 41 bp, Artificial sequence
  primer
gagagtttggatcccaactttcttgtccaccttggtgttgc

SEQ ID NO: 28 - DNA, 50 bp, Artificial sequence
  primer
gagagatttggtctctatgtcgatctgatcaagagacaggataaggagcc

SEQ ID NO: 29 - DNA, 41 bp, Artificial sequence
  primer
gagagagattggtctcgcctgagttccacgacaccgtcacc

SEQ ID NO: 30 - DNA, 58 bp, Artificial sequence
  primer
ctacgaggtctcactgtgnnkcatggcgagggcaccttcacctccgacctgtccaaac

SEQ ID NO: 31 - DNA, 55 bp, Artificial sequence
  primer
ttaagaggtaccctcgagcagacatgataagatacattgatgagtttggacaaac

SEQ ID NO: 32 - protein, 62 aa, Artificial sequence
  ITS007-V105 exendin junction
CARHGEGTFTSDLSKQMEEEAVRLFIEWLKNGGPSSGAPPPSSLQDDAFDIWGQGTMVTVSS

SEQ ID NO: 33 - protein, 62 aa, Artificial sequence
  ITS007-V107 exendin junction
CARDERHGEGTFTSDLSKQMEEEAVRLFIEWLKNGGPSSGAPPPSVAAFDIWGQGTMVTVSS

SEQ ID NO: 34 - protein, 37 aa, Homo sapiens
  misc_feature: Oxyntomodulin
HSQGTFTSDYSKYLDSRRAQDFVQWLMNTKRNRNNIA

SEQ ID NO: 35 - DNA, 87 bp, Artificial sequence
  sequence encoding glucagon
cattcacagggcacattcaccagtgactacagcaagtatctggactccaggcgtgcccaagattttgtgcagtggttgatgaatacc

SEQ ID NO: 36 - DNA, 111 bp, Artificial sequence
  sequence encoding oxyntomodulin
cattcacagggcacattcaccagtgactacagcaagtatctggactccaggcgtgcccaagattttgtgcagtggttgatgaataccaagaggaacaggaataacattgcc

SEQ ID NO: 37 - protein, 42 aa, Artificial sequence
  ITS011-V36 glucagon junction
CTTDLSRRRHSQGTFTSDYSKYLDSRRAQDFVQWLMNTLPMP

SEQ ID NO: 38 - protein, 41 aa, Artificial sequence
  ITS011-V66 glucagon junction
CTTDPVLKHSQGTFTSDYSKYLDSRRAQDFVQWLMNTLAPV

SEQ ID NO: 39 - protein, 43 aa, Artificial sequence
  ITS011-V69 glucagon junction
CARAESAGRHSQGTFTSDYSKYLDSRRAQDFVQWLMNTTSMVR

SEQ ID NO: 40 - protein, 45 aa, Artificial sequence
  ITS011-V23 oxyntomodulin junction
CASNRHSQGTFTSDYSKYLDSRRAQDFVQWLMNTKRNRNNIAMLL

SEQ ID NO: 41 - protein, 50 aa, Artificial sequence
  ITS011-V74 oxyntomodulin junction
CARDKGIKHSQGTFTSDYSKYLDSRRAQDFVQWLMNTKRNRNNIASKQVR

SEQ ID NO: 42 - protein, 58 aa, Artificial sequence
  ITS011-V100 oxyntomodulin junction
CARDLSDLSHSVLLRHSQGTFTSDYSKYLDSRRAQDFVQWLMNTKRNRNNIARPLVLS

SEQ ID NO: 43 - protein, 28 aa, Homo sapiens
  misc_feature: Vasoactive intestinal peptide
HSDAVFTDNYTRLRKQMAVKKYLNSILN

SEQ ID NO: 44 - protein, 27 aa, Homo sapiens
  misc_feature: PACAP-27
HSDGIFTDSYSRYRKQMAVKKYLAAVL

SEQ ID NO: 45 - protein, 35 aa, Heloderma suspectum
  misc_feature: helodermin
HSDAIFTEEYSKLLAKLALQKYLASILGSRTSPPP

SEQ ID NO: 46 - protein, 27 aa, Homo sapiens
  misc_feature: Peptide histidine methionine
HADGVFTSDFSKLLGQLSAKKYLESLM

SEQ ID NO: 47 - protein, 27 aa, Homo sapiens
  misc_feature: secretin
HSDGTFTSELSRLREGARLQRLLQGLV

SEQ ID NO: 48 - protein, 44 aa, Homo sapiens
  misc_feature: Growth hormone releasing factor
YADAIFTNSYRKVLGQLSARKLLQDIMSRQQGESNQERGARARL

SEQ ID NO: 49 - protein, 42 aa, Homo sapiens
  misc_feature: Gastric inhibitory peptide
YAEGTFISDYSIAMDKIHQQDFVNWLLAQKGKKNDWKHNITQ

SEQ ID NO: 50 - DNA, 1224 bp, Artificial sequence
  ITS007-V125
atggagtttgggctgagctggctttttcttgtggctattttaaaaggtgtccagtgtgaagtgcagctggtggaaagcggcggaggcctggtgcagcccggtcgaagcctgagactgagctgcgccgccagcggcttcacctttgacgactacgccatgcactgggtccgccaggcccctggcaagggactggaatgggtgtccggcatcagctggaacagcggcagcatcggctacgccgacagcgtgaagggccggttcaccatcagccgggacaacgccaagaacagcctgtacctgcagatgaacagcctgcgggccgaggacaccgccttgtattactgtgcgagagatgagcggcatggcgagggcaccttcacctccgacctgtccaaacaaatggaagaagaagccgtccggctgttcatcgaatggctgaaaaatggcggcccttcctctggcgcccctcctccttctgtggctgcttttgatatctggggccaagggacaatggtcaccgtgtcctcagagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcaaa

SEQ ID NO: 51 - protein, 408 aa, Artificial sequence
  ITS007-V125
MEFGLSWLFLVAILKGVQCEVQLVESGGGLVQPGRSLRLSCAASGFTFDDYAMHWVRQAPGKGLEWVSGISWNSGSIGYADSVKGRFTISRDNAKNSLYLQMNSLRAEDTALYYCARDERHGEGTFTSDLSKQMEEEAVRLFIEWLKNGGPSSGAPPPSVAAFDIWGQGTMVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - DNA, 4917 bp, Artificial sequence
  ITS007-V248
tcgagttaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctacgcgccgaccgagtccactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactgctcgacgatctgatcaagagacaggataaggagccgccaccatggagtttgggctgagctggctttttcttgtggctattttaaaaggtgtccagtgtgaagtgcagctggtggaaagcggcggaggcctggtgcagcccggtcgaagcctgagactgagctgcgccgccagcggcttcacctttgacgactacgccatgcactgggtccgccaggcccctggcaagggactggaatgggtgtccggcatcagctggaacagcggcagcatcggctacgccgacagcgtgaagggccggttcaccatcagccgggacaacgccaagaacagcctgtacctgcagatgaacagcctgcgggccgaggacaccgccttgtattactgtagcagaaagaagcggcatggcgagggcaccttcacctccgacctgtccaaacaaatggaagaagaagccgtccggctgttcatcgaatggctgaaaaatggcggcccttcctctggcgcccctcctccttctgtggctgcttttgatatctggggccaagggacaatggtcaccgtgtcctcagagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcaaatctggcgaagtgcagctggtggaaagcggcggaggcctggtgcagcccggtcgaagcctgagactgagctgcgccgccagcggcttcacctttgacgactacgccatgcactgggtccgccaggcccctggcaagggactggaatgggtgtccggcatcagctggaacagcggcagcatcggctacgccgacagcgtgaagggccggttcaccatcagccgggacaacgccaagaacagcctgtacctgcagatgaacagcctgcgggccgaggacaccgccttgtattactgtagcagaaagaagcggcatggcgagggcaccttcacctccgacctgtccaaacaaatggaagaagaagccgtccggctgttcatcgaatggctgaaaaatggcggcccttcctctggcgcccctcctccttctgtggctgcttttgatatctggggccaagggacaatggtcaccgtgtcctcataggttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgacc

SEQ ID NO: 53 - DNA, 471 bp, Artificial sequence
  ITS007-V126 (from VH to the end of JH)
gaagtgcagctggtggaaagcggcggaggcctggtgcagcccggtcgaagcctgagactgagctgcgccgccagcggcttcacctttgacgactacgccatgcactgggtccgccaggcccctggcaagggactggaatgggtgtccggcatcagctggaacagcggcagcatcggctacgccgacagcgtgaagggccggttcaccatcagccgggacaacgccaagaacagcctgtacctgcagatgaacagcctgcgggccgaggacaccgccttgtattactgtgcgaaggatgagcggcatggcgagggcaccttcacctccgacctgtccaaacaaatggaagaagaagccgtccggctgttcatcgaatggctgaaaaatggcggcccttcctctggcgcccctcctccttctgtggctgcttttgatatctggggccaagggacaatggtcaccgtgtcctca

SEQ ID NO: 54 - protein, 157 aa, Artificial sequence
  ITS007-V126 (from VH to the end of JH)
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDERHGEGTFTSDLSKQMEEEAVRLFIEWLKNGGPSSGAPPPSVAAFDIWGQGTMVTVSS

SEQ ID NO: 55 - DNA, 471 bp, Artificial sequence
  ITS007-V127 (from VH to the end of JH)
gaagtgcagctggtggaaagcggcggaggcctggtgcagcccggtcgaagcctgagactgagctgcgccgccagcggcttcacctttgacgactacgccatgcactgggtccgccaggcccctggcaagggactggaatgggtgtccggcatcagctggaacagcggcagcatcggctacgccgacagcgtgaagggccggttcaccatcagccgggacaacgccaagaacagcctgtacctgcagatgaacagcctgcgggccgaggacaccgccttgtattactgtgcgagagatagacggcatggcgagggcaccttcacctccgacctgtccaaacaaatggaagaagaagccgtccggctgttcatcgaatggctgaaaaatggcggcccttcctctggcgcccctcctccttctgtggctgcttttgatatctggggccaagggacaatggtcaccgtgtcctca

SEQ ID NO: 56 - protein, 157 aa, Artificial sequence
  ITS007-V127 (from VH to the end of JH)
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCARDRRHGEGTFTSDLSKQMEEEAVRLFIEWLKNGGPSSGAPPPSVAAFDIWGQGTMVTVSS

SEQ ID NO: 57 - DNA, 471 bp, Artificial sequence
  ITS007-V128 (from VH to the end of JH)
gaagtgcagctggtggaaagcggcggaggcctggtgcagcccggtcgaagcctgagactgagctgcgccgccagcggcttcacctttgacgactacgccatgcactgggtccgccaggcccctggcaagggactggaatgggtgtccggcatcagctggaacagcggcagcatcggctacgccgacagcgtgaagggccggttcaccatcagccgggacaacgccaagaacagcctgtacctgcagatgaacagcctgcgggccgaggacaccgccttgtattactgtgcgagagatgagaagcatggcgagggcaccttcacctccgacctgtccaaacaaatggaagaagaagccgtccggctgttcatcgaatggctgaaaaatggcggcccttcctctggcgcccctcctccttctgtggctgcttttgatatctggggccaagggacaatggtcaccgtgtcctca

SEQ ID NO: 58 - protein, 157 aa, artificial sequence
  ITS007-V128 (from VH to the end of JH)
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCARDEKHGEGTFTSDLSKQMEEEAVRLFIEWLKNGGPSSGAPPPSVAAFDIWGQGTMVTVSS

SEQ ID NO: 59 - DNA, 471 bp, Artificial sequence
  ITS007-V130 (from VH to the end of JH)
gaagtgcagctggtggaaagcggcggaggcctggtgcagcccggtcgaagcctgagactgagctgcgccgccagcggcttcacctttgacgactacgccatgcactgggtccgccaggcccctggcaagggactggaatgggtgtccggcatcagctggaacagcggcagcatcggctacgccgacagcgtgaagggccggttcaccatcagccgggacaacgccaagaacagcctgtacctgcagatgaacagcctgcgggccgaggacaccgccttgtattactgtagcagaaagaagcggcatggcgagggcaccttcacctccgacctgtccaaacaaatggaagaagaagccgtccggctgttcatcgaatggctgaaaaatggcggcccttcctctggcgcccctcctccttctgtggctgcttttgatatctggggccaagggacaatggtcaccgtgtcctca

SEQ ID NO: 60 - protein, 157 aa, Artificial sequence
  ITS007-V130 (from VH to the end of JH)
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCSRKKRHGEGTFTSDLSKQMEEEAVRLFIEWLKNGGPSSGAPPPSVAAFDIWGQGTMVTVSS

SEQ ID NO: 61 - DNA, 477 bp, Artificial sequence
  ITS007-V206 (from VH to the end of JH)
gaagtgcagctggtggaaagcggcggaggcctggtgcagcccggtcgaagcctgagactgagctgcgccgccagcggcttcacctttgacgactacgccatgcactgggtccgccaggcccctggcaagggactggaatgggtgtccggcatcagctggaacagcggcagcatcggctacgccgacagcgtgaagggccggttcaccatcagccgggacaacgccaagaacagcctgtacctgcagatgaacagcctgcgggccgaggacaccgccttgtattactgtgcgagagatgagcggcatggcgagggcaccttcacctccgacctgtccaaacaaatggaagaagaagccgtccggctgttcatcgaatggctgaaaaatggcggcccttcggaggcctctggcgcccctcctccttctgtggctgcttttgatatctggggccaagggacaatggtcaccgtgtcctca

SEQ ID NO: 62 - protein, 159 aa, Artificial sequence
  ITS007-V206 (from VH to the end of JH)
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCARDERHGEGTFTSDLSKQMEEEAVRLFIEWLKNGGPSEASGAPPPSVAAFDIWGQGTMVTVSS

SEQ ID NO: 63 - DNA, 480 bp, Artificial sequence
  ITS007-V209 (from VH to the end of JH)
gaagtgcagctggtggaaagcggcggaggcctggtgcagcccggtcgaagcctgagactgagctgcgccgccagcggcttcacctttgacgactacgccatgcactgggtccgccaggcccctggcaagggactggaatgggtgtccggcatcagctggaacagcggcagcatcggctacgccgacagcgtgaagggccggttcaccatcagccgggacaacgccaagaacagcctgtacctgcagatgaacagcctgcgggccgaggacaccgccttgtattactgtgcgagagatgagcggcatggcgagggcaccttcacctccgacctgtccaaacaaatggaagaagaagccgtccggctgttcatcgaatggctgaaaaatggcggcccttccgcggagtcctctggcgcccctcctccttctgtggctgcttttgatatctggggccaagggacaatggtcaccgtgtcctca

SEQ ID NO: 64 - protein, 160 aa, Artificial sequence
  ITS007-V209 (from VH to the end of JH)
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCARDERHGEGTFTSDLSKQMEEEAVRLFIEWLKNGGPSAESSGAPPPSVAAFDIWGQGTMVTVSS

SEQ ID NO: 65 - DNA, 477 bp, Artificial sequence
  ITS007-V211 (from VH to the end of JH)
gaagtgcagctggtggaaagcggcggaggcctggtgcagcccggtcgaagcctgagactgagctgcgccgccagcggcttcacctttgacgactacgccatgcactgggtccgccaggcccctggcaagggactggaatgggtgtccggcatcagctggaacagcggcagcatcggctacgccgacagcgtgaagggccggttcaccatcagccgggacaacgccaagaacagcctgtacctgcagatgaacagcctgcgggccgaggacaccgccttgtattactgtgcgagagatgagcggcatggcgagggcaccttcacctccgacctgtccaaacaaatggaagaagaagccgtccggctgttcatcgaatggctgaaaaatggcggcccttccgagtcctctggcgcccctcctccttctgtggctgcttttgatatctggggccaagggacaatggtcaccgtgtcctca

SEQ ID NO: 66 - protein, 159 aa, Artificial sequence
  ITS007-V211 (from VH to the end of JH)
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCARDERHGEGTFTSDLSKQMEEEAVRLFIEWLKNGGPSESSGAPPPSVAAFDIWGQGTMVTVSS

SEQ ID NO: 67 - DNA, 474 bp, Artificial sequence
  ITS007-V212 (from VH to the end of JH)
gaagtgcagctggtggaaagcggcggaggcctggtgcagcccggtcgaagcctgagactgagctgcgccgccagcggcttcacctttgacgactacgccatgcactgggtccgccaggcccctggcaagggactggaatgggtgtccggcatcagctggaacagcggcagcatcggctacgccgacagcgtgaagggccggttcaccatcagccgggacaacgccaagaacagcctgtacctgcagatgaacagcctgcgggccgaggacaccgccttgtattactgtgcgaaggatgagtggcggcatggcgagggcaccttcacctccgacctgtccaaacaaatggaagaagaagccgtccggctgttcatcgaatggctgaaaaatggcggcccttcctctggcgcccctcctccttctgtggctgcttttgatatctggggccaagggacaatggtcaccgtgtcctca

SEQ ID NO: 68 - protein, 158 aa, Artificial sequence
  ITS007-V212 (from VH to the end of JH)
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDEWRHGEGTFTSDLSKQMEEEAVRLFIEWLKNGGPSSGAPPPSVAAFDIWGQGTMVTVSS

SEQ ID NO: 69 - DNA, 592 bp, Artificial sequence
  ITS007-V152 (tet) tetracycline inducible CMV promoter
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtccctatcagtgatagagatctatataagtccctatcagtgatagagagagctct

SEQ ID NO: 70 - DNA, 477 bp, Artificial sequence
  ITS007-V241 (from VH to the end of JH)
gaagtgcagctggtggaaagcggcggaggcctggtgcagcccggtcgaagcctgagactgagctgcgccgccagcggcttcacctttgacgactacgccatgcactgggtccgccaggcccctggcaagggactggaatgggtgtccggcatcagctggaacagcggcagcatcggctacgccgacagcgtgaagggccggttcaccatcagccgggacaacgccaagaacagcctgtacctgcagatgaacagcctgcgggccgaggacaccgccttgtattactgtgcgaaggatgagcggcatggcgagggcaccttcacctccgacctgtccaaacaaatggaagaagaagccgtccggctgttcatcgaatggctgaaaaatggcggcccttcggaggcctctggcgcccctcctccttctgtggctgcttttgatatctggggccaagggacaatggtcaccgtgtcctca

SEQ ID NO: 71 - protein, 159 aa, Artificial sequence
  ITS007-V241 (from VH to the end of JH)
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDERHGEGTFTSDLSKQMEEEAVRLFIEWLKNGGPSEASGAPPPSVAAFDIWGQGTMVTVSS

SEQ ID NO: 72 - DNA, 480 bp, Artificial sequence
  ITS007-V242 (from VH to the end of JH)
gaagtgcagctggtggaaagcggcggaggcctggtgcagcccggtcgaagcctgagactgagctgcgccgccagcggcttcacctttgacgactacgccatgcactgggtccgccaggcccctggcaagggactggaatgggtgtccggcatcagctggaacagcggcagcatcggctacgccgacagcgtgaagggccggttcaccatcagccgggacaacgccaagaacagcctgtacctgcagatgaacagcctgcgggccgaggacaccgccttgtattactgtgcgaaggatgagcggcatggcgagggcaccttcacctccgacctgtccaaacaaatggaagaagaagccgtccggctgttcatcgaatggctgaaaaatggcggcccttccgcggagtcctctggcgcccctcctccttctgtggctgcttttgatatctggggccaagggacaatggtcaccgtgtcctca

SEQ ID NO: 73 - protein, 160 aa, Artificial sequence
  ITS007-V242 (from VH to the end of JH)
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDERHGEGTFTSDLSKQMEEEAVRLFIEWLKNGGPSAESSGAPPPSVAAFDIWGQGTMVTVSS

SEQ ID NO: 74 - DNA, 477 bp, Artificial sequence
  ITS007-V243 (from VH to the end of JH)
gaagtgcagctggtggaaagcggcggaggcctggtgcagcccggtcgaagcctgagactgagctgcgccgccagcggcttcacctttgacgactacgccatgcactgggtccgccaggcccctggcaagggactggaatgggtgtccggcatcagctggaacagcggcagcatcggctacgccgacagcgtgaagggccggttcaccatcagccgggacaacgccaagaacagcctgtacctgcagatgaacagcctgcgggccgaggacaccgccttgtattactgtgcgaaggatgagcggcatggcgagggcaccttcacctccgacctgtccaaacaaatggaagaagaagccgtccggctgttcatcgaatggctgaaaaatggcggcccttccgagtcctctggcgcccctcctccttctgtggctgcttttgatatctggggccaagggacaatggtcaccgtgtcctca

SEQ ID NO: 75 - protein, 159 aa, Artificial sequence
  ITS007-V243 (from VH to the end of JH)
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDERHGEGTFTSDLSKQMEEEAVRLFIEWLKNGGPSESSGAPPPSVAAFDIWGQGTMVTVSS

SEQ ID NO: 76 - DNA, 477 bp, Artificial sequence
  ITS007-V244 (from VH to the end of JH)
gaagtgcagctggtggaaagcggcggaggcctggtgcagcccggtcgaagcctgagactgagctgcgccgccagcggcttcacctttgacgactacgccatgcactgggtccgccaggcccctggcaagggactggaatgggtgtccggcatcagctggaacagcggcagcatcggctacgccgacagcgtgaagggccggttcaccatcagccgggacaacgccaagaacagcctgtacctgcagatgaacagcctgcgggccgaggacaccgccttgtattactgtagcagaaagaagcggcatggcgagggcaccttcacctccgacctgtccaaacaaatggaagaagaagccgtccggctgttcatcgaatggctgaaaaatggcggcccttcggaggcctctggcgcccctcctccttctgtggctgcttttgatatctggggccaagggacaatggtcaccgtgtcctca

SEQ ID NO: 77 - protein, 159 aa, Artificial sequence
  ITS007-V244 (from VH to the end of JH)
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCSRKKRHGEGTFTSDLSKQMEEEAVRLFIEWLKNGGPSEASGAPPPSVAAFDIWGQGTMVTVSS

SEQ ID NO: 78 - DNA, 480 bp, Artificial sequence
  ITS007-V245 (from VH to the end of JH)
gaagtgcagctggtggaaagcggcggaggcctggtgcagcccggtcgaagcctgagactgagctgcgccgccagcggcttcacctttgacgactacgccatgcactgggtccgccaggcccctggcaagggactggaatgggtgtccggcatcagctggaacagcggcagcatcggctacgccgacagcgtgaagggccggttcaccatcagccgggacaacgccaagaacagcctgtacctgcagatgaacagcctgcgggccgaggacaccgccttgtattactgtagcagaaagaagcggcatggcgagggcaccttcacctccgacctgtccaaacaaatggaagaagaagccgtccggctgttcatcgaatggctgaaaaatggcggcccttccgcggagtcctctggcgcccctcctccttctgtggctgcttttgatatctggggccaagggacaatggtcaccgtgtcctca

SEQ ID NO: 79 - protein, 160 aa, Artificial sequence
  ITS007-V245 (from VH to the end of JH)
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCSRKKRHGEGTFTSDLSKQMEEEAVRLFIEWLKNGGPSAESSGAPPPSVAAFDIWGQGTMVTVSS

SEQ ID NO: 80 - DNA, 477 bp, Artificial sequence
  ITS007-V246 (from VH to the end of JH)
gaagtgcagctggtggaaagcggcggaggcctggtgcagcccggtcgaagcctgagactgagctgcgccgccagcggcttcacctttgacgactacgccatgcactgggtccgccaggcccctggcaagggactggaatgggtgtccggcatcagctggaacagcggcagcatcggctacgccgacagcgtgaagggccggttcaccatcagccgggacaacgccaagaacagcctgtacctgcagatgaacagcctgcgggccgaggacaccgccttgtattactgtagcagaaagaagcggcatggcgagggcaccttcacctccgacctgtccaaacaaatggaagaagaagccgtccggctgttcatcgaatggctgaaaaatggcggcccttccgagtcctctggcgcccctcctccttctgtggctgcttttgatatctggggccaagggacaatggtcaccgtgtcctca

SEQ ID NO: 81 - protein, 159 aa, Artificial sequence
  ITS007-V246 (from VH to the end of JH)
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCSRKKRHGEGTFTSDLSKQMEEEAVRLFIEWLKNGGPSESSGAPPPSVAAFDIWGQGTMVTVSS

SEQ ID NO: 82 - protein, 47 aa, Artificial sequence
  ITS007-V125 exendin junction
CARDERHGEGTFTSDLSKQMEEEAVRLFIEWLKNGGPSSGAPPPSVA

SEQ ID NO: 83 - protein, 47 aa, Artificial sequence
  ITS007-V126 exendin junction
CAKDERHGEGTFTSDLSKQMEEEAVRLFIEWLKNGGPSSGAPPPSVA

SEQ ID NO: 84 - protein, 47 aa, Artificial sequence
  ITS007-V127 exendin junction
CARDRRHGEGTFTSDLSKQMEEEAVRLFIEWLKNGGPSSGAPPPSVA

SEQ ID NO: 85 - protein, 47 aa, Artificial sequence
  ITS007-V128 exendin junction
CARDEKHGEGTFTSDLSKQMEEEAVRLFIEWLKNGGPSSGAPPPSVA

SEQ ID NO: 86 - protein, 47 aa, Artificial sequence
  ITS007-V130 exendin junction
CSRKKRHGEGTFTSDLSKQMEEEAVRLFIEWLKNGGPSSGAPPPSVA

SEQ ID NO: 87 - protein, 48 aa, Artificial sequence
  ITS007-V212 exendin junction
CAKDEWRHGEGTFTSDLSKQMEEEAVRLFIEWLKNGGPSSGAPPPSVA

SEQ ID NO: 88 - protein, 49 aa, Artificial sequence
  ITS007-V206 exendin junction
CARDERHGEGTFTSDLSKQMEEEAVRLFIEWLKNGGPSEASGAPPPSVA

SEQ ID NO: 89 - protein, 50 aa, Artificial sequence
  ITS007-V209 exendin junction
CARDERHGEGTFTSDLSKQMEEEAVRLFIEWLKNGGPSAESSGAPPPSVA

SEQ ID NO: 90 - protein, 49 aa, Artificial sequence
  ITS007-V211 exendin junction
CARDERHGEGTFTSDLSKQMEEEAVRLFIEWLKNGGPSESSGAPPPSVA

SEQ ID NO: 91 - protein, 49 aa, Artificial sequence
  ITS007-V241 exendin junction
CAKDERHGEGTFTSDLSKQMEEEAVRLFIEWLKNGGPSEASGAPPPSVA

SEQ ID NO: 92 - protein, 50 aa, Artificial sequence
  ITS007-V242 exendin junction
CAKDERHGEGTFTSDLSKQMEEEAVRLFIEWLKNGGPSAESSGAPPPSVA

SEQ ID NO: 93 - protein, 49 aa, Artificial sequence
  ITS007-V243 exendin junction
CAKDERHGEGTFTSDLSKQMEEEAVRLFIEWLKNGGPSESSGAPPPSVA

SEQ ID NO: 94 - protein, 49 aa, Artificial sequence
  ITS007-V244 exendin junction
CSRKKRHGEGTFTSDLSKQMEEEAVRLFIEWLKNGGPSEASGAPPPSVA

SEQ ID NO: 95 - protein, 50 aa, Artificial sequence
  ITS007-V245 exendin junction
CSRKKRHGEGTFTSDLSKQMEEEAVRLFIEWLKNGGPSAESSGAPPPSVA

SEQ ID NO: 96 - protein, 49 aa, Artificial sequence
  ITS007-V246 exendin junction
CSRKKRHGEGTFTSDLSKQMEEEAVRLFIEWLKNGGPSESSGAPPPSVA

SEQ ID NO: 97 - DNA, 465 bp, Artificial sequence
  ITS011-V36 (from VH to the end of JH)
gaagtgcagctggtggaaagcggcggaggcctggtgaaacctggcggcagcctgagactgagctgcgccgccagcggcttcaccttctcgaacgcctggatgagctgggtccgccaggcccctggcaagggactggaatgggtcggacggatcaagagcaagaccgacggcggcaccaccgactacgctgcccccgtgaagggccggttcaccatcagccgggacgacagcaagaacaccctgtacctgcagatgaacagcctgaaaaccgaggacaccgccgtgtattactgtaccacagatctgtccaggaggaggcattcacagggcacattcaccagtgactacagcaagtatctggactccaggcgtgcccaagattttgtgcagtggttgatgaataccctgcctatgcccgatgcttttgatatctggggccaagggacaatggtcaccgtgtcctca

SEQ ID NO: 98 - protein, 155 aa, Artificial sequence
  ITS011-V36 (from VH to the end of JH)
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTDLSRRRHSQGTFTSDYSKYLDSRRAQDFVQWLMNTLPMPDAFDIWGQGTMVTVSS

SEQ ID NO: 99 - DNA, 471 bp, Artificial sequence
  ITS011-V66 (from VH to the end of JH)
gaagtgcagctggtggaaagcggcggaggcctggtgaaacctggcggcagcctgagactgagctgcgccgccagcggcttcaccttctcgaacgcctggatgagctgggtccgccaggcccctggcaagggactggaatgggtcggacggatcaagagcaagaccgacggcggcaccaccgactacgctgcccccgtgaagggccggttcaccatcagccgggacgacagcaagaacaccctgtacctgcagatgaacagcctgaaaaccgaggacaccgccgtgtattactgtaccacagatcccgtgcttaagcattcacagggcacattcaccagtgactacagcaagtatctggactccaggcgtgcccaagattttgtgcagtggttgatgaataccctggcgcctgtaaactactactactacatggacgtctggggcaaagggaccacggtcaccgtgtcctca

SEQ ID NO: 100 - protein, 157 aa, Artificial sequence
  ITS011-V66 (from VH to the end of JH)
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTDPVLKHSQGTFTSDYSKYLDSRRAQDFVQWLMNTLAPVNYYYYMDVWGKGTTVTVSS

SEQ ID NO: 101 - DNA, 459 bp, Artificial sequence
  ITS011-V69 (from VH to the end of JH)
gaagtgcagctggtggaaagcggaggcggagtggttcgacctggcggaagcctgagactgtcttgcgccgccagcggcttcacctttgacgactacggcatgagctgggtccgccaggcccctggcaagggactggaatgggtgtccggcatcaactggaacggcggcagcaccggctacgccgacagcgtgaagggccggttcaccatcagccgggacaacgccaagaacagcctgtacctgcagatgaacagcctgcgggccgaggacaccgccttgtatcactgtgcgagagcggaaagcgcggggaggcattcacagggcacattcaccagtgactacagcaagtatctggactccaggcgtgcccaagattttgtgcagtggttgatgaataccacttcgatggtgcggtacttccagcactggggccagggcaccctggtcaccgtgtcctca

SEQ ID NO: 102 - protein, 153 aa, Artificial sequence
  ITS011-V69 (from VH to the end of JH)
EVQLVESGGGVVRPGGSLRLSCAASGFTFDDYGMSWVRQAPGKGLEWVSGINWNGGSTGYADSVKGRFTISRDNAKNSLYLQMNSLRAEDTALYHCARAESAGRHSQGTFTSDYSKYLDSRRAQDFVQWLMNTTSMVRYFQHWGQGTLVTVSS

SEQ ID NO: 103 - DNA, 477 bp, Artificial sequence
  ITS011-V23 (from VH to the end of JH)
caggtgcagctggtggaaagcggcggaggcgtggtgcagcccggcagaagcctgagactgagctgcgccgccagcggcttcaccttcagcagctacgccatgcactgggtccgccaggcccctggcaagggactggaatgggtggccgtgatcagctacgacggcagcaacaagtactacgccgacagcgtgaagggccggttcaccatctctcgagacaacagcaagaacaccctgtacctgcagatgaacagcctgcgggccgaggacaccgccgtgtattactgtgcgagtaataggcattcacagggcacattcaccagtgactacagcaagtatctggactccaggcgtgcccaagattttgtgcagtggttgatgaataccaagaggaacaggaataacattgccatgcttcttgactactactactacatggacgtctggggcaaagggaccacggtcaccgtgtcctca

SEQ ID NO: 104 - protein, 159 aa, Artificial sequence
  ITS011-V23 (from VH to the end of JH)
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCASNRHSQGTFTSDYSKYLDSRRAQDFVQWLMNTKRNRNNIAMLLDYYYYMDVWGKGTTVTVSS

SEQ ID NO: 105 - DNA, 483 bp, Artificial sequence
  ITS011-V74 (from VH to the end of JH)
gaagtgcagctggtggaaagcggaggcggagtggttcgacctggcggaagcctgagactgtcttgcgccgccagcggcttcacctttgacgactacggcatgagctgggtccgccaggcccctggcaagggactggaatgggtgtccggcatcaactggaacggcggcagcaccggctacgccgacagcgtgaagggccggttcaccatcagccgggacaacgccaagaacagcctgtacctgcagatgaacagcctgcgggccgaggacaccgccttgtatcactgtgcgagagataaagggattaagcattcacagggcacattcaccagtgactacagcaagtatctggactccaggcgtgcccaagattttgtgcagtggttgatgaataccaagaggaacaggaataacattgccagtaagcaggtgcgatggtacttcgatctctggggccgtggcaccctggtcactgtgtcctca

SEQ ID NO: 106 - protein, 161 aa, Artificial sequence
  ITS011-V74 (from VH to the end of JH)
EVQLVESGGGVVRPGGSLRLSCAASGFTFDDYGMSWVRQAPGKGLEWVSGINWNGGSTGYADSVKGRFTISRDNAKNSLYLQMNSLRAEDTALYHCARDKGIKHSQGTFTSDYSKYLDSRRAQDFVQWLMNTKRNRNNIASKQVRWYFDLWGRGTLVTVSS

SEQ ID NO: 107 - DNA, 510 bp, Artificial sequence
  ITS011-V100 (from VH to the end of JH)
caggtgcagctggtggaaagcggcggaggcgtggtgcagcccggcagaagcctgagactgagctgcgccgccagcggcttcaccttcagcagctacgccatgcactgggtccgccaggcccctggcaagggactggaatgggtggccgtgatcagctacgacggcagcaacaagtactacgccgacagcgtgaagggccggttcaccatctctcgagacaacagcaagaacaccctgtacctgcagatgaacagcctgcgggccgaggacaccgccgtgtattactgtgcgagagatctctcggatctctcgcacagtgtattactgaggcattcacagggcacattcaccagtgactacagcaagtatctggactccaggcgtgcccaagattttgtgcagtggttgatgaataccaagaggaacaggaataacattgcccggccgcttgtcctcagcgctgaatacttccagcactggggccagggcaccctggtcaccgtgtcctca

SEQ ID NO: 108 - protein, 170 aa, Artificial sequence
  ITS011-V100 (from VH to the end of JH)
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARDLSDLSHSVLLRHSQGTFTSDYSKYLDSRRAQDFVQWLMNTKRNRNNIARPLVLSAEYFQHWGQGTLVTVSS

SEQ ID NO: 109 - DNA, 477 bp, Artificial sequence
  ITS011-V165 (from VH to the end of JH)
caggtgcagctgcagcagtggggagccggcctgctgaagcccagcgagacactgagcctgacctgcgccgtgtacggcggcagcttcagcggctactactggtcctggatcagacagccccctggcaagggcctggaatggatcggcgagatcaaccacagcggcagcaccaactacaaccccagcctgaagtccagagtgaccatcagcgtggacacctcgaagaaccagttcagcctgaagctgagcagcgtgacagccgccgacaccgctgtgtattactgtgcgagaggccccatcctaaggccccggaggcattcacagggcacattcaccagtgactacagcaagtatctggactccaggcgtgcccaagattttgtgcagtggttgatgaataccaagaggaacaggaataacattgcccatagtattgtggactactggggccagggaaccctggtcaccgtgtcctca

SEQ ID NO: 110 - protein, 159 aa, Artificial sequence
  ITS011-V165 (from VH to the end of JH)
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGPILRPRRHSQGTFTSDYSKYLDSRRAQDFVQWLMNTKRNRNNIAHSIVDYWGQGTLVTVSS

SEQ ID NO: 111 - DNA, 468 bp, Artificial sequence
  ITS011-V171 (from VH to the end of JH)
caggtgcagctggtggaaagcggcggaggcgtggtgcagcccggcagaagcctgagactgagctgcgccgccagcggcttcaccttcagcagctacgccatgcactgggtccgccaggcccctggcaagggactggaatgggtggccgtgatcagctacgacggcagcaacaagtactacgccgacagcgtgaagggccggttcaccatctctcgagacaacagcaagaacaccctgtacctgcagatgaacagcctgcgggccgaggacaccgccgtgtattactgtgcgagagatcggttttcccgatccttttctaagcattcacagggcacattcaccagtgactacagcaagtatctggactccaggcgtgcccaagattttgtgcagtggttgatgaataccaagaggaacaggaataacattgctaactggggccagggcaccctggtcaccgtgtcctca

SEQ ID NO: 112 - protein, 156 aa, Artificial sequence
  ITS011-V171 (from VH to the end of JH)
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARDRFSRSFSKHSQGTFTSDYSKYLDSRRAQDFVQWLMNTKRNRNNIANWGQGTLVTVSS

SEQ ID NO: 113 - protein, 52 aa, Artificial sequence
  ITS011-V165 oxyntomodulin junction
CARGPILRPRRHSQGTFTSDYSKYLDSRRAQDFVQWLMNTKRNRNNIAHSIV

SEQ ID NO: 114 - protein, 50 aa, Artificial sequence
  ITS011-V171 oxyntomodulin junction
CARDRFSRSFSKHSQGTFTSDYSKYLDSRRAQDFVQWLMNTKRNRNNIAN

(19) United States
(12) Patent Application Publication
(10) Pub. No.: US 2016/0297883 A1
(43) Pub. Date: Oct. 13, 2016

(54) G-PROTEIN COUPLED RECEPTOR AGONISTS AND METHODS

(71) Applicant: Innovative Targeting Solutions, Inc., Burnaby (CA)

(72) Inventors: Michael Gallo, North Vancouver (CA); Jaspal Singh Kang, Surrey (CA); Craig Robin Pigott, Vancouver (CA); Abby YU Chen Lin, Surrey (CA)

(21) Appl. No.: 15/101,566

(22) PCT Filed: Dec. 4, 2014
(86) PCT No.: PCT/CA2014/051167

§ 371 Date: Jun. 3, 2016

Related U.S. Application Data
(60) Provisional application No. 62/043,874, filed on Aug. 29, 2014.
(60) Provisional application No. 61/911,829, filed on Dec. 4, 2013.

(51) Int. Cl. C07K 16/28; C12Q 1/68; C07K 14/605

(52) U.S. Cl. CPC C07K 16/2869; C07K 14/605; C12Q 1/6897; C07K 2319/75; C07K 2317/565; C07K 2317/51; C07K 2317/56; C07K 2317/526; A61K 38/00

(57) ABSTRACT

Peptide-grafted antibodies having agonist activity against a Class B GPCR and their uses, and methods for identifying same. Mammalian cell lines for high-throughput screening for agonists of a Class B GPCR are also provided that comprise a stably integrated recombinant nucleic acid construct comprising a reporter gene under control of a cAMP responsive promoter, the reporter gene encoding a cell surface expressed protein, and a stably integrated recombinant nucleic acid construct comprising a sequence encoding a Class B GPCR under control of a constitutive promoter.

### FIELD OF THE INVENTION

The present invention relates to the field of biologics and, in particular, to peptide-grafted antibodies capable of activating GPCRs and methods for identifying same.

### BACKGROUND OF THE INVENTION

G-protein coupled receptors (GPCRs) are a large family of transmembrane proteins that are involved in a wide variety of physiological processes. Class B GPCRs (the secretin receptor family) include a number of hormone receptors such as secretin receptors, vasoactive intestinal peptide receptors, calcitonin receptors and parathyroid hormone receptors.

The glucagon-like peptide 1 receptor (GLP1R) is a class B GPCR. The ligands appear to bind to GLP1R via a two step mechanism (Runge et al., 2008, J Biol. Chem., 283(17): 11340-11347; Underwood et al., 2010, J Biol. Chem., 285(1):723-730). The first step is binding to the amino terminus of the receptor via the carboxy terminus of the ligand. A second step of binding results in activation of the receptor and is mediated by the amino terminus of the ligand. The carboxy terminus of the ligand appears to be able to accommodate modification and in fact is active as a fusion protein such as GLP1-Fc. The amino terminus of GLP1 receptor ligands, on the other hand, appears to be significantly more sensitive to modification (Kieffer & Habener, (1999), Endocrine Reviews, 20(6), 876-913). The removal of the N-terminal histidine of the mature GLP1 peptide has been shown to result in a loss of binding and activity. The addition of one amino acid to the amino terminus of GLP1 also reduces activity. Single histidine to alanine substitution of the terminal histidine has shown this residue to be important for receptor binding. Similarly, the removal of the terminal histidine from Exendin also results in reduced activity. A two amino-acid deletion from the N-terminus of Exendin results in an antagonist even though the binding affinity is not significantly decreased.

Cross-linking studies using another class B GPCR, the parathyroid hormone receptor (PTHR), have shown that the N-terminal serine of the PTH ligand interacts directly with the receptor (Rolz et al., 1999, Biochemistry, 38(20):6397-6405). The PTHrp peptide has a N-terminal alanine and is also capable of activating the PTH receptor indicating that the side chain may not be directly involved in the contact with the receptor.

Peptide agonists of class B GPCRs, such as the GLP1R agonist Exendin-4, have potential as therapeutic agents. One drawback to the use of peptides as therapeutic reagents, however, is that they are generally unstable in vivo, i.e. their clearance rates from serum may be quite rapid. Fusion of the peptide to another larger molecule is one approach often adopted in order to stabilize the peptide in vivo, however conformational changes and/or other molecular forces that occur in peptide fusions may interfere with or negate the activity of the peptide. Each such peptide fusion must therefore be tested empirically in order to determine whether it has retained sufficient activity to be therapeutically useful.

International Patent Application No. PCT/US2004/041946 (WO 2005/060642) describes a method of producing rationally designed antibodies that comprise a peptide in one or more CDRs. A library of peptide-grafted antibodies that comprised GLP1 fused into the heavy chain CDR of a human tetanus toxoid specific antibody is described, but was not tested for the ability of the antibodies to bind GLP1R.

International Patent Application No. PCT/CA2013/050204 (WO 2013/134881) describes methods of generating fusion protein variants, including immunoglobulins comprising a peptide in one or more CDR. Immunoglobulins comprising GLP1 or exendin fused into the CDR of the immunoglobulin are described that are capable of binding to GLP1R.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The present invention relates generally to peptide-grafted antibodies having GPCR agonist activity and their uses, and methods for identifying same. In one aspect, the invention relates to a peptide-grafted antibody or antibody fragment comprising at least one peptide grafted into at least one CDR of the antibody or antibody fragment, the peptide comprising the sequence of a peptide agonist of a Class B GPCR or a functional fragment of the peptide agonist, wherein the peptide-grafted antibody has agonist activity against the Class B GPCR.

In another aspect, the invention relates to a peptide-grafted antibody or antibody fragment comprising at least one peptide grafted into at least one CDR of the antibody or antibody fragment, the peptide comprising the sequence of the exendin, GLP1, glucagon or oxyntomodulin peptide or a functional fragment thereof, wherein the peptide-grafted antibody has agonist activity against the GLP1 receptor, the glucagon receptor, or both.

In certain embodiments, in the peptide-grafted antibody or antibody fragment, the grafted peptide comprises a basic amino acid upstream and adjacent to the natural N-terminal amino acid of the peptide agonist.

In another aspect, the invention relates to a polynucleotide encoding a peptide-grafted antibody or antibody fragment as described above.

In another aspect, the invention relates to a mammalian cell line for high-throughput screening for agonists of a Class B GPCR, the cell line comprising: a first stably integrated recombinant nucleic acid construct comprising a reporter gene operably associated with a cAMP responsive promoter, the reporter gene encoding a cell-surface expressed protein, and a second stably integrated recombinant nucleic acid construct comprising a sequence encoding a Class B GPCR operably associated with a promoter.

In certain embodiments, the mammalian cell line is recombination competent.

In another aspect, the invention relates to a method for screening for peptide-grafted antibodies having agonist activity against a Class B GPCR, the method comprising: stably transfecting cells from a cell line as described above with a library of polynucleotides encoding peptide-grafted antibodies, wherein the peptide is a candidate peptide agonist of a Class B GPCR; culturing the cells under conditions that permit expression of the peptide-grafted antibodies and expression of the Class B GPCR, and screening the cells for expression of the cell-surface expressed protein.

In another aspect, the invention relates to a method for screening for peptide-grafted antibodies having agonist activity against a Class B GPCR, the method comprising: stably transfecting cells from a recombination competent cell line as described above with either (a) a nucleic acid sequence encoding a plurality of VH gene sequences, a candidate peptide agonist of the Class B GPCR and at least one JH gene sequence, and comprising one or more pairs of RSS sequences positioned to allow recombination of the nucleic acid sequence to form a polynucleotide encoding an antibody heavy chain comprising the peptide agonist, or (b) a nucleic acid sequence encoding a plurality of VL gene sequences, a candidate peptide agonist of the Class B GPCR and at least one JL gene sequence, and comprising one or more pairs of RSS sequences positioned to allow recombination of the nucleic acid sequence to form a polynucleotide encoding an antibody light chain comprising the peptide agonist; culturing the cells under conditions that permit V(D)J recombination, expression of the antibody heavy or light chain comprising the peptide agonist and expression of the Class B GPCR, and screening the cultured cells for expression of the cell-surface expressed protein.

In another aspect, the invention relates to a method for preparing a heavy-chain only peptide-grafted antibody having agonist activity against a Class B GPCR, the method comprising: stably transfecting cells from a recombination competent cell line as described above with a nucleic acid sequence encoding a plurality of VH gene sequences, a candidate peptide agonist of the Class B GPCR and at least one JH gene sequence, and comprising one or more pairs of RSS sequences positioned to allow recombination of the nucleic acid sequence to form a polynucleotide encoding an antibody heavy chain comprising the peptide agonist; culturing the cells under conditions that permit V(D)J recombination, expression of the antibody heavy chain comprising the peptide agonist and expression of the Class B GPCR; screening the cultured cells for expression of the cell-surface expressed protein; isolating a cell that expresses the cell-surface expressed protein, and isolating a polynucleotide encoding the peptide-grafted antibody having agonist activity against a Class B GPCR from the isolated cell.

### BRIEF DESCRIPTION OF THE DRAWINGS

These and other features of the invention will become more apparent in the following detailed description in which reference is made to the appended drawings.

FIG. 1 shows (A) a schematic diagram, and (B) the nucleic acid sequence [SEQ ID NO:1], of vector E425 for receiving heavy chain variable cDNA PCR products. The vector is digested with BsaI to generate the appropriate overhangs for ligation (CAGG; highlighted in bold), which is in-frame with the IgG CH1 sequence. The BsaI site downstream of the CMV promoter is highlighted in bold and generates the overhang to accept the ATGT of the cDNA PCR product.

FIG. 2 (A) shows a schematic diagram of ITS007-P31, which served as the template for PCR of GLP1R binding peptide-grafted antibodies using primers ET70 and ET71. Each of the peptide-grafted antibodies differed in the JH sequence used and in the intervening amino acids between the 3′ end of Exendin and the JH gene segment as a result of the initial V(D)J recombination which had resulted in deletions and/or additions at this junction; (B) shows the nucleic acid sequence [SEQ ID NO:2] of the PCR product that resulted from amplifying one peptide-grafted antibody in which 8 nucleotides (Ns) had been inserted between the 3′ Exendin sequences (underlined) and a JH5 gene segment (bold). The PCR product encompasses the histidine at the N-terminal end of Exendin through the SV40 poly(A) sequence.

FIG. 3 shows (A) the nucleic acid sequence [SEQ ID NO:50] and (B) the amino acid sequence [SEQ ID NO:51] for the agonist peptide-grafted HCAb ITS007-V125.

FIG. 4 shows (A) a schematic diagram and (B) a generic nucleic acid sequence [SEQ ID NO:3] of a fragment derived from P163 that contains a 5′ BamHI site, CMV promoter, a heavy chain variable segment (depicted by Ns), the 23 base pair RSS, spacer sequences and a 3′ BsmBI site. P163 represents a pool of 45 vectors each encoding a different VH gene segment upstream of the 23 bp RSS.

FIG. 5 shows the nucleic acid sequence [SEQ ID NO:4] of vector V610 digested with KpnI (bold) and BsaI (bold, overhang underlined).

FIG. 6 shows (A) a schematic diagram and (B) the nucleic acid sequence [SEQ ID NO:5] of the acceptor vector E501. E501 represents the final CDR optimization vector to diversify the junction of the 3′ VH framework and the 5′ Exendin peptide sequences. The BamHI and XhoI cloning site for P163 and ITS007-P31 are shown in bold.

FIG. 7 shows the nucleic acid sequence [SEQ ID NO:6] for the vector comprising the V503 expression cassette containing the coding sequence for the CRE recombinase (shown in bold).

FIG. 8 presents a flow chart showing steps in the method of generating Class B GPCR agonists in one embodiment of the invention.

FIG. 9 shows the results of FACS analysis of the HuTARG-L696 reporter cell line after culturing with and without Exendin (10 nM) for 24 hours. The cells were subsequently stained with 1 ug/ul biotinylated anti-GLP1R, 1 ug/ml SA647 and 1 ug/ml anti-CD19-PE conjugated antibody for one hour with rotation, washed one time with PBS containing 2% FBS and analyzed by flow cytometry using the C6 Accuri (BD).

FIG. 10 shows the results of FACS analysis of the recombined cell line HuTARG ITS007-L031, which surface expresses anti-GLP1R peptide-grafted antibodies, stained with anti-CD19-PE conjugated antibody and an anti-IgG Fc Alexa-647 conjugated antibody. Cells which were surface IgG positive and CD19 positive (exemplified by circle) were FACS sorted. Heavy chain sequences were PCR amplified from the FACS sorted cells and isolated clones were analyzed for agonist activity.

FIG. 11 shows the results of FACS analysis of HuTARG-L696 reporter cells that were transfected with expression vectors expressing an isolated exendin peptide-grafted antibody (ITS007-V107) or a negative control antibody, both with and without light chain. 48 hours post-transfection, surface kappa expression and CD19 reporter activation were determined by staining the samples with anti-Kappa Alexa488 and anti-CD19-PE conjugated antibodies.

FIG. 12 shows the results of FACS analysis of L696.19 GLP1r/cAMP reporter cells that were seeded 1:3 in a 24-well plate. The following day the cells were either left untreated (upper left panel), treated with 50 nM exendin (upper right panel), or incubated with culture supernatants, 1 μg/ml, of peptide-grafted heavy chain ITS007-V125 (lower left panel) or ITS007-V129 (histidine to alanine mutant; lower right panel). Two days later cells were stained with anti-CD19-PE and anti-human IgG-Fc-647 and analyzed by FACS on a C6 Accuri (BD).

FIG. 13 presents results showing the GLP1R agonist activity of the peptide-grafted heavy-chain ITS007-V125. ITS007-V125 was cloned into a secretion vector, expressed and purified. The reporter cell line was treated with different concentrations of ITS007-V125 followed by overnight incubation. (A) shows the dose dependent binding to the GLP-1 receptor (squares) and agonist activity (diamonds) expressed as the percentage of cells expressing the surface marker and determined by FACS analysis of reporter gene expression. (B) shows the specificity of ITS007-V125 for GLP1R. A commercial ELISA (cAMP Parameter Assay Kit (R&D Systems)) was used to measure cAMP concentration produced following incubation of a reporter cell line mock transfected or transfected with the GLP1 receptor. ITS007-V129 is a mutant that binds to, but does not activate, GLP1R.

FIG. 14 presents (A) the nucleotide sequence of ITS007-V105 (VH sequence through the end of the JH) [SEQ ID NO:23]; (B) the corresponding amino acid sequence of ITS007-V105 [SEQ ID NO:24]; (C) the nucleotide sequence of ITS007-V107 (VH sequence through the end of the JH) [SEQ ID NO:25], and (D) the corresponding amino acid sequence of ITS007-V107 [SEQ ID NO:26].

FIG. 15 presents a flow chart showing steps in the method of generating GLP1R agonists in one embodiment of the invention.

FIG. 16 presents an overview of the reporter cell line in accordance with an embodiment of the invention.

FIG. 17 presents the nucleotide sequence of vector V525 expressing the GLP1 receptor [SEQ ID NO:21].

FIG. 18 presents the nucleotide sequence of vector E518 comprising a cyclic AMP response element with minimal promoter and the CD19 reporter gene [SEQ ID NO:22].

FIG. 19 presents a sequence alignment of peptides of the vasoactive intestinal peptide (VIP)/secretin family. Sequence identity is represented by a black box, and sequence homologies are represented by grey boxes. Numbers indicate the length of the peptides. (from Couvineau and Laburthe, 2012, Br J Pharmacol., 166(1):42-50).

FIG. 20 presents the nucleic acid and amino acid sequences from VH to the end of JH for the variant HCAbs (A), (B) ITS007-V126 [SEQ ID NOs:53 & 54]; (C), (D), ITS007-V127 [SEQ ID NOs:55 & 56]; (E), (F) ITS007-V128 [SEQ ID NOs:57 & 58]; (G), (H) ITS007-V130 [SEQ ID NOs:59 & 60]; and (I), (J) ITS007-V212 [SEQ ID NOs:67 & 68].

FIG. 21 shows results of FACS analysis of reporter cells transfected with expression vectors expressing glucagon (ITS011-V36, -V66 and -V69: centre row) peptide-grafted antibodies, oxyntomodulin (ITS011-V23, -V100 and -V74: bottom row) peptide-grafted antibodies or control antibodies, pUC (Empty Vector), ITS011-V04 (Glc-Fc) or ITS011-V05 (OXY-Fc) (top row).

FIG. 22 presents a schematic diagram showing format options for (A) VH domain libraries, and (B) HCAb agonists in accordance with certain embodiments of the invention.

FIG. 23 shows (A) the results of FACS analysis of a HCAb variant library based on ITS007-V125, -V126 and -V130. The mutagenized library and original parent HCAbs prior to mutagenesis were stained with soluble N-terminal Biotinylated GLP1R for 1 hr and then washed and incubated with anti-Human IgG PE labelled antibody as a measure of cell surface HCAb expression and Streptavidin-Alexa 647 conjugate to assess degree of binding to soluble GLP1R, and (B) potency characterization of HCAbs identified from the library with higher affinity for GLP1R. Activity was measured in a cAMP reporter based assay described above and values listed are fold improvement over the original ITS007-V125 clone prior to mutagenesis.

FIG. 24 shows the nucleic acid and amino acid sequences from VH to the end of JH for the variant peptide-grafted HCAbs (A), (B) ITS007-V206 [SEQ ID NOs:61 & 62]; (C), (D) ITS007-V209 [SEQ ID NOs:63 & 64], and (E), (F) ITS007-V211 [SEQ ID NOs:65 & 66].

FIG. 25 shows the results of (A) the enrichment of reporter positive events for increased potency after three rounds of FACS sorting with (A) representing CDR3 mutants, and (B) representing CDR1 and CDR2 mutants, with increased activity.

FIG. 26 shows the improved GLP1R agonist activity of a HCAb in the VH² configuration in the 293-GLP1R reporter cell line.

FIG. 27 presents the nucleic acid sequence of the ITS007-V152 (tet) tetracycline inducible CMV promoter [SEQ ID NO:69]. Repressor binding sites are indicated in bold flanking the TATTA box.

FIG. 28 presents the nucleic acid and amino acid sequences from VH to the end of JH for the variant peptide-grafted HCAbs (A), (B) ITS007-V241 [SEQ ID NOs:70 & 71]; (C), (D) ITS007-V242 [SEQ ID NOs:72 & 73]; (E), (F) ITS007-V243 [SEQ ID NOs:74 & 75]; (G), (H) ITS007-V244 [SEQ ID NOs:76 & 77]; (I), (J) ITS007-V245 [SEQ ID NOs:78 & 79], and (K), (L) ITS007-V246 [SEQ ID NOs:80 & 81].

FIG. 29 presents the nucleic acid sequence for the VH² HCAb ITS007-V248 [SEQ ID NO:52].

FIG. 30 presents the nucleic acid and amino acid sequences from VH to the end of JH for the variant peptide-grafted HCAbs (A), (B) ITS011-V36 [SEQ ID NOs:97 & 98]; (C), (D) ITS011-V66 [SEQ ID NOs:99 & 100]; (E), (F) ITS011-V69 [SEQ ID NOs:101 & 102]; (G), (H) ITS011-V23 [SEQ ID NOs:103 & 104]; (I), (J) ITS011-V74 [SEQ ID NOs:105 & 106]; (K), (L) ITS011-V100 [SEQ ID NOs:107 & 108]; (M), (N) ITS011-V165 [SEQ ID NOs:109 & 110], and (O), (P) ITS011-V171 [SEQ ID NOs:111 & 112].

### DETAILED DESCRIPTION OF THE INVENTION

In co-owned International Patent Application No. PCT/CA2013/050204 (WO 2013/134881) it was demonstrated that ligands to GLP1R can be used to generate a peptide-grafted antibody that can bind to this receptor. Although these peptide-grafted antibodies were able to bind to the receptor, they were not able to activate the receptor. The methods described herein allow for the generation and identification of peptide-grafted antibody agonists to Class B G-protein coupled receptors (GPCRs), including the GLP1 receptor.

In addition to ensuring that the grafted peptide itself is in the correct orientation and conformation relative to the Ig scaffold, successful peptide grafting also requires that the structure of the IgG molecule itself is not adversely affected. Both pairing and expression are potentially compromised by the grafted peptide sequences. The methods described herein allow for grafting a peptide into the complete IgG scaffold. The methods further allow for interrogation of conformations within the context of the full human VH repertoire which also provides for additional conformational opportunities.

In certain embodiments, the methods described herein result in coupling of de novo protein engineering with a functional assay so that the variants can be assayed directly in the cells. While International Patent Application No. PCT/CA2013/050204 (WO 2013/134881) describes the ability to generate peptide-grafted variants within the full IgG scaffold so that the relevant sequence context and conformations generated represent the final drug candidate and does not require any additional reformatting, only binders were identified and the task of finding a rare agonist amongst these binders by known methods would not have been practical. The methods described herein allow for the screening of hundreds of millions of variants for a functional activity. In certain embodiments, these methods allow for functional screening of peptide-grafted variants within the full scaffold directly in a mammalian cell thus allowing for the isolation of rare variants with agonist activity. In certain embodiments, the methods allow for the generation of large numbers of variants differing in both length and composition of peptide flanking sequences directly within the complete Ig scaffold, and for assaying those variants for function without reformatting thus providing for the evaluation of an extremely large number of peptide-grafted variants and identification of peptide-grafted antibody agonists from amongst those variants.

In certain embodiments, the methods are based on an engineered mammalian cell line that uses V(D)J recombination to generate large libraries of peptide-grafted antibody variants, displays the variants on the cell surface for the purpose of antigen selection and indicates GPCR signalling by expressing a reporter gene that can be readily detected by standard methods, such as flow cytometry.

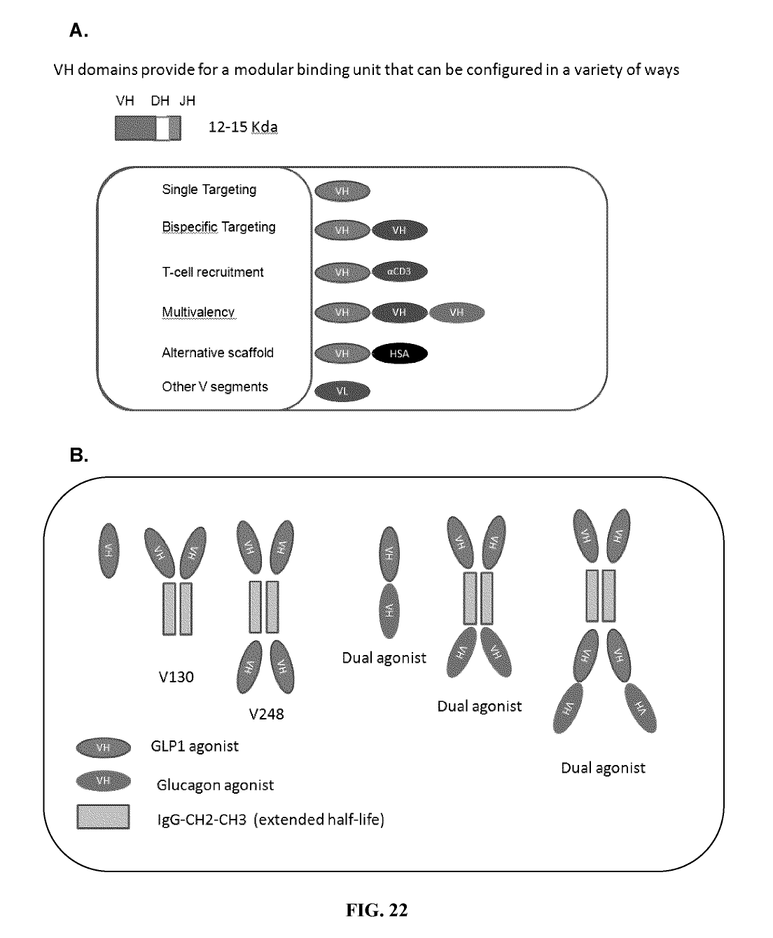

Certain embodiments of the invention relate to peptide-grafted antibody agonists of GPCRs identified by the methods disclosed herein. In some embodiments, the peptide-grafted antibody agonists are heavy-chain only antibodies. Some embodiments relate to isolated VH domains from these heavy-chain only antibodies and their use to prepare alternately formatted heavy-chain only antibodies, such as those shown in FIG. 22. Some embodiments relate to alternatively formatted heavy-chain only antibodies prepared from these isolated VH domains.

Some embodiments relates to peptide-grafted antibodies to GLP1R or the glucagon receptor, in which the grafted peptide has a sequence derived from one of the GLP1, exendin, glucagon or oxyntomodulin sequences. Amino terminal fusions to these peptides have been shown to negate activity of the peptide. As such, it was generally accepted in the field that a free amino terminus of the ligand was required for activation of the cognate receptor. As demonstrated herein, however, a free amino terminus is not required for agonist activity and using the methods disclosed herein, it is possible to generate a GLP1R or glucagon receptor agonist that contains additional amino acids at the amino terminus of the ligand. As demonstrated herein, a functional GLP1R or glucagon receptor agonist can be generated by peptide grafting of the ligand completely within an immunoglobulin.

### DEFINITIONS

Unless defined otherwise, all technical and scientific terms used herein have the same meaning as commonly understood by one of ordinary skill in the art to which this invention belongs.

As used herein, the term “about” refers to an approximately +/−10% variation from a given value. It is to be understood that such a variation is always included in any given value provided herein, whether or not it is specifically referred to.

The term “plurality” as used herein means more than one, for example, two or more, three or more, four or more, and the like.

“Naturally occurring,” as used herein, as applied to an object, refers to the fact that an object can be found in nature. For example, an organism, or a polypeptide or polynucleotide sequence that is present in an organism that can be isolated from a source in nature and which has not been intentionally modified by man in the laboratory is naturally occurring.

The term “isolated,” as used herein with reference to a material, means that the material is removed from its original environment (for example, the natural environment if it is naturally occurring). For example, a naturally occurring polynucleotide or polypeptide present in a living animal is not isolated, but the same polynucleotide or polypeptide separated from some or all of the co-existing materials in the natural system, is isolated. Such polynucleotides could be part of a vector and/or such polynucleotides or polypeptides could be part of a composition, and still be isolated in that such vector or composition is not part of its natural environment.

The term “recombination-competent” when used herein with reference to a host cell means that the host cell is capable of mediating RAG-1/RAG-2 recombination. The host cell may, therefore, express RAG-1 and RAG-2, or functional fragments thereof, or may be modified (for example, transformed or transfected with appropriate genetic constructs) such that it expresses RAG-1 and RAG-2, or functional fragments thereof. The expression of one or both of RAG-1 and RAG-2 in the recombination-competent host cell may be constitutive or it may be inducible. A recombination-competent host cell may optionally further express TdT, or a functional fragment thereof.

The use of the word “a” or “an” when used herein in conjunction with the term “comprising” may mean “one,” but it is also consistent with the meaning of “one or more,” “at least one” and “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

It is contemplated that any embodiment discussed herein can be implemented with respect to any method or composition of the invention, and vice versa. Furthermore, compositions and kits of the invention can be used to achieve methods of the invention.

### Peptide-Grafted Antibody Agonists

Certain embodiments of the invention relate to antibodies comprising one or more Class B GPCR agonist peptides grafted into one or more CDR regions. In certain embodiments these peptide-grafted antibody agonists are prepared by the methods disclosed herein.

Examples of Class B GPCRs that may be targeted include, but are not limited to, pituitary adenylate cyclase-activating polypeptide type 1 receptor (PACAPR), calcitonin receptor (CALCR), corticotropin-releasing hormone receptor (CRHR), glucose-dependent insulinotropic polypeptide receptor/gastric inhibitory polypeptide receptor (GIPR), glucagon receptor, glucagon-like peptide receptors (GLP1R and GLP2R), growth hormone releasing hormone receptor (GHRHR), parathyroid hormone receptor (PTHR), secretin receptor (SCTR), vasoactive intestinal peptide (VIP) receptor, brain-specific angiogenesis inhibitor (BAI), CD97 antigen (CD97), EMR hormone receptor, GPR56 orphan receptor, latrophilin receptor, diuretic hormone receptor and Ig-hepta receptor.

As is known in the art, Class B GPCRs can be further categorized into sub-groups (Harmar, (2001), Genome Biol., 2(12); reviews 3013.1-3013.10). Subfamily B1 comprises the classical hormone receptors including, for example, pituitary adenylate cyclase-activating polypeptide (PACAP) type 1 receptor, calcitonin receptor (CALCR), corticotropin-releasing hormone receptor (CRHR), glucose-dependent insulinotropic polypeptide receptor/gastric inhibitory polypeptide receptor (GIPR), glucagon receptor, glucagon-like peptide receptors (GLP1R and GLP2R), growth hormone releasing hormone receptor (GHRHR), parathyroid hormone receptor (PTHR), secretin receptor (SCTR) and vasoactive intestinal peptide (VIP) receptor. All members of the B1 subfamily are capable of regulating intracellular concentrations of cyclic AMP (cAMP). In certain embodiments, the peptide-grafted antibody agonists described herein comprise a peptide agonist of a Subfamily B1 GPCR.

Peptides included in the peptide-grafted antibodies may be derived from the sequence of a natural ligand for the target GPCR or from other known agonistic or binding peptides. For example, the peptide may include all or a fragment of the sequence of the natural ligand or known agonist or binder. In certain embodiments, the binding peptide may be identified by the methods described in International Patent Application No. PCT/CA2013/050204 (WO 2013/134881). In certain embodiments, it is also contemplated that antagonistic or inactive peptides may be used as a starting sequence with the methods described herein being used to introduce sufficient sequence diversity to result in a grafted peptide having agonist activity. Non-limiting examples of ligands to Subfamily B1 GPCRs are shown in Table 1A (see also, Harmar, 2001, ibid).

TABLE 1A
Ligands for Subfamily B1 Receptors

| Receptor | Ligands |
|---|---|
| Calcitonin receptor (CALCR) | Calcitonin |
| | Adrenomedullin |
| | Peptide histidine methionine |
| | (PHM) |
| Calcitonin receptor-like receptor | Calcitonin gene related peptide |
| (CALCRL) | Amylin |
| | CGRP |
| Corticotropin-releasing factor 1 | Corticotropin releasing factor |
| receptor | (CRF) |
| | Urocortin |
| Corticotropin-releasing factor 2 | Urocortin |
| receptor | Urocortin II |
| | Urocortin III |
| Glucose-dependent insulinotropic | Gastric inhibitory peptide (GIP) |
| polypeptide receptor/gastric | |
| inhibitory | |
| polypeptide receptor (GIPR) | |
| Glucagon receptor | Glucagon |
| | Oxyntomodulin |
| Glucagon-like peptide 1 receptor | GLP1 |
| (GLP1R) | Exendin |
| | Oxyntomodulin |
| Glucagon-like peptide 2 receptor | GLP-2 |
| (GLP2R) | |
| Growth hormone releasing | Growth hormone releasing |
| hormone receptor (GHRHR) | hormone |
| | Growth hormone releasing factor |
| | (GRF) |
| Parathyroid hormone 1 receptor | Parathyroid hormone (PTH) |
| (PTH1R) | PTH-related peptide |
| | PTH (1-34) peptide |
| Parathyroid hormone 2 receptor | TIP39 |
| (PTH2R) | |
| Secretin receptor (SCTR) | Secretin |
| Vasoactive intestinal peptide | Vasoactive intestinal peptide |
| receptor 1 (VPAC₁) | (VIP) |
| | Pituitary adenylate cyclase- |
| | activating polypeptide (PACAP) |
| | Helodermin |
| Vasoactive intestinal peptide | Vasoactive intestinal peptide |
| receptor 2 (VPAC₂) | (VIP) |
| | Pituitary adenylate cyclase- |
| | activating polypeptide (PACAP) |
| | Helodermin |
| Pituitary adenylate cyclase- | Pituitary adenylate cyclase- |
| activating polypeptide type I | activating polypeptide (PACAP) |
| receptor (PAC₁) | |

Glicentin is also a candidate ligand for the glucagon, GLP1 and/or GLP2 receptors.

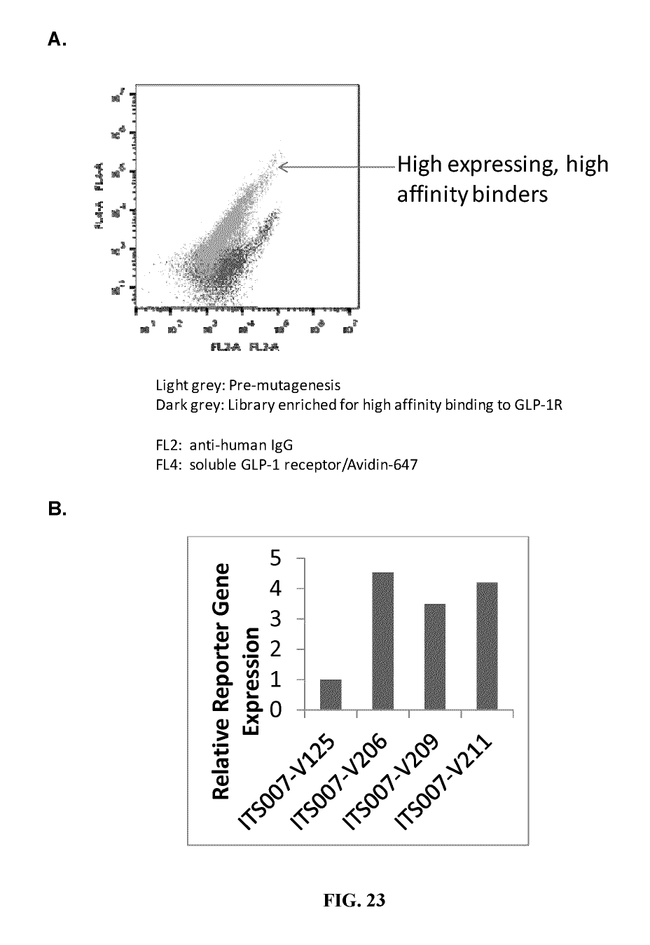

The amino acids sequences for various ligands and agonists of Class B GPCRs are known in the art and are available from publicly accessible databases such as the GenBank sequence database maintained by the National Center for Biotechnology Information. Exemplary sequences are provided in Table 1B below (see also FIG. 23):

TABLE 1B
Sequences of Ligands for Subfamily B1 Receptors

| | | SEQ |
|---|---|---|
| Ligand | Sequence | ID NO |
| Exendin-4 | HGEGRFTSDLSKQMEEEAVRLFI | 7 |
| | EWLKNGGPSSGAPPPS | |
| GLP1 | HAEGTFTSDVSSYLEGQAAKEFI | 8 |
| | AWLVKGR | |
| GLP2 | HADGSFSDEMNTILDNLAARDFI | 9 |
| | NWLIQTKITDR | |
| Glucagon | HSQGTFTSDYSKYLDSRRAQDRV | 10 |
| | QWLMNT | |
| Oxyntomodulin | HSQGTFTSDYSKYLDSRRAQDFV | 34 |
| | QWLMNTKRNRNNIA | |
| PACAP-38 | HSDGIFTDSYSRYRKQMAVKKYL | 11 |
| | AAVLGKRYKQRVKNK | |
| PTH | MIPAKDMAKVMIVMLAICFLTKS | 12 |
| | DGKSVKKRSVSEIQLMHNLGKHL | |
| | NSMERVEWLRKKLQDVHNFVALG | |
| | APLAPRDAGSQRPRKKEDNVLVE | |
| | SHEKSLGEADKADVNVLTKAKSQ | |
| VIP | HSDAVFTDNYTRLRKQMAVKKYL | 43 |
| | NSILN | |
| PACAP-27 | HSDGIFTDSYSRYRKQMAVKKYL | 44 |
| | AAVL | |
| Helodermin | HSDAIFTEEYSKLLAKLALQKYL | 45 |
| | ASILGSRTSPPP | |
| PHM | HADGVFTSDFSKLLGQLSAKKYL | 46 |
| | ESLM | |
| Secretin | HSDGTFTSELSRLREGARLQRLL | 47 |
| | QGLV | |
| GRF | YADAIFTNSYRKVLGQLSARKLL | 48 |
| | QDIMSRQQGESNQERGARARL | |
| GIP | YAEGTFISDYSIAMDKIHQQDFV | 49 |
| | NWLLAQKGKKNDWKHNITQ | |

Peptides corresponding to homologues of the ligands described above and in Table 1 are also known in the art.

The peptide-grafted antibody agonist may comprise a single peptide or it may comprise a plurality of peptides, for example, 2, 3, 4, 5 or 6 peptides. When the peptide-grafted antibody agonist comprises a plurality of peptides, the peptides may be grafted into the same CDR or into different CDRs. Preferably the peptides are grafted into different CDRs. The plurality of peptides may be agonists of the same GPCR or they may target different GPCRs. In certain embodiments, therefore, the peptide-grafted antibody agonist may have multiple specificities. For example, the peptide-grafted antibody agonist could be a dual agonist targeting two different GPCRs, such as GLP1R and the glucagon receptor.

In certain embodiments, the peptide-grafted antibody agonists described herein comprise a peptide agonist of a Subfamily B1 GPCR, for example, PACAP type 1 receptor, CALCR, CRHR, GIPR, glucagon receptor, GLP1R, GLP2R, GHRHR, PTHR, SCTR or VIP receptor. In some embodiments, the peptide-grafted antibody agonists comprise a peptide agonist of a Subfamily B1 GPCR and the peptide agonist comprises a sequence selected from any one of the peptide sequences shown in Table 1B, or a functional fragment thereof. In some embodiments, the peptide-grafted antibody agonists described herein comprise a peptide agonist of a Subfamily B1 GPCR, for example, PACAP type 1 receptor, CALCR, CRHR, glucagon receptor, GLP1R, GLP2R, SCTR or VIP receptor. In some embodiments, the peptide agonist comprises the sequence of exendin, GLP1, GLP2, glucagon, oxyntomodulin, PACAP-27, PACAP-38, VIP, helodermin, PHM or secretin, or a functional fragment thereof.

In certain embodiments of the invention, the peptide-grafted antibody agonists described herein comprise a peptide agonist of the GLP1 receptor. Agonist peptides suitable for targeting GLP1R include, for example, GLP1 [SEQ ID NO:8] and exendin [SEQ ID NO:7], as well as modified versions and functional fragments of these peptides.

In certain embodiments, the peptide-grafted antibody agonists described herein comprise a peptide agonist of the glucagon receptor. Agonist peptides suitable for targeting the glucagon receptor include, for example, glucagon [SEQ ID NO:10] and oxyntomodulin [SEQ ID NO:34], as well as modified versions and functional fragments of these peptides.

In some embodiments, the inserted peptide sequence includes an additional basic amino acid upstream and adjacent to the N-terminal amino acid of the peptide, for example an arginine or lysine. As shown herein, certain peptide sequences that comprise an N-terminal histidine benefit from the insertion of a basic amino acid immediately N-terminal to this histidine. In certain embodiments, therefore, when the native peptide sequence comprises an N-terminal histidine, the inserted peptide sequence includes an additional basic amino acid upstream and adjacent to the N-terminal amino acid, for example an arginine or lysine. Non-limiting examples of native peptide agonists of Class B GPCRs that comprise an N-terminal histidine are exendin, GLP1, GLP2, glucagon, oxyntomodulin, PACAP-27, PACAP-38, VIP, helodermin, PHM and secretin.

In certain embodiments, the inserted peptide sequence will include heterologous flanking sequences either at the N-terminal end, the C-terminal end, or at both. Typically, the flanking sequences will include between one and about 20 amino acids, for example, between one and about 18 amino acids, between one and about 15 amino acids, between one and about 12 amino acids, and between one and about 10 amino acids, or any amount therebetween.

In certain embodiments of the invention, the peptide-grafted antibody comprises a heterologous flanking sequence at the N-terminal end of the inserted peptide and optionally a heterologous flanking sequence at the C-terminal end. In some embodiments, the peptide-grafted antibody comprises a flanking sequence at the N-terminal end of the inserted peptide in which the amino acid proximal to the N-terminal amino acid of the peptide is a basic amino acid, and optionally further comprises a flanking sequence at the C-terminal end. In some embodiments, the peptide-grafted antibody comprises a flanking sequence at the N-terminal end of the inserted peptide in which the amino acid proximal to the N-terminal amino acid of the peptide is an arginine or lysine, and optionally further comprises a flanking sequence at the C-terminal end.

In certain embodiments, the peptide-grafted antibody optionally includes a heterologous flanking sequence at the C-terminal end, but does not include a heterologous flanking sequence at the N-terminal end of the inserted peptide. In some embodiments, the peptide-grafted antibody optionally comprises a flanking sequence at the C-terminal end, does not include a flanking sequence at the N-terminal end of the inserted peptide but is inserted within the CDR region such that the amino acid proximal to the N-terminal amino acid of the peptide is predefined. For example, the amino acid proximal to the N-terminal amino acid of the inserted peptide may be a basic amino acid. In some embodiments, the inserted peptide optionally comprises a flanking sequence at the C-terminal end, and is inserted into the CDR region such that the amino acid proximal to the N-terminal amino acid of the peptide is an arginine or lysine.

In certain embodiments, the peptide-grafted antibody comprises a peptide agonist of GLP1R or the glucagon receptor in which the N-terminal amino acid of the grafted peptide is a histidine. In some embodiments, the GLP1R or glucagon receptor peptide agonist comprises a heterologous flanking sequence at the N-terminal end of the inserted peptide and optionally a heterologous flanking sequence at the C-terminal end. In some embodiments, the GLP1R or glucagon receptor peptide agonist comprises a flanking sequence at the N-terminal end of the inserted peptide in which the amino acid proximal to the N-terminal amino acid of the peptide is a basic amino acid, and optionally further comprises a flanking sequence at the C-terminal end. In some embodiments, the GLP1R or glucagon receptor peptide agonist comprises a flanking sequence at the N-terminal end of the inserted peptide in which the amino acid proximal to the N-terminal amino acid of the peptide is an arginine or lysine, and optionally further comprises a flanking sequence at the C-terminal end. In some embodiments, the GLP1R or glucagon receptor peptide agonist optionally comprises a flanking sequence at the C-terminal end, does not include a flanking sequence at the N-terminal end of the inserted peptide but is inserted within the CDR region such that the amino acid proximal to the N-terminal amino acid of the peptide is a basic amino acid. In some embodiments, the GLP1R or glucagon receptor peptide agonist optionally comprises a flanking sequence at the C-terminal end, and is inserted into the CDR region such that the amino acid proximal to the N-terminal amino acid of the peptide is an arginine or lysine.

A peptide that is described as being inserted into a CDR region may be inserted within the CDR such that the amino acids that make up the CDR are retained, or it may replace part or all of the CDR. The peptide-grafted antibodies may comprise an agonist peptide inserted into, or replacing part or all of, one CDR, or it may comprise a plurality of peptides with each peptide inserted into, or replacing part or all of, a CDR. The peptide-grafted antibodies may comprise a peptide grafted into a heavy chain CDR or into a light chain CDR or into both. In some embodiments, the peptide-grafted antibodies may comprise a peptide grafted into a heavy chain CDR3 or into a light chain CDR3 or into both. In certain embodiments, the peptide-grafted antibodies may comprise a peptide grafted into at least a heavy chain CDR. In some embodiments, the peptide-grafted antibodies may comprise a peptide grafted into the heavy chain CDR3. In some embodiments, the peptide replaces the D segment in the heavy chain CDR3.

The final peptide-grafted antibody may be a full-length immunoglobulin (such as a full-length IgA, IgA2, IgD, IgE, IgG (i.e. an IgG1, IgG2, IgG3 or IgG4) or IgM) or an immunoglobulin fragment (such as a Fab, Fab′, F(ab′)₂, Fd, Fvor single-chain Fv (scFv) fragment, or a single domain antibody). Full-length immunoglobulins may comprise both a heavy chain and a light chain, or they may include just a heavy chain or just a light chain. Certain embodiments of the invention relate to peptide-grafted antibodies which are full-length IgG immunoglobulins. In some embodiments, the peptide-grafted antibody comprises just the heavy chain of a full-length IgG immunoglobulin, or a functional fragment thereof.

In some embodiments, the peptide-grafted antibody is a heavy chain only antibody (HCAb). In this context, a HCAb may include the VH domain together with the CH1, CH2 and CH3 regions, or it may contain the VH domain and the CH2 and CH3 regions only. In the latter conformation, the VH domain is attached directly to the hinge region of the antibody. The HCAb may include two “arms” of the antibody (i.e. one arm comprising a VH domain and constant regions linked via a disulphide bridge to a second arm comprising a VH domain and constant regions), or it may include just a single arm (i.e. one arm comprising a VH domain and constant regions). Fragments of HCAbs are also contemplated, for example, fragments including two VH domains and the hinge region only without any constant regions, or a single VH domain.

The modular nature of the fully human VH single domain makes it a versatile building block for generating alternative format HCAbs that may find use as therapeutics. The VH single domain is approximately 12-15 KDa and contains only the variable gene segment through to the end of the JH gene segment. Certain embodiments relate to alternative format HCAbs comprising two or more VH domains as outlined in FIG. 22A & B. The alternative format HCAbs may be monospecific or they may be bispecific or they may be multispecific. For example, in some embodiments, the alternative format HCAb may comprise two or more linked VH domains, each domain comprising a different peptide agonist, and each peptide agonist targeted to a different GPCR. In some embodiments, these VH domains may be attached to constant regions. In some embodiments, the alternative format HCAbs may comprise a CH2 region, a CH3 region, a first VH domain attached to the CH2 region and one or more VH domains attached to the CH3 region. In this latter format the VH domains may be identical or different. When the VH domains are different they may, for example, target different GPCR thus providing an HCAb with dual or multi-specificity. In some embodiments, one or more VH domains may be attached to an alternate scaffold, such as human serum albumin (HSA).

In certain embodiments, the peptide-grafted antibody is an HCAb comprising one or more Class B GPCR agonist peptides grafted into one or more CDR regions. In some embodiments, the peptide-grafted antibody is an HCAb comprising a plurality of Class B GPCR agonist peptides grafted into one or more CDR regions. In some embodiments, the peptide-grafted antibody is an HCAb comprising a plurality of Class B GPCR agonist peptides each peptide grafted into a different CDR region. In some embodiments, the peptide-grafted antibody is an HCAb comprising a peptide targeting GLPR and a peptide targeting the glucagon receptor.

Although the foregoing description has focussed primarily on the use of V(D)J recombination to peptide graft into the heavy chain variable region, it will be readily appreciated that the same approach can be used with the light chain variable region simply by replacing the heavy chain VH and JH gene segments with the light chain variable (VL) and joining (JL) gene segments. In such embodiments, the peptide sequence may be used as an artificial D gene segment. Certain embodiments, therefore, relate to peptide-grafted agonist antibodies comprising one or more peptides grafted into the light chain variable region. These peptide-grafted antibodies may optionally further comprise one or more peptides grafted into their cognate heavy chain variable regions.

The peptide-grafted agonist antibodies and fragments thereof have utility in a variety of contexts including as therapeutics, diagnostics and as research reagents. For example, peptide-grafted agonists of GLP1R, GLP2R or the glucagon receptor may find therapeutic use in the management of diabetes and/or weight loss; peptide-grafted agonists of PTH1R may find therapeutic application in the treatment of osteoporosis; and peptide-grafted agonists of VPAC₁ or VPAC₂ have potential as therapeutics for inflammatory and neurodegenerative diseases. In addition, the antibodies or fragments thereof may find use in diagnostic applications, for example when labelled, the antibodies or fragments may be used as diagnostic and/or imaging agents.

### Methods

Certain embodiments of the invention relate to high-throughput methods of identifying peptide-grafted antibody agonists of Class B GPCRs. The methods make use of a reporter cell line that expresses the target GPCR and also includes coding sequences for a surface-expressed reporter protein under control of a cAMP responsive promoter. Typically, the reporter cell line may be a mammalian cell line. In certain embodiments, the cell line is a human cell line.

In general, the methods described herein start with a library of polynucleotides encoding peptide-grafted antibodies, each antibody comprising at least one GPCR-targeting peptide grafted into a CDR region. Preferably at least some members of the library will have been shown to bind to the target GPCR. The library may have been generated by various art known methods, including methods incorporating random mutagenesis and/or phage display. In certain embodiments, the library is generated using the de novo antibody generation methods described in International Patent Application No. PCT/CA2013/050204 (WO 2013/134881). This approach is based on generation of V(D)J recombination substrates which comprise the full repertoire of human VH and JH sequences or the full repertoire of VL and JL sequences and in which the peptide sequences, with optional flanking sequences, replace the D segments or in the case of VL and JL sequences, function as artificial D segments. The substrates are introduced into recombination competent host cells and allowed to undergo recombination. The approach thus allows introduction of diversity into the regions flanking the inserted peptide through V(D)J recombination with simultaneous interrogation of conformations of the grafted peptide within the context of the full human VH or VL repertoire.

In general when the peptide-grafted antibodies are heavy chain only antibodies, the library of peptide-grafted antibodies may be generated using the full repertoire of VH sequences. However, the use of a restricted number of VH gene segments is contemplated in some embodiments. Examples of restricted sets would include sets comprising one or a combination of the following VH gene segments that have been shown to express well in the HCAb format: VH1-8, VH3-11, VH3-15, VH3-20, VH3-30 and VH4-34.

The initial library of polynucleotides encoding peptide-grafted antibodies is screened functionally using the reporter cell line to identify those peptide-grafted antibodies having agonist activity against the target GPCR. The library may be screened directly, or the peptide-grafted antibody encoding polynucleotides may be subjected to a further V(D)J recombination step to increase the diversity of the library.

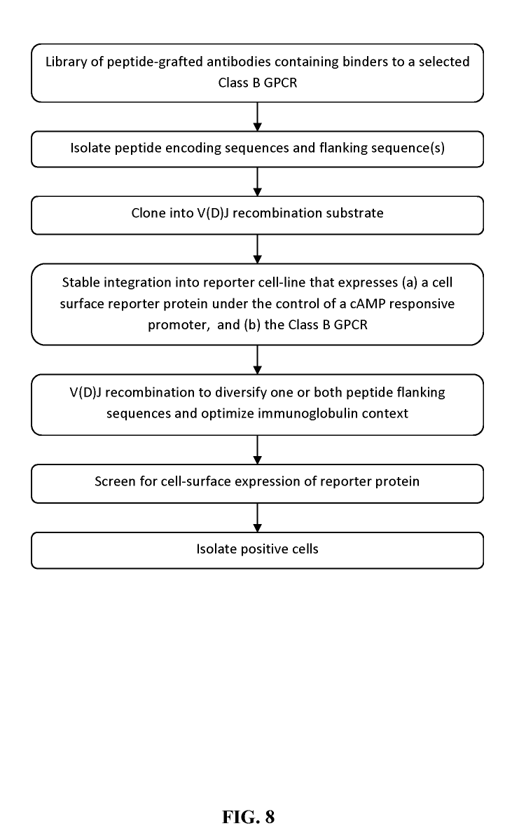

In certain embodiments, the peptide-grafted antibodies are subjected to a further V(D)J recombination step. An overview of the steps in an exemplary method comprising a further V(D)J recombination step is provided in FIG. 8. The V(D)J recombination step may introduce diversity at both ends of the grafted peptide, or it may be targeted to introduce diversity at just one terminus of the grafted peptide. As the N-terminal region of certain GPCR agonists is believed to be important for activity, in some embodiments, it may be advantageous to target the additional V(D)J recombination step to introduce additional diversity at the N-terminal end of the grafted peptide.

The V(D)J recombination step is carried out essentially as described in International Patent Application No. PCT/CA2013/050204 (WO 2013/134881). For this step, sequences encoding the grafted peptide and flanking regions as appropriate are amplified from the library with primers that introduce the required RSS sequence or sequences for RAG-mediated recombination, and the amplified sequences are subsequently ligated into a V(D)J substrate polynucleotide that comprises sequences encoding one or a plurality of VH segments and/or one or a plurality of JH segments, together with the required RSS sequences, or sequences encoding one or a plurality of VL segments and/or one or a plurality of JL segments, together with the required RSS sequences. One skilled in the art will appreciate that if additional sequence diversity is to be introduced to one end of the grafted peptide, then the amplified sequences from the original polynucleotide encoding the peptide-grafted antibody will include sequences adjacent to the terminus that is not being diversified. For example, if additional diversity is to be targeted into the N-terminal region of the grafted peptide, then the amplified sequences from the original polynucleotide encoding the peptide-grafted antibody may include the peptide-JH or peptide-JL junction and JH or JL sequences, and the V(D)J substrate polynucleotide may include sequences encoding one or a plurality of VH or VL segments only. Likewise, if additional diversity is to be targeted to the C-terminal region of the grafted peptide, then the amplified sequences from the original polynucleotide encoding the peptide-grafted antibody may include the VH-peptide or VL-peptide junction and VH or VL sequences, and the V(D)J substrate polynucleotide may include sequences encoding one or a plurality of JH or JL segments only.

The primers used to amplify the sequences from the original polynucleotide encoding the peptide-grafted antibody may optionally comprise degenerate sequences in order to introduce additional diversity into the sequences flanking the peptide. Various degenerate sequences suitable for introducing diversity are known in the art. Examples of degenerate sequences that may be used in certain embodiments include, but are not limited to, NNK, NNN, NNR, NNY and NNS, wherein N is any nucleotide, K is guanine or thymine, R is guanine or adenine, Y is thymine or cytosine, and S is guanine or cytosine.

Suitable RSS sequences for incorporation into the primers and V(D)J substrate polynuceotide are known in the art. The RSS sequences preferably consist of two conserved sequences (for example, heptamer, 5′-CACAGTG-3′, and nonamer, 5′-ACAAAAACC-3′), separated by a spacer of either 12+/−1 bp (a “12-signal” RSS) or 23+/−1 bp (a “23-signal” RSS). Other functional RSS sequences are known in the art. Examples of such RSS sequences, including their characterization as high, medium or low efficiency RSSs, are presented in Table 2 and 3.

TABLE 2
Exemplary Recombination Signal Sequences
(12 nucleotide spacer)

| | | |
|---|---|---|
| Heptamer | Spacer | Nonamer |
| H12 | S12 | N12 |

Part I. Efficiency: HIGH

| | | | |
|---|---|---|---|
| 1 | CACAGTG | ATACAGACCTTA | ACAAAAACC |
| | | [SEQ ID NO: 13] | |
| 2 | CACAGTG | CTACAGACTGGA | ACAAAAACC |
| | | [SEQ ID NO: 14] | |
| 3 | CACAGTG | CTCCAGGGCTGA | ACAAAAACC |
| | | [SEQ ID NO: 15] | |
| 4 | CACAGTG | CTACAGACTGGA | ACAAAAACC |
| | | [SEQ ID NO: 14] | |
| 5 | CACAGTG | CTACAGACTGGA | ACAAAAACC |
| | | [SEQ ID NO: 14] | |
| 6 | CACAGTG | CTACAGACTGGA | ACAAAAACC |
| | | [SEQ ID NO: 14] | |
| 7 | CACAGTG | GTACAGACCAAT | ACAGAAACC |
| | | [SEQ ID NO: 16] | |

Part II Efficiency: MEDIUM (~10-20% of High)

| | | | |
|---|---|---|---|
| 8 | CACGGTG | CTACAGACTGGA | ACAAAAACC |
| | | [SEQ ID NO: 14] | |
| 9 | CACAATG | CTACAGACTGGA | ACAAAAACC |
| | | [SEQ ID NO: 14] | |
| 10 | CACAGCG | CTACAGACTGGA | ACAAAAACC |
| | | [SEQ ID NO: 14] | |
| 11 | CACAGTG | CTACAGACTGGA | ACAAAAACC |
| | | [SEQ ID NO: 14] | |
| 12 | CACAGTG | CTACAGACTGGA | ACAAAAACC |
| | | [SEQ ID NO: 14] | |
| 13 | CACAGTG | CTACAGACTGGA | ACAAAAACC |
| | | [SEQ ID NO: 14] | |
| 14 | CACAGTG | CTACAGACTGGA | ACAAAAACC |
| | | [SEQ ID NO: 14] | |
| 15 | CACAGTG | CTACAGACTGGA | ACAAAAACC |
| | | [SEQ ID NO: 14] | |
| 16 | CACAGTG | CTACAGACTGGA | ACAAAAACC |
| | | [SEQ ID NO: 14] | |
| 17 | CACAGTG | CTACAGACTGGA | CAAAAACCC |
| | | [SEQ ID NO: 14] | |
| 18 | CACAGTG | CTACAGACTGGA | ACAAAAACC |
| | | [SEQ ID NO: 14] | |
| 19 | CACAATG | CTACAGACTGGA | ACAAAAACC |
| | | [SEQ ID NO: 14] | |
| 20 | CACAGCG | CTACAGACTGGA | ACAAAAACC |
| | | [SEQ ID NO: 14] | |

Part III. Efficiency: LOW (~1% or less of High)

| | | | |
|---|---|---|---|
| 21 | TACAGTG | CTACAGACTGGA | ACAAAAACC |
| | | [SEQ ID NO: 14] | |
| 22 | GACAGTG | CTACAGACTGGA | ACAAAAACC |
| | | [SEQ ID NO: 14] | |
| 23 | CATAGTG | CTACAGACTGGA | ACAAAAACC |
| | | [SEQ ID NO: 14] | |
| 24 | CACAATG | CTACAGACTGGA | ACAAAAACC |
| | | [SEQ ID NO: 14] | |
| 25 | CACAGTG | CTACAGACTGGA | ACAAAAACC |
| | | [SEQ ID NO: 14] | |
| 26 | CAGAGTG | CTCCAGGGCTGA | ACAAAAACC |
| | | [SEQ ID NO: 15] | |
| 27 | CACAGTG | CTCCAGGGCTGA | AAAAAAACC |
| | | [SEQ ID NO: 15] | |
| 28 | CTCAGTG | CTCCAGGGCTGA | ACAAAAACC |
| | | [SEQ ID NO: 15] | |

TABLE 3
Exemplary Recombination Signal Sequences
(23 Nucleotide Spacer)

| | | | |
|---|---|---|---|
| Heptamer | Spacer | Nonamer | |
| H23 | S23 | N23 | Ref.* |

Part I. Efficiency: HIGH

| | | | | |
|---|---|---|---|---|
| 1 | CACAGTG | GTAGTACTCCAC | ACAAAAACC | 4 |
| | | TGTCTGGCTGT | | |
| | | [SEQ ID NO: 17] | | |
| 2 | CACAGTG | GTAGTACTCCAC | ACAAAAACC | 3 |
| | | TGTCTGGCTGT | | |
| | | [SEQ ID NO: 17] | | |
| 3 | CACAGTG | GTAGTACTCCAC | ACAAAAACC | 1 |
| | | TGTCTGGGTGT | | |
| | | [SEQ ID NO: 17] | | |
| 4 | CACAGTG | TTGCAACCACAT | ACAAAAACC | 2 |
| | | CCTGAGTGTGT | | |
| | | [SEQ ID NO: 18] | | |
| 5 | CACAGTG | GTAGTACTCCAC | ACAAAAACC | 2 |
| | | TGTCTGGCTGT | | |
| | | [SEQ ID NO: 17] | | |
| 6 | CACAGTG | ACGGAGATAAAG | ACAAAAACC | 2 |
| | | GAGGAAGCAGG | | |
| | | [SEQ ID NO: 19] | | |
| 7 | CACAGTG | GCCGGGCCCCGC | ACAAAAACC | 5 |
| | | GGCCCGGCGGC | | |
| | | [SEQ ID NO: 20] | | |

Part II. Efficiency: MEDIUM (~10-20% of High)

| | | | | |
|---|---|---|---|---|
| 8 | CACAGTG | GTAGTACTCCAC | ACAAAAACC | 3 |
| | | TGTCTGGCTGT | | |
| | | [SEQ ID NO: 17] | | |
| 9 | CACAGTG | GTAGTACTCCAC | ACAAAAACC | 3 |
| | | TGTCTGGCTGT | | |
| | | [SEQ ID NO: 17] | | |
| 10 | CACAGTG | GTAGTACTCCAC | ACAAAAACC | 3 |
| | | TGTCTGGCTGT | | |
| | | [SEQ ID NO: 17] | | |
| 11 | CACAATG | GTAGTACTCCAC | ACAAAAACC | 3 |
| | | TGTCTGGCTGT | | |
| | | [SEQ ID NO: 17] | | |
| 12 | CACAGCG | GTAGTACTCCAC | ACAAAAACC | 3 |
| | | TGTCTGGCTGT | | |
| | | [SEQ ID NO: 17] | | |
| 13 | CACAGTA | GTAGTACTCCAC | ACAAAAACC | 3 |
| | | TGTCTGGCTGT | | |
| | | [SEQ ID NO: 17] | | |
| 14 | CACAGTG | GTAGTACTCCAC | ACAATAACC | 3 |
| | | TGTCTGGCTGT | | |
| | | [SEQ ID NO: 17] | | |
| 15 | CACAGTG | GTAGTACTCCAC | ACAAGAACC | 3 |
| | | TGTCTGGCTGT | | |
| | | [SEQ ID NO: 17] | | |
| 16 | CACAGTG | GTAGTACTCCAC | ACACGAACC | 3 |
| | | TGTCTGGCTGT | | |
| | | [SEQ ID NO: 17] | | |
| 17 | CACAGTG | GTAGTACTCCAC | ACAAAAACC | 3 |
| | | TGTCTGGCTGT | | |
| | | [SEQ ID NO: 17] | | |
| 18 | CACAGTG | GTAGTACTCCAC | ACACGAACC | 3 |
| | | TGTCTGGCTGT | | |
| | | [SEQ ID NO: 17] | | |
| 19 | CACAATG | GTAGTACTCCAC | ACAAAAACC | 3 |
| | | TGTCTGGCTGT | | |
| | | [SEQ ID NO: 17] | | |
| 20 | CACAGCG | GTAGTACTCCAC | ACAAAAACC | 3 |
| | | TGTCTGGCTGT | | |
| | | [SEQ ID NO: 17] | | |

Part III. Efficiency: LOW (~1% or less of High)

| | | | | |
|---|---|---|---|---|
| 21 | CACAGTA | GTAGTACTCCAC | ACAAAAACC | 3 |
| | | TGTCTGGCTGT | | |
| | | [SEQ ID NO: 17] | | |
| 22 | CACAGTG | GTAGTACTCCAC | ACAAAAACC | 3 |
| | | TGTCTGGCTGT | | |
| | | [SEQ ID NO: 17] | | |
| 23 | CACAATG | GTAGTACTCCAC | ACAAAAACC | 3 |
| | | TGTCTGGCTGT | | |
| | | [SEQ ID NO: 17] | | |
| 24 | CATAGTG | GTAGTACTCCAC | ACAAAAACC | 3 |
| | | TGTCTGGCTGT | | |
| | | [SEQ ID NO: 17] | | |
| 25 | CACAGTG | GTAGTACTCCAC | TGTCTCTGA | 3 |
| | | TGTCTGGCTGT | | |
| | | [SEQ ID NO: 17] | | |
| 26 | CACAGTG | GTAGTACTCCAC | ACAAAAACC | 1 |
| | | TGTCTGGGTGT | | |
| | | [SEQ ID NO: 17] | | |
| 27 | CACAGTG | GTAGTACTCCAC | ACAAAAACC | 1 |
| | | TGTCTGGGTGT | | |
| | | [SEQ ID NO: 17] | | |
| 28 | CACAGTG | GTAGTACTCCAC | ACAAAAACC | 1 |
| | | TGTCTGGGTGT | | |
| | | [SEQ ID NO: 17] | | |

*(1) Akamatsu, 1994, ibid; (2) Cowell, 2004, ibid; (3) Hesse, 1989 ibid; (4) Lee, 2003, ibid; (5) Nadel, 1998, ibid.

The V(D)J substrate polynucleotide into which the amplified sequences are ligated may further comprise one or more regulatory sequences allowing for expression of the peptide-grafted antibody once recombination has taken place, such as promoters, enhancers, terminators, alpha-factors, ribosome binding sites, polyadenylation signals and the like. Preferably, the V(D)J substrate polynucleotide further comprises additional coding sequences that encode a plasma membrane anchor domain such that the expressed peptide-grafted antibody is expressed on the surface of the reporter cell line, although secreted peptide-grafted antibodies are contemplated in some embodiments. The V(D)J substrate polynucleotide may already be part of an appropriate vector for transfection into the reporter cell line, or the V(D)J substrate polynucleotide comprising the amplified sequences may subsequently be cloned into an appropriate vector for transfection into the reporter cell line.

When the library of peptide-grafted antibodies is not subjected to an additional V(D)J recombination step, the coding sequence for the peptide-grafted antibody may in some embodiments be ligated to additional coding sequences that encode a plasma membrane anchor domain such that the peptide-grafted antibody is expressed on the surface of the host cell, for example, by cloning the coding sequence for the peptide-grafted antibody into a vector that provides the additional plasma membrane anchor domain coding sequences.

The vector used to transfect the reporter cell line may optionally include coding sequences for the antibody light chain under appropriate expression control sequences, such that an intact antibody is expressed from the vector in the reporter cell line. Alternatively, the reporter cell line may subsequently be transfected with a vector comprising coding sequences for the light chain under appropriate expression control sequences.

The reporter cell line used in the methods described herein comprises a first stably integrated recombinant nucleic acid construct comprising a reporter gene encoding a cell surface expressed protein that is under the control of a cAMP responsive promoter such that expression of the protein takes place only in the presence of cAMP. A cAMP responsive promoter is one that includes at least one cAMP response element (CRE) and, optionally, other upstream activators. A variety of cAMP responsive promoters are known in the art. For example, cAMP response element binding protein (CREB) has been shown to occupy over 4000 sites in the human genome (Zhang et al., 2005, PNAS, 102(12):4459-4464). The cAMP responsive promoter may be a naturally occurring cAMP responsive promoter, or it may be a promoter sequence genetically engineered to include at least one CRE and, where necessary, other upstream activators. The CRE may be a full-length CRE (for example: TGACGTCA) or it may be a partial CRE (for example, TGACG or CGTCA).

The cell line also includes a second stably integrated recombinant nucleic acid construct that comprises a sequence encoding the target Class B GPCR under control of an appropriate promoter. The promoter may be a constitutive promoter or it may be an inducible promoter. In certain embodiments, a recombinant nucleic acid construct that comprises a sequence encoding the target Class B GPCR under control of a constitutive promoter is used. The cell line is transfected with the vectors comprising the sequences encoding the peptide-grafted antibodies, cultured under conditions permitting expression of the peptide-grafted antibodies and expression of the Class B GPCR, and subsequently screened for surface expression of the protein encoded by the reporter gene. Cells expressing reporter gene comprise peptide-grafted antibodies having GPCR agonist activity. These cells can subsequently be isolated and the agonist peptide-grafted antibodies can optionally be further characterized, for example by sequencing.

The sequences encoding the peptide-grafted antibody agonist may be isolated for further downstream uses including, for example, sub-cloning into alternate expression vectors, further testing prior to therapeutic use and/or large scale production of the peptide-grafted antibody agonist.

The cell surface expressed protein encoded by the reporter gene included in the cell line may be one of a number of known surface expressed proteins or peptides for which an adequate detection method is available or can be readily designed. Examples include, but are not limited to, CD4, CD8, CD3, CD19, V5, FLAG or any epitope that is expressed on the surface of the cell and can be detected by a reagent. In certain embodiments of the invention, the cell surface expressed protein encoded by the reporter gene is CD19.

Various detection methods appropriate for high-throughput assays are known in the art. Typically, these are flow cytometry based and involve the use of antibodies or ligands conjugated to detectable labels. In certain embodiments, it may be desirable to use detectable labels that also permit enrichment of positive cells, for example, via magnetic bead enrichment.

In certain embodiments, the reporter cell line is also recombination competent such that the additional V(D)J recombination step described above for introducing additional diversity into one or both of the peptide flanking sequences may be conducted in the same cell line as the functional assay. In this context, the reporter cell line is capable of mediating RAG-1/RAG-2 recombination either via stably integrated nucleic acid constructs that express RAG-1 and RAG-2, or functional fragments thereof, or via transfection of the cell line with appropriate genetic constructs encoding RAG-1 and RAG-2, or functional fragments thereof. The expression of one or both of RAG-1 and RAG-2 in the recombination competent reporter cell line may be constitutive or it may be inducible. The recombination competent reporter cell line may optionally further express TdT, or a functional fragment thereof.

In certain embodiments relating to the use of a recombination competent reporter cell line, the reporter cell line may further comprise sites engineered into the chromosome that permit stable chromosomal integration of the V(D)J recombination substrate polynucleotide, for example, CRE/Lox recombination sites, FLP/FRT recombination sites, attP/attB recombination sites or combinations thereof. Approaches such as froxing and floxing have also been reported and may be appropriate in some embodiments. Accordingly, in some embodiments, the method may comprise amplifying sequences encoding the grafted peptide and one or both flanking regions from a library of peptide-grafted antibodies capable of binding the target Class B GPCR with primers that introduce the required RSS sequence or sequences for RAG-mediated recombination. The primers may optionally include degenerate sequences for further diversification. The amplified sequences are subsequently ligated into a V(D)J substrate polynucleotide that comprises sequences encoding one or a plurality of VH segments and/or one or a plurality of JH segments, or sequences encoding one or a plurality of VL segments and/or one or a plurality of JL segments, together with the required RSS sequences. The V(D)J substrate polynucleotide preferably further comprises additional coding sequences that encode a plasma anchor domain. The V(D)J substrate polynucleotide comprising the diversified peptide sequence is transfected into the reporter cell line under conditions that permit integration of the substrate into the corresponding site(s) engineered into the chromosome. The reporter cell is cultured under conditions that allow RAG-mediated recombination and expression of the resulting peptide-grafted antibodies. If a plasma membrane anchor domain was used, the antibodies are expressed on the cell surface. The cells can then be screened for expression of the reporter gene.

In certain embodiment, once a peptide-grafted antibody agonist has been identified using the methods described above, the method may further comprise generating improved peptide-grafted antibody agonists from the initial antibody, for example, by using the V(D)J protein optimization techniques as described in International Patent Application PCT/CA2013/050203 (WO 2013/134880). For example, V(D)J protein optimization may be used to target specific amino acids in the CDR into which the peptide is grafted such that a library of polynucleotides encoding variant peptide-grafted antibodies is generated that differ in the sequences of this CDR. The library of polynucleotides encoding variant peptide-grafted antibodies may then be screened functionally using the reporter cell line to identify those peptide-grafted antibodies having increased agonist activity against the target GPCR.

### Kits

Certain embodiments of the invention relate to kits comprising the reporter cell line disclosed herein for use in high-throughput methods of identifying agonists of Class B GPCRs, including peptide-grafted antibody agonists. The kits may optionally comprise reagents for detecting the reporter protein and, for peptide-grafted antibody agonists, may further optionally comprise a V(D)J substrate polynucleotide, as described above.

Optionally, for peptide-grafted antibody agonists, the kit may additionally comprise vectors encoding one or more of RAG-1, RAG-2 and TdT that are suitable for transfecting the reporter cell line such that the cell line expresses, or is capable of expressing, RAG-1, RAG-2 and/or TdT. Alternatively, when the cell line is recombination competent, the kit may optionally comprise a vector encoding TdT.

The kit may further comprise one or more additional components to assist with cloning and/or transfection and/or reporter detection, such as buffers, enzymes, selection reagents, growth media and the like.

One or more of the components of the kit may optionally be lyophilised and the kit may further comprise reagents suitable for the reconstitution of the lyophilised components. Individual components of the kit would be packaged in separate containers and, associated with such containers, can be instructions for use. The instructions for use may be provided in paper form or in computer-readable form, such as a CD, DVD or the like.

To gain a better understanding of the invention described herein, the following examples are set forth. It will be understood that these examples are intended to describe illustrative embodiments of the invention and are not intended to limit the scope of the invention in any way.

### EXAMPLES

Utilizing in vitro V(D)J mediated peptide-grafting methods as described in International Patent Application No. PCT/CA2013/050204 (WO 2013/134881), several hundred GLP1 receptor binders were generated. Forty-eight of these peptide-grafted antibodies were tested for agonist activity, but none was detected. Although conformational contexts were identified by these methods that were appropriate for binding to the receptor, the conformations were not appropriate for activating the receptor. As GLP1 peptide-Fc fusion proteins are active and a free amino terminus has been hypothesized by others to be essential for activity of the peptide, an approach of generating additional diversity at the amino terminal antibody-peptide junction in the isolated peptide-grafted antibodies that bound to the GLP1 receptor was adopted in an effort to find a conformational solution that would not require a free amino terminus. Briefly, the pool of GLP1 receptor binders was cloned into a second V(D)J diversification substrate to specifically target additional diversity to the amino terminal junction of the antibody scaffold and grafted peptide. V(D)J optimization vectors as described in International Patent Application No. PCT/CA2013/050203 (WO 2013/134880) were employed to target mutations and a library of approximately 100 million variants was generated.

To screen this many variants using traditional methods would not have been practical. In addition to creating an agonist peptide in a final antibody scaffold format and thus avoiding the requirement to reformat the antibody from phage format, generating diversity de novo in a mammalian cell also allows the mammalian cell line to be engineered to incorporate a functional reporter for cAMP activation. The ability to incorporate a cAMP functional reporter directly into the mammalian cell line capable of de novo diversity generation was an important factor in developing the assay described below that allowed a rare agonist to be identified from amongst a large pool of a receptor binders.

Although a number of different cAMP reporter assays have been previously described, those using reporter gene expression generally utilize luciferase. Luciferase-based reporter systems are enzymatic and give very high sensitivity. These systems often use destabilized enzyme to improve signal to noise ratios. In order to identify rare binders in the context described below, however, a reporter system that resulted in expression of the reporter on the cell surface was preferred. This would allow, for example, for magnetic bead enrichments. Whether or not a cell surface reporter system linked to cAMP levels would be possible was not clear prior to developing the assay described below. For example, it was not known whether expression driven from a minimal promoter containing CREs would be sufficient to generate levels useful for FACS or magnetic sorting. It was also unknown if constitutive reporter gene expression would result in down-regulation of the receptor over time.

In addition, the peptide-grafted libraries were constructed to generate surface expressed IgG so that peptide-grafted antibodies would not be secreted at high levels and thus activation of receptors on neighboring cells would be minimal. It was unclear prior to developing the reporter assay whether this tethered antibody would still be able to activate the receptor. On the other hand, an advantage of a membrane tethered antibody would be that the majority of the library would be expressing antagonistic peptide-grafted antibodies (i.e. peptide-grafted antibodies that bind to, but do not activate, the receptor) and the secretion of these antagonists in large quantities by the library would have potentially prevented the activation of the receptor by a rare agonist.

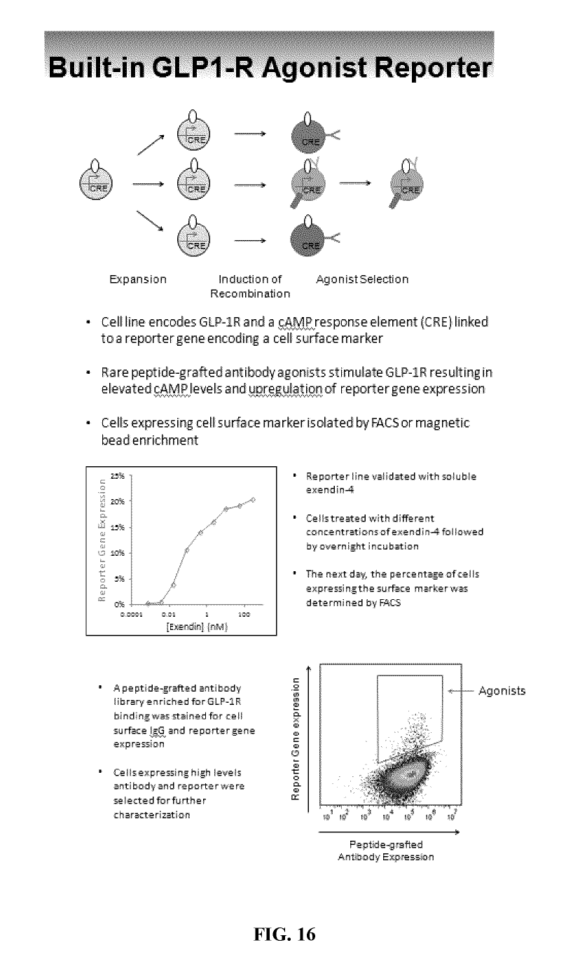

An overview of the development of the reporter cell line is shown in FIG. 16.

The cyclic AMP reporter assay as described in Example 1 below was developed such that activation of the receptor resulted in the expression of the cell surface protein CD19. The reporter was engineered with cAMP responsive elements (CREs) upstream of the CD19 open reading frame. In order to find the most suitable chromosomal location with the appropriate number and ratio of integrated reporter cassettes and GLP1 receptor expression cassettes, the strategy described in Example 1 was employed to empirically identify a useful reporter cell line.

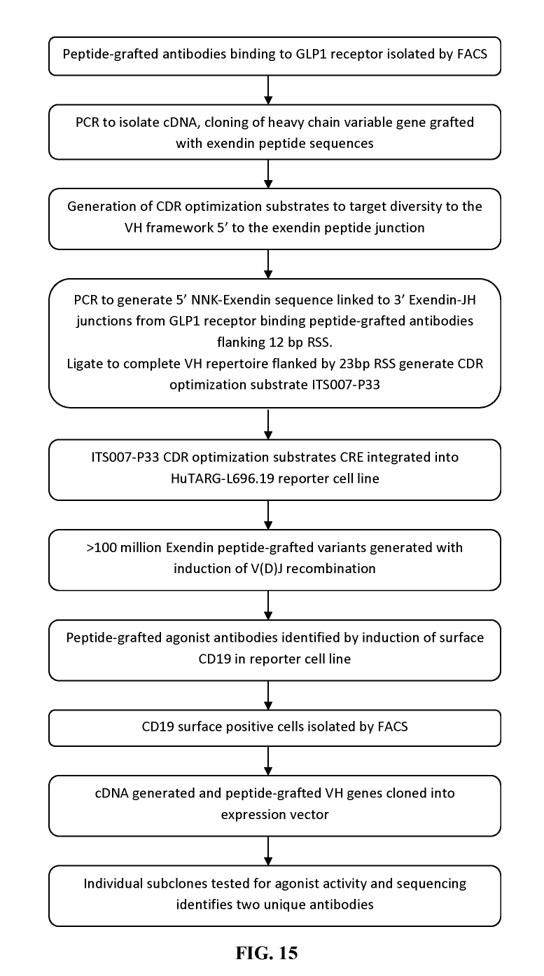

A flow chart outlining the steps described in Examples 1-5 below is provided in FIG. 15.

### Example 1

### Generation of Reporter Cell Line

HuTARG™ cells, which are HEK293 cells engineered to be capable of de novo antibody generation, were used to generate the reporter cell line. HuTARG™ cells have been stably transfected with expression cassettes for RAG1 and RAG-2 and Tdt. RAG-1 expression is inducible as the gene has been placed downstream of a Tet inducible promoter. HuTARG™ cells have also been stably transfected with a plasmid containing a LoxP site for subsequent insertion of V(D)J substrates.

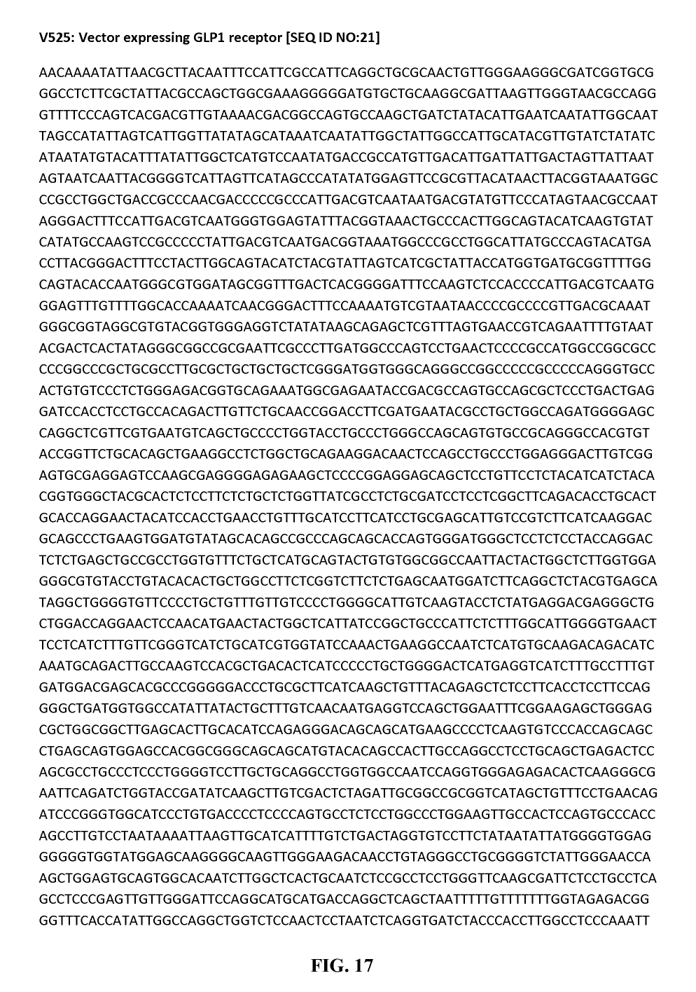

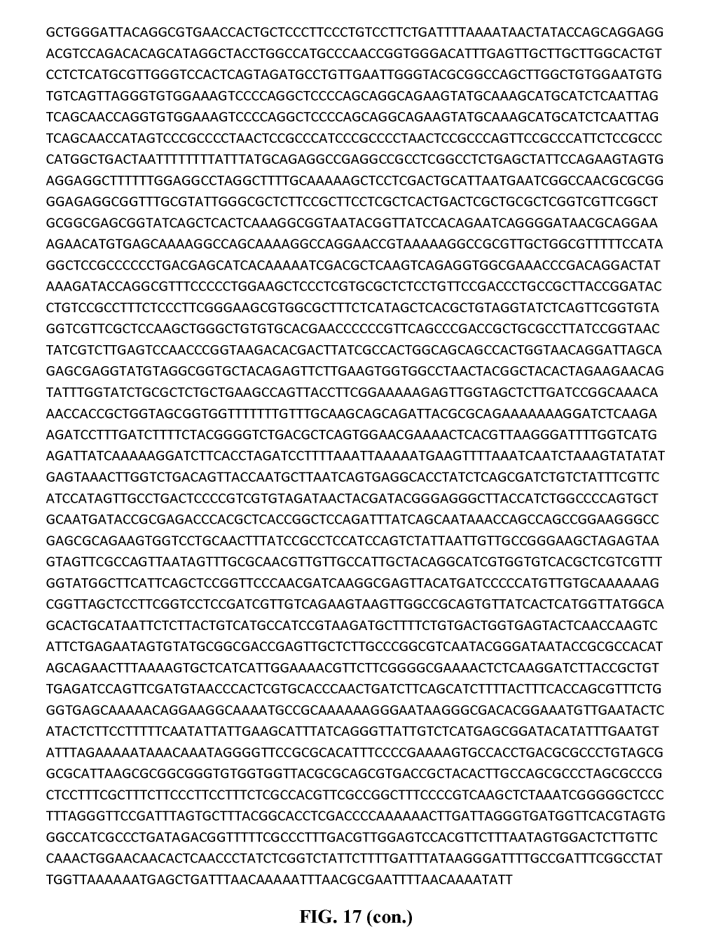

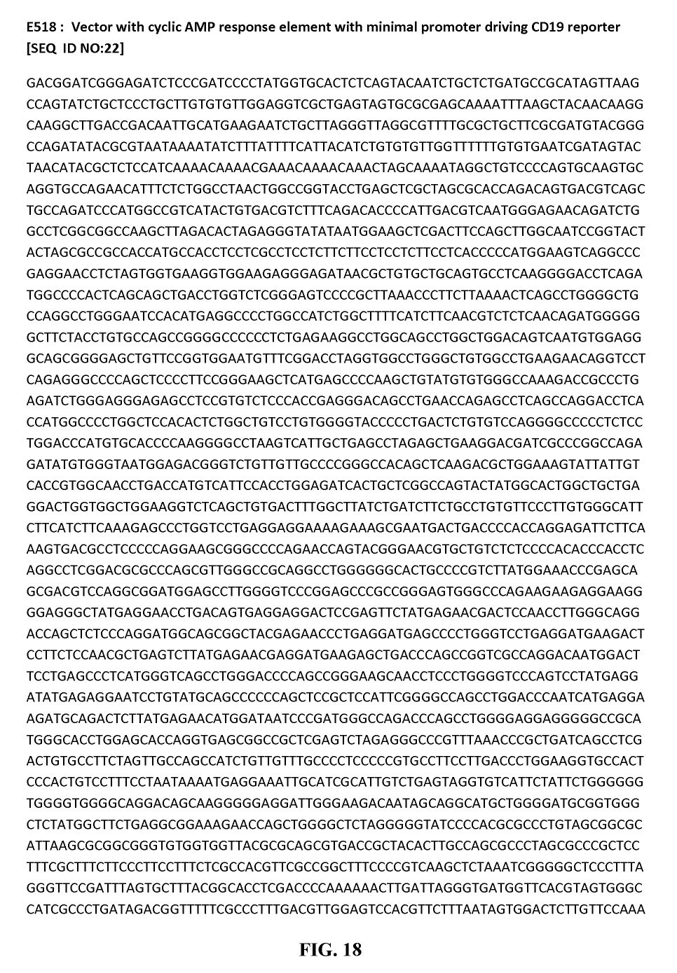

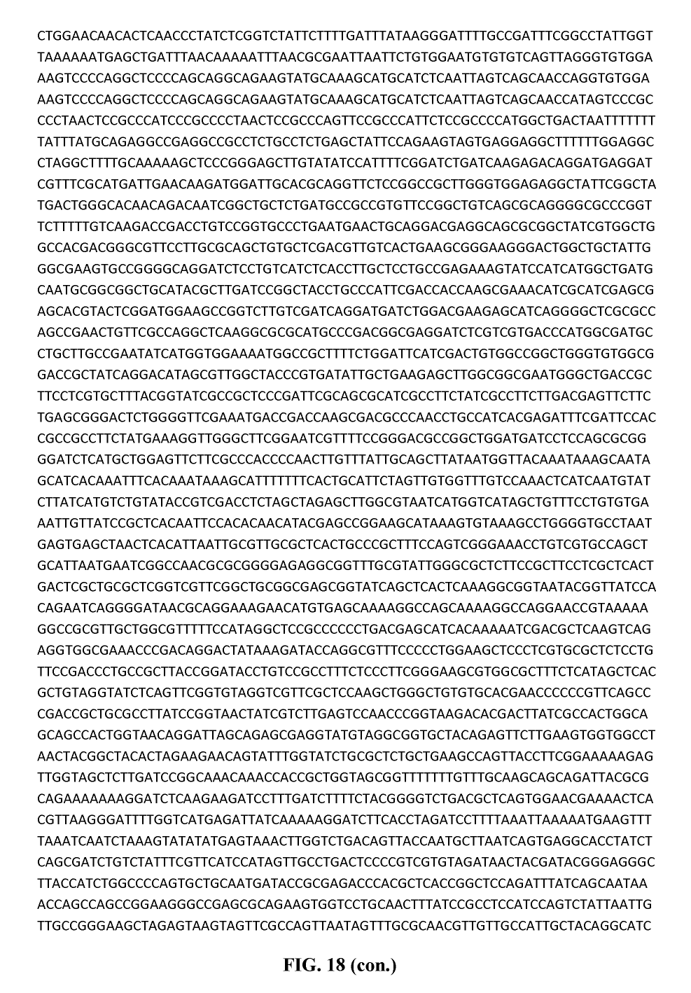

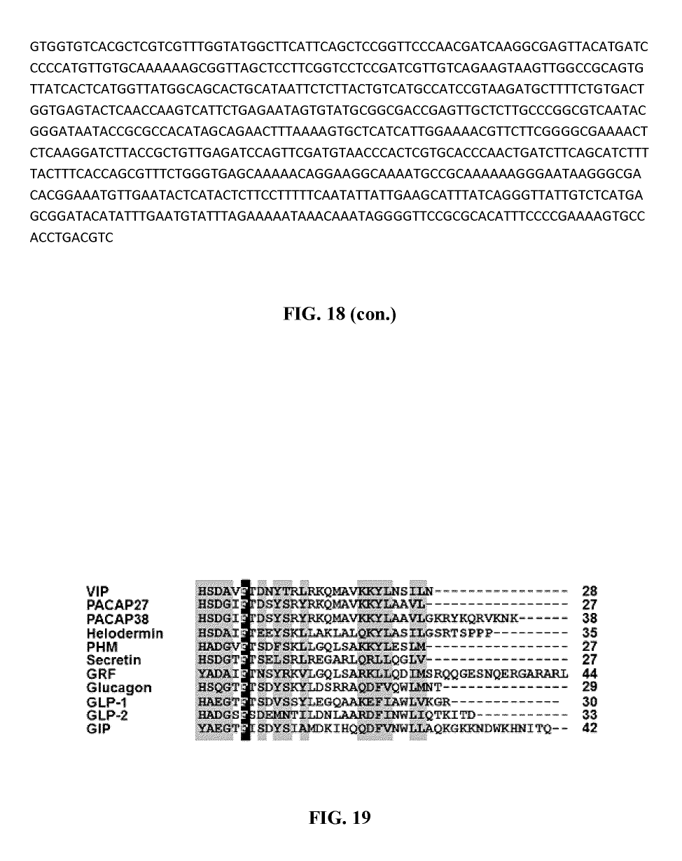

HuTARG™ cells were modified to express the GLP1 receptor as well as express a reporter gene that was activated by cAMP. Approximately 20 million HuTARG™ cells without an integrated V(D)J substrate were transfected with a vector containing CMV driven GLP1R (V525, Origene, SEQ ID NO:21; FIG. 17) and a second vector with a Neomycin resistant marker and a reporter gene (CD19) whose expression is controlled by a cAMP responsive promoter and a (E518; SEQ ID NO:22; FIG. 18). cAMP promoter elements were derived from pGL4.29 (Promega). Twenty micrograms of DNA (50:50 mix) and sixty micrograms of PEI was used for the transfection. Neomycin selection was used to generate a pool of cells stably expressing different ratios of each vector.

Next, to remove stable integrants expressing constitutive CD19, the cell line pool was depleted of CD19 positive cells through two round of bead selection. Briefly, two million stable cells were trypsinized, centrifuged and incubated with an anti-CD19 antibody for one hour. Next, cells were centrifuged, washed and then stained with a biotinylated goat anti-human IgG antibody for an additional hour. The cells were then washed again, resuspended into 500 ul of PBS+2% FBS, incubated with 20 ul of anti-biotin microbeads (Miltenyi Biotech, cat#120-000-900) and then depleted as per manufacturer's suggested protocol using Miltenyi LS magnetic columns.

Depleted cells were subsequently expanded and depleted an additional time using the same protocol except using a Miltenyi LD magnetic column. Next, the twice-depleted cells were treated with 10 nM Exendin (Anaspec) for 24 hours and then enriched for CD19 positive cells using the same staining procedure as above but instead utilizing Miltenyi MS column followed by a MS column and manufacturer's suggested protocol for positive selection. The magnetically isolated cells were expanded and maintained without ligand and subsequently FACS sorted for CD19 negative/GLP1R positive cells. Single cell clones confirmed to have minimal CD19 expression in the absence of ligand were evaluated for induction of CD19 in the presence of 10 nM Exendin and the L696 subclone was selected.

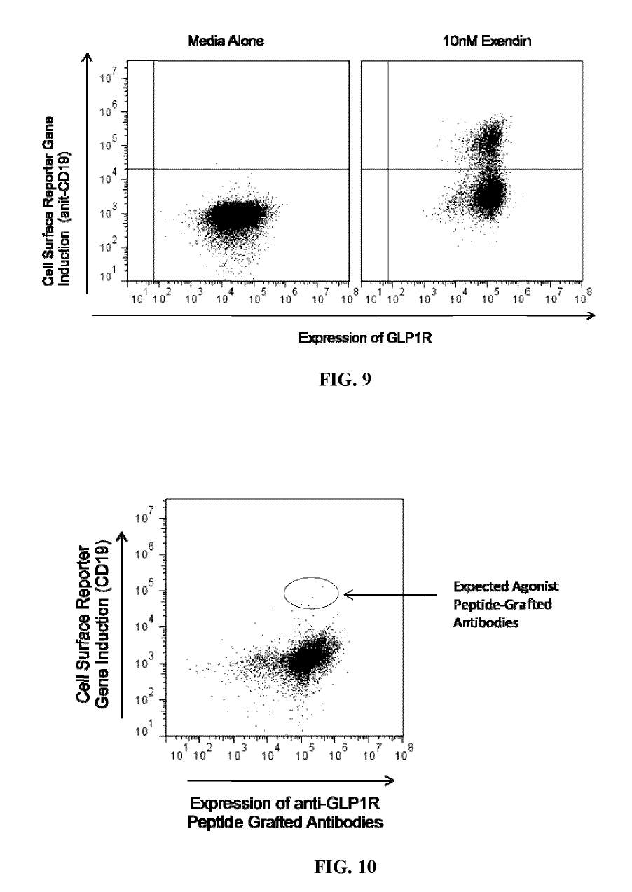

FIG. 9 shows the FACS plots for the L696 cell line in the absence or presence of 10 nM Exendin demonstrating that CD19 expression is specifically induced by the addition of ligand for the GLP1 receptor. Additional experiments were performed with the L696 cell line confirming that cells constitutively expressing a membrane tethered Exendin-Fc fusion protein resulted in CD19 surface expression, thus confirming that a membrane IgG was an appropriate format for screening and that down-regulation of the receptor as a result of constitutive stimulation would not negatively impact the assay. This clone was named HuTARG-696 and was subsequently used to generate de novo peptide-grafted libraries as described in the following Examples.

### Example 2

### Cloning of CDR Diversification Vectors of GLP1-Receptor Binding Peptide-Grafted Antibodies

The cDNA from the Exendin peptide-grafted antibodies (greater than 500) previously generated as described in International Patent Application No. PCT/CA2013/050204 (WO 2013/134881) was amplified using the following primers.

CP360:
[SEQ ID NO: 27]
GAGAGTTTGGATCCCAACTTTCTTGTCCACCTTGGTGTTGC

CP360 is the primer used in the reverse-transcription reaction to generate cDNA.

AL63:
[SEQ ID NO: 28]
GAGAGATTTGGTCTCTATGTCGATCTGATCAAGAGACAGGATAAGGAGCC
MG302:
[SEQ ID NO: 29]
GAGAGAGATTGGTCTCGCCTGAGTTCCACGACACCGTCACC

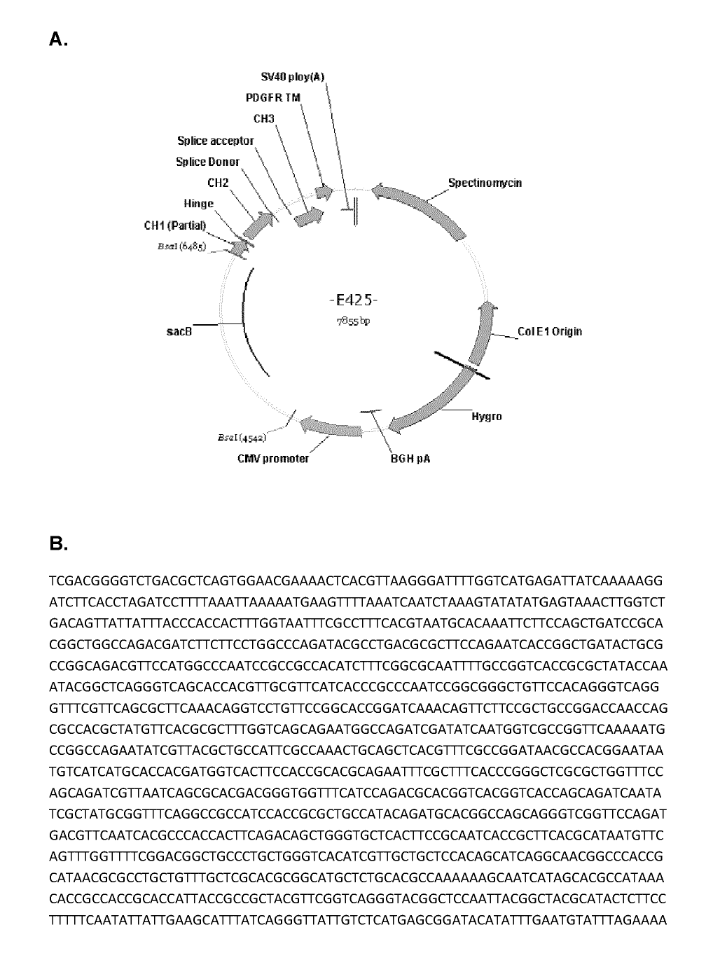

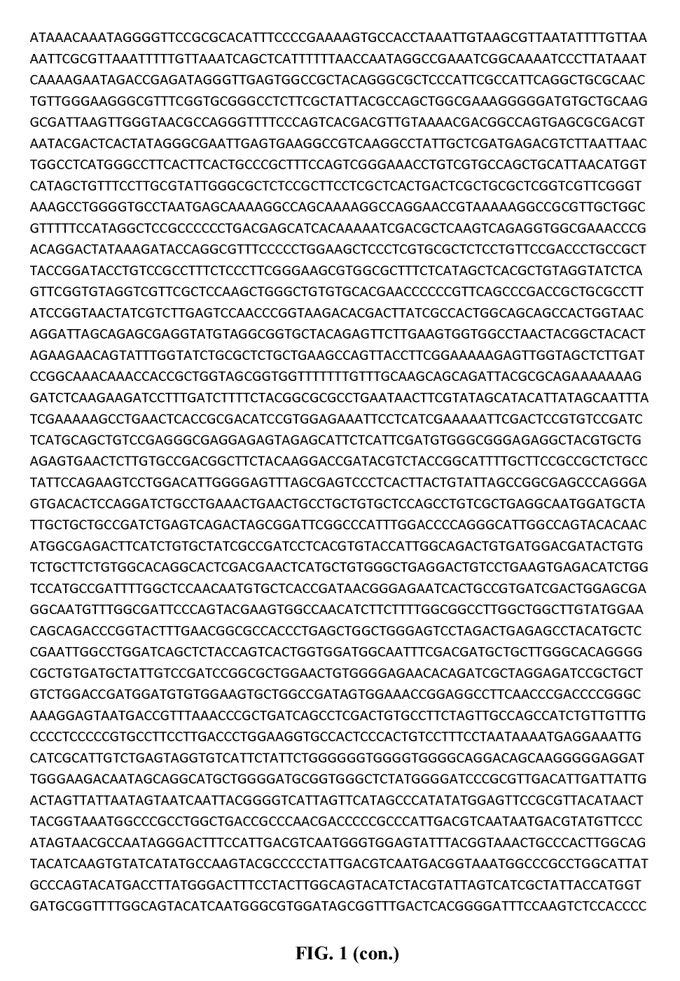

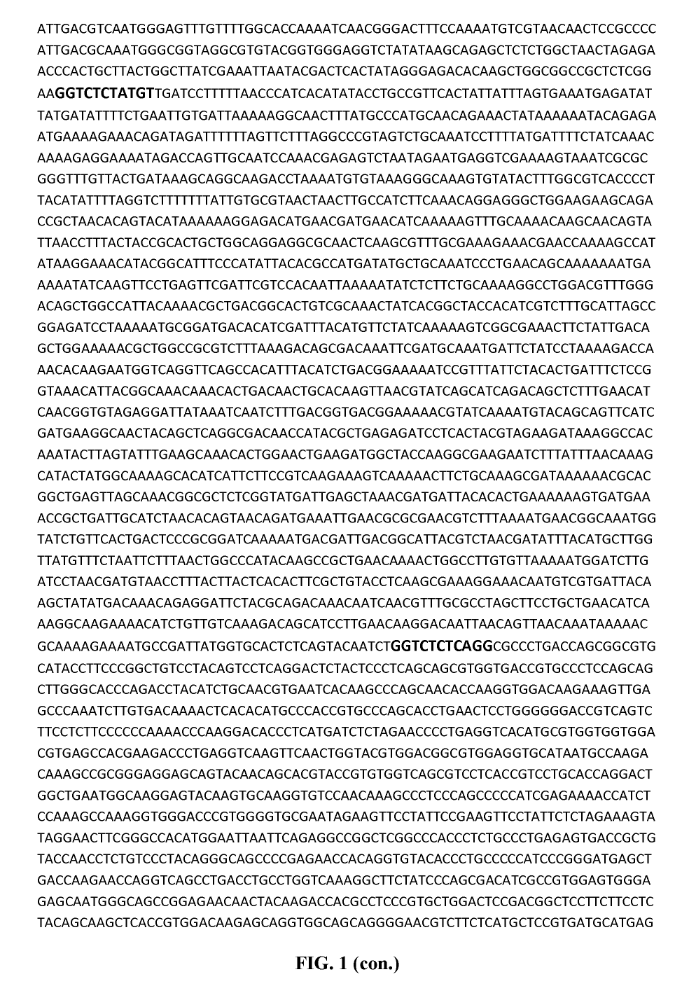

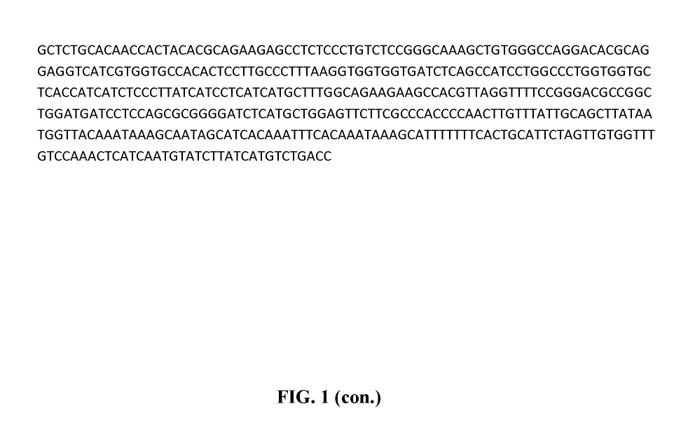

Primer AL63 anneals in the 5′ untranslated region upstream the heavy chain variable region leader sequence found in all the VH gene segments utilized to generate fully human antibodies in International Patent Application No. PCT/CA2013/050204 (WO 2013/134881). All the antibodies generated de novo using the in vitro V(D)J system utilize the same promoter, 5′UTR and leader sequence, so that the primer is universal for all heavy chain variable genes. The MG302 primer anneals in CH1 of the heavy chain and is the reverse primer to amplify the heavy chain cDNA corresponding to the Exendin-peptide-grafted antibodies. The PCR product was generated using the KOD polymerase and was subsequently cloned into vector E425 (see FIG. 1) using the flanking BsaI sites of each primer. E425 contains the appropriate corresponding BsaI sites downstream of a CMV promoter to drive expression of the cDNA and in the heavy chain CH1 to reconstitute the human Ig constant region (FIG. 1). International Patent Application No. PCT/CA2013/050204 (WO 2013/134881) demonstrated that peptide-grafted antibodies that bound to the GLP1 receptor could be generated. Evaluation of individual clones isolated from the de novo Exendin peptide-grafted antibodies for GLP1 receptor agonist activity were, however, negative. The pool of approximately 5000 clones representing greater than 100 binders was subsequently used as the template for PCR using primers:

ET70:
[SEQ ID NO: 30]
CTACGAGGTCTCACTGTGNNKCATGGCGAGGGCACCTTCACCTCCGACCTG
TCCAAAC
ET71:
[SEQ ID NO: 31]
TTAAGAGGTACCCTCGAGCAGACATGATAAGATACATTGATGAGTTTGGAC
AAAC

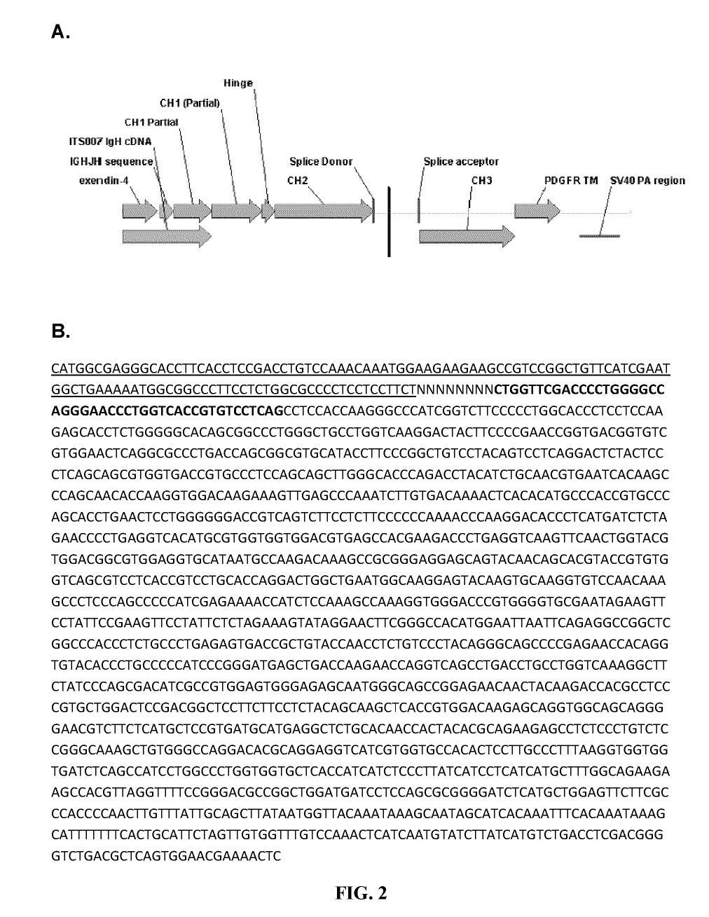

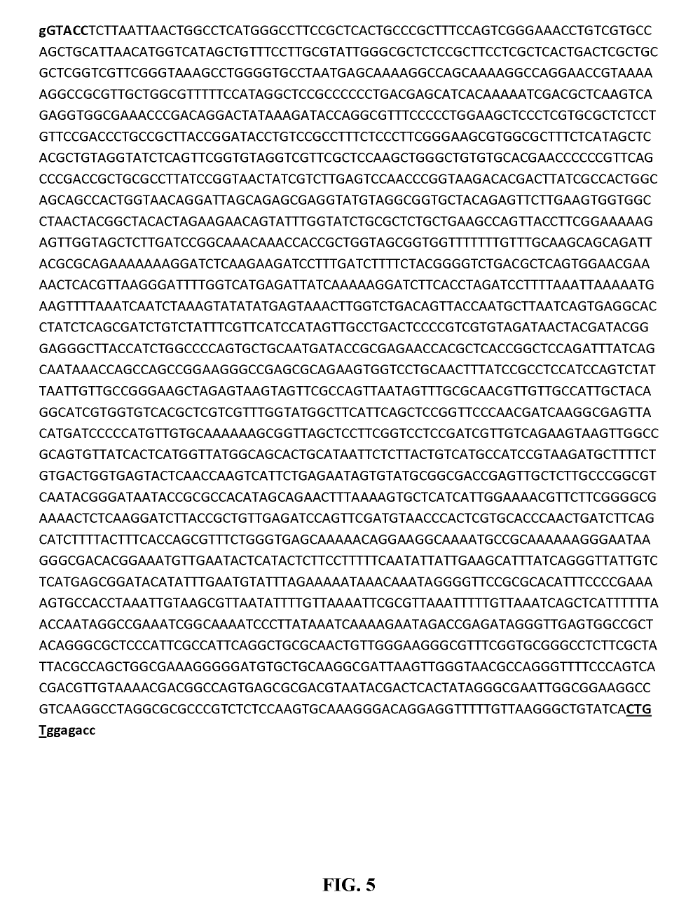

The use of the ET70 and ET71 primers generated a PCR product that extended 3′ from the histidine codon at the start of the sequence encoding Exendin to the SV40 PolyA (see FIG. 2). The ET70 primer includes a BsaI cloning site and three degenerate nucleotides upstream of the Exendin open reading frame and provides additional substrate diversity; it is equivalent to increasing the number of D segments available for the recombination reaction. The 3′ end of Exendin was represented by all the GLP1 receptor binding clones that were isolated; these 3′ junctions were previously shown to support binding to the receptor. The ET71 primer has a KpnI tail for cloning. The pool of Exendin-based sequences with degenerate 5′ ends and 3′ ends derived from receptor binding peptide-grafted antibodies was cloned into vector V610 (see FIG. 5) as a pool of BsaI-KpnI fragments to generate a pool referred to as ITS007-P31 and which represented approximately 148,000 different subclones.

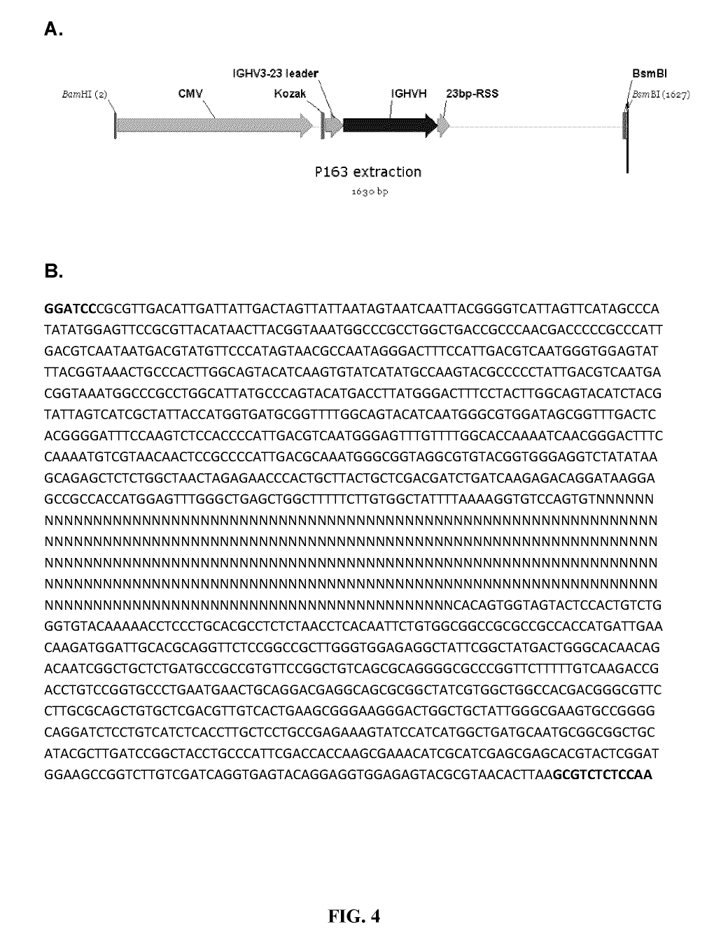

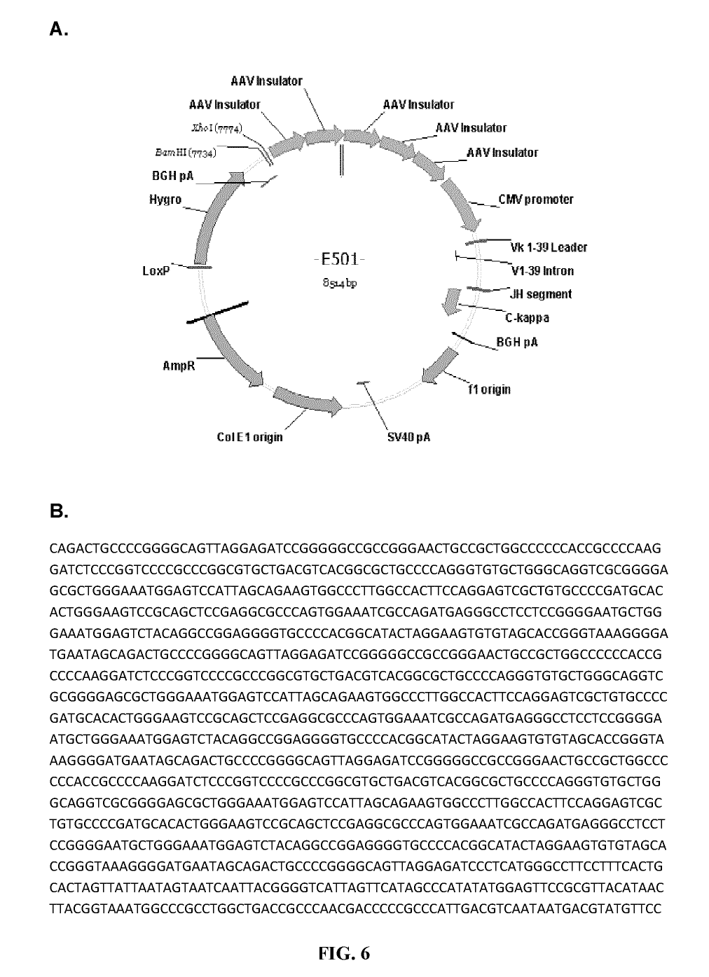

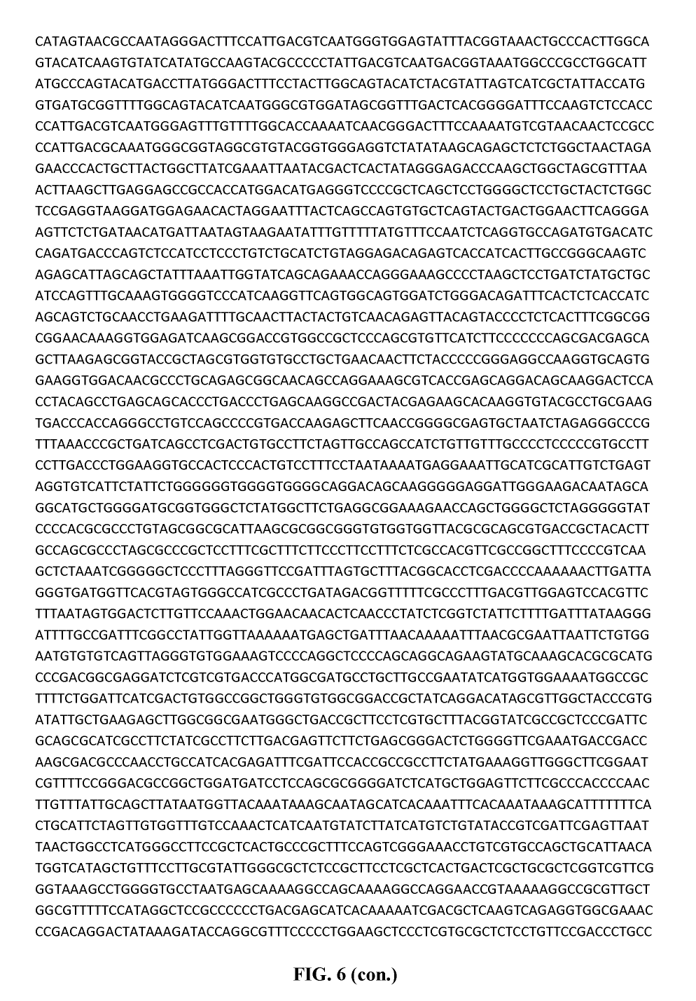

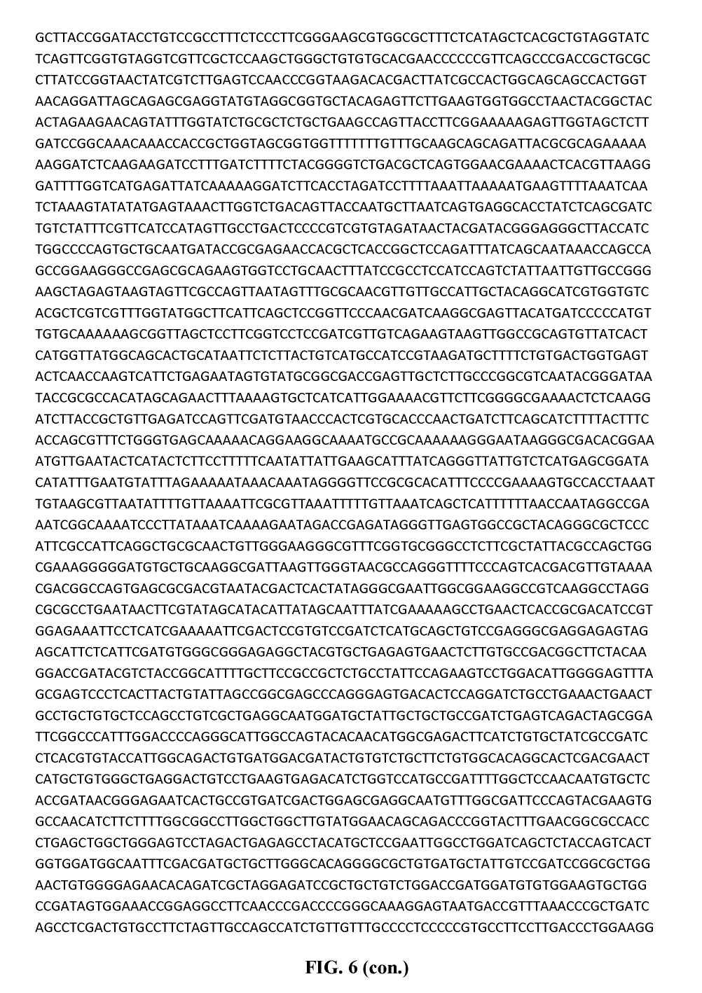

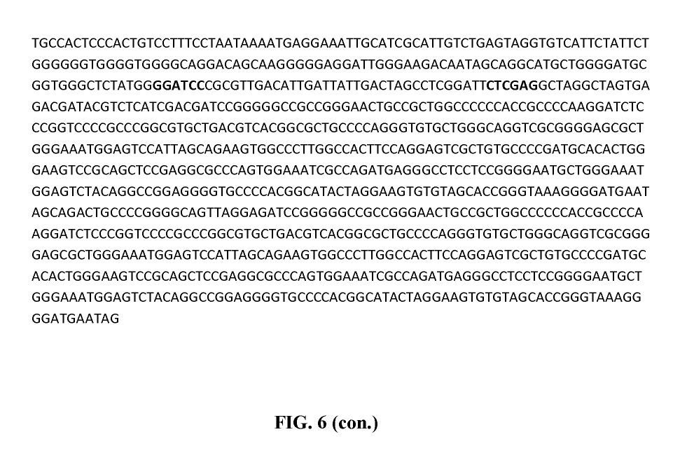

The ITS007-P31 pool was digested with BsmBI and XhoI and co-ligated with the BsmBI and BamHI fragment from P163 (see FIG. 4) into the BamHI-XhoI sites of vector E501 (see FIG. 6), which is a V(D)J substrate that also expresses a VK1-39-based light chain. The E501 vector additionally contains AAV insulators between the V(D)J recombination substrate and the light chain expression cassette. E501 also contains a LoxP site in-frame with a hygromycin resistance gene for selection of Cre-mediated integration. In the resulting pool of V(D)J substrates, each contained a 5′ NNK sequence in the context of 3′ flanking sequences isolated from GLP1 receptor binders. The pool was referred to as ITS007-P33.

The ITS007-P33 pool was Cre-integrated into the HuTARG-696 reporter cell line to a chromosomal site previously shown to support Cre recombination, V(D)J recombination and high expression. The integrated LoxP site contains an upstream CMV promoter and ATG/Kozak required for expression of the hygromycin resistance marker in ITS007-P33; this measure was introduced to minimize random integration of ITS007-P33. Two 10-cm plates of the HuTARG-L696 reporter cell line were seeded 24 hour prior to transfection in 12 mls of DMEM. 17.3 ug of the ITS007-P33 DNA and 1.9 ug of the V503 DNA was used per transfection and was added to 2.4 ml of PRO293 serum free media. 2.4 ml of PRO293 containing 57.6 ul of 1 mg/ml PEI was added to the DNA, vortexed and allowed to complex for 20 minutes prior to being added to the cells. Forty-eight hours post-transfection, Cre-mediated integrants were selected by the addition of Hygromycin B (100 ug/ml). Hygromycin selection was allowed to proceed for 10 days. The Cre-integrated pool of V(D)J substrates was named HuTARG ITS007-L31.

### Example 3

### Induction of V(D)J Recombination

A 10-cm plate of the HuTARG ITS007-L31 cells was seeded for V(D)J induction (approximately 24 million cells). Induction was essentially as described in International Patent Application No. PCT/CA2013/050204 (WO 2013/134881) with the following modifications. Vector V429 expressing TdT was transfected into the cells and 24 hours following transfection, the 10-cm plate was split into a 175 cm² flask at which time RAG-1 was induced by adding 1 ug/ml Tetracycline. Tet-inducible RAG-1 was generated in the HuTARG cell line using the Tet-On system from Invitrogen (Carlsbad, Calif.). V(D)J induction was allowed to proceed for 5 days at which point the flask was trypsinized and seeded into a suspension shake flask at approximately 1.5 million cells/ml resulting in a total induced cell population of approximately 150 million cells. Puromycin (1 ug/ml) was added to the shake culture (to select for in-frame peptide-grafted antibody sequences) and the cells were cultured for 2 days. Media was removed and replaced, and the culture was re-seeded at 1.5 million cells per ml and puromycin selection continued for another 3 days. Cells were subsequently harvested and plated back to adherent flasks where they were maintained under selection until used.

### Example 4

### Screening for Agonists

The puromycin-selected HuTARG ITS007-L31 cells from Example 3 were confirmed to have surface IgG. The HuTARG ITS007-L31 library was then analyzed for the presence of surface-expressed CD19, which would indicate that the GLP1R was activated.

Approximately 1 million recombined HuTARG ITS007-L31 cells that surface-expressed anti-GLP1R peptide-graft antibodies were stained with anti-CD19-PE conjugated antibody (1 ug/ml) and an anti-IgG Fc Alexa-647 conjugated antibody (1 ug/ml). The results of FACS analysis of these cells is shown in FIG. 10 and demonstrates that a large fraction of the library generated peptide-grafted antibodies that bind to the GLP1 receptor (X-axis), but only a small fraction of these cells were CD19 positive (Y-axis) and represented potential agonists. The CD19 positive/IgG positive population was FACS sorted and the cDNA was isolated from 1000 positive cells. The isolated cDNA was cloned into a expression vector to express the isolated heavy chain.

The expression vectors comprising the sequences for the isolated heavy chain antibodies were co-transfected into the HuTARG-L696 reporter cell line with a second vector expressing the VK1-39 kappa light chain. 48 hours post-transfection, cells were stained for surface CD19 expression. A number of positive clones were identified. On sequencing, it was determined that these positive clones represented just two different antibodies with agonist activity (Table 4; bold sequences represent the grafted exendin peptide, italicized sequences represent sequences from the VH or JH gene segment).

TABLE 4
Sequences of Isolated Agonist
Peptide-Grafted Antibodies

| | | Sequences proximal | SEQ |
|---|---|---|---|
| ID | IGHV | to exendin (in bold) | ID NO |
| ITS007- | 2-70 | CARHGEGTFTSDLSKQMEEEAVRLFIEWLKN | 32 |
| V105 | | GGPSSGAPPPSSLQDDAFDIWGQGTMVTVSS | |
| ITS007- | 3-9 | CARDERHGEGTFTSDLSKQMEEEAVRLFIEW | 33 |
| V107 | | LKNGGPSSGAPPPSVAAFDIWGQGTMVTVSS | |

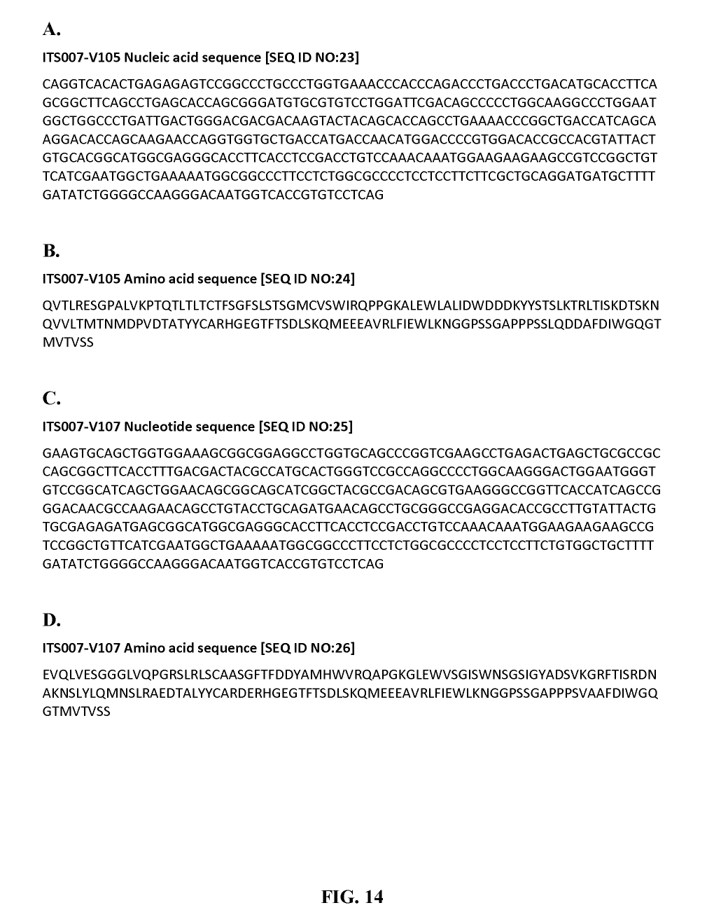

The two peptide-grafted antibodies utilize different VH gene segments (VH2-70 or VH3-9) and also differed at both the VH-5′ Exendin junction and the 3′ Exendin-JH junction in both the length and composition of amino acids. The two sequences interestingly, however, both contained an arginine flanking the histidine at the N-terminus of the Exendin sequence. Both sequences utilized the JH3 gene segment. The complete nucleotide and amino acid sequences for ITS007-V105 and ITS007-V107 are provided in FIG. 14 (SEQ ID NOs:23-26).

Preliminary mutagenesis studies in which the amino acid at the −1 position relative to the His in Exendin (i.e. Arg in the two sequences shown in Table 4) was mutated to other amino acids were conducted. Appreciable agonist activity in the above-described reporter cell line assay was seen only with Arg or Lys at this position.

### Example 5

### Activity of Agonists in Absence of Light Chain

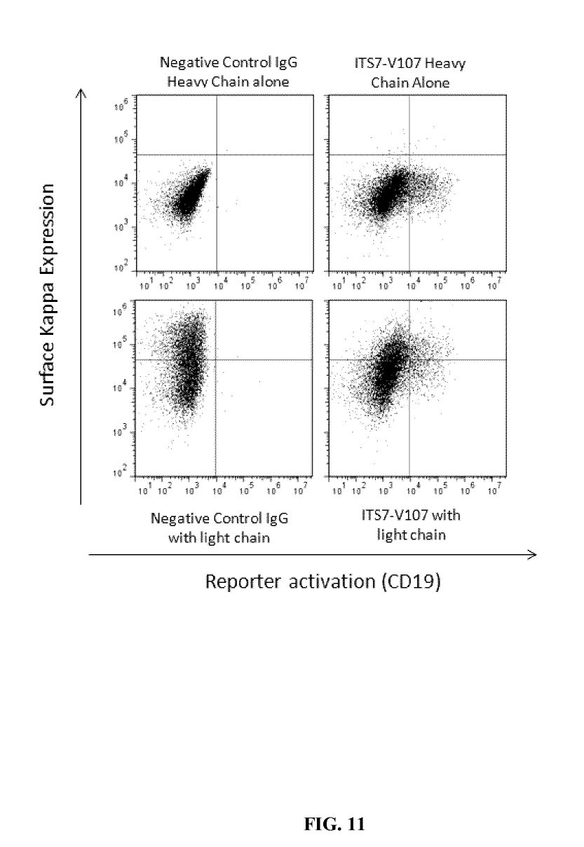

In control experiments, agonist activity from the two antibodies (ITS007-V105 and ITS007-V107) was also observed in the absence of light chain. FIG. 11 shows cells transfected with a human heavy chain (upper left), an intact human antibody with heavy chain and light chain (lower left), ITS007-V107 peptide-grafted heavy chain (upper right) or an intact peptide-grafted antibody comprising ITS007-V107 peptide-grafted heavy chain and a human light chain (lower right). The cells were co-stained for human kappa and for surface-expressed CD19.

The left-hand panels demonstrate that the heavy chain only transfection is negative for human kappa chain (upper panel) while the intact antibody in the lower left panel has cells with human kappa on the surface as an intact antibody. Both the left-hand panels are CD19 negative and reflect that the control antibody does not induce cAMP in the reporter cell line.

The right-hand panels show that only the lower panel has surface kappa expression but that both the right-hand panels, i.e. with and without the presence of a light chain, show CD19 staining, indicating that both the intact peptide-grafted antibody and the peptide-grafted heavy chain alone are capable of stimulating the GLP1 receptor.

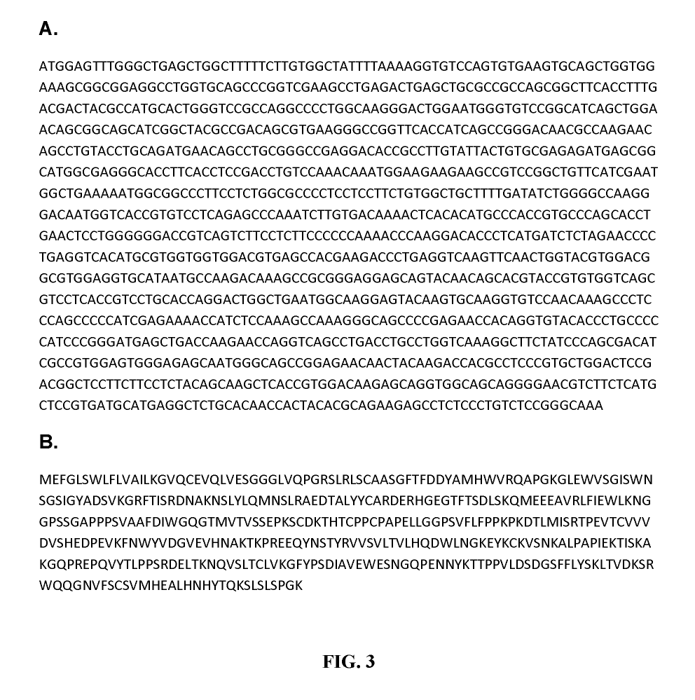

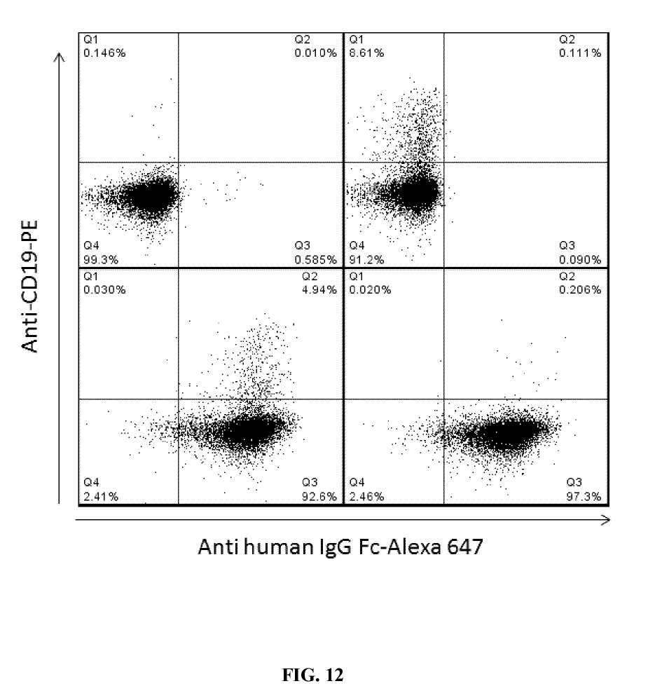

The peptide-grafted antibody, ITS007-V107, was subsequently cloned and expressed as a single-domain VH-IgG antibody without CH1 or a light chain and it was confirmed that the peptide-grafted heavy chain was functional in this format as well (FIG. 12). This peptide-grafted heavy chain, named ITS007-V125 (see FIG. 3, SEQ ID NOs:50 and 51), was transfected into HEK293 cells and supernatant was collected 5 days post transfection. Expression levels were greater than 5 ug/ml. The supernatant was then assayed on the HuTARG-L696 reporter line. FIG. 12 demonstrates that the reporter cell line alone does not have significant CD19 surface expression (upper left panel). The reporter line incubated with 50 nM Exendin for 48 hours resulted in approximately 8% of the cells showing various levels of CD19 expression (upper right panel). Incubation of the ITS007-V125 supernatant with the reporter cell line for 48 hours at a final concentration of 1 ug/ml resulted in approximately 5% CD19 positive cells (lower left panel). In the same experiment, the cells were stained for anti-IgG demonstrating that the ITS007-V125 single-domain antibody was binding to the GLP1R on the reporter cell line.

A control single-domain VH-IgG antibody, ITS007-V129, was also generated. The control antibody is identical to ITS007-V125 except for a histidine to alanine mutation corresponding to the first amino-acid in the Exendin peptide. This mutant antibody demonstrated similar binding to the reporter cell line but minimal agonist activity (lower right panel of FIG. 12). This result is consistent with mutagenesis studies of Exendin and GLP1 that indicate the central role of the amino terminal histidine in agonist activity.

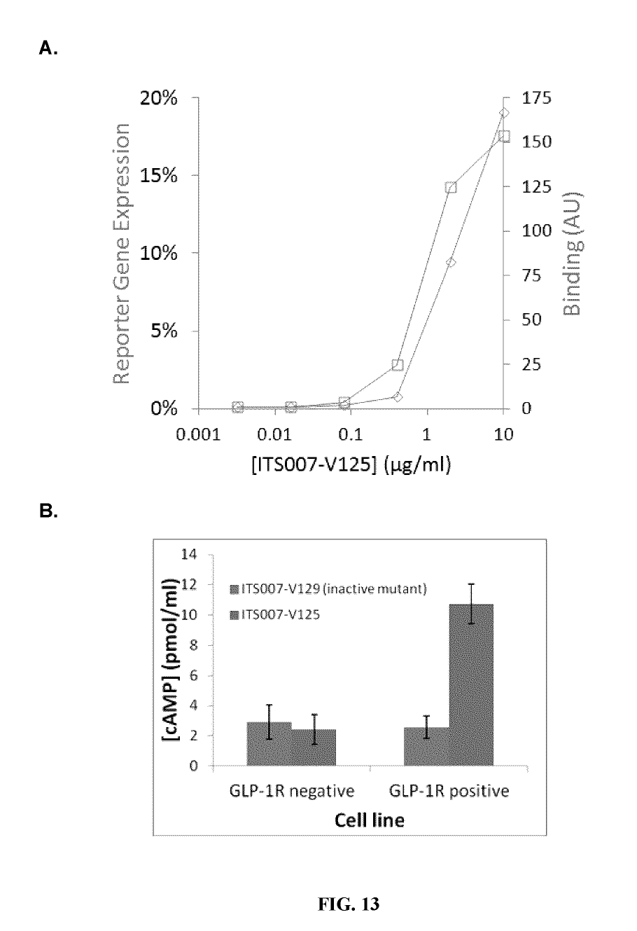

ITS007-V125 was cloned into a secretion vector, expressed and purified. The reporter cell line was treated with different concentrations of ITS007-V125 followed by overnight incubation. The next day, binding and the percentage of cells expressing the surface marker were determined by FACS. The results are shown in FIG. 13 and confirm that the peptide-grafted heavy-chain ITS007-V125 has GLP1R agonist activity.

### Example 6

### Generation of Peptide-Grafted Glucagon Receptor Agonists in a Heavy Chain Only (HCAb) Format

Experiments with the GLP1 agonists generated as described above indicated that the agonist activity did not require the light chain and the peptide-grafted heavy chain was sufficient for all activity consistent with the peptide sequences being the primary element responsible for function. Accordingly, peptide-grafted glucagon receptor agonists were generated in a heavy chain only (HCAb) format (i.e., including VH+CH2 and CH3, but not CH1).

A single chain format offers a number of advantages with regards to the generation of potential therapeutics. For example, because it is a single chain it provides the opportunity to generate a more modular system of combining different specificities. A single chain also may provide more convenient manufacturing opportunities. Not all heavy chains are expressed well in the absence of the light chain in an in vitro mammalian system. A unique set of VH gene segments, different from what has previously be reported, was identified that performs well as a single chain in a mammalian cell. Specifically, the following VH gene segments were identified as expressing well in the HCAb format: VH1-8, VH3-11, VH3-15, VH3-20, VH3-30, VH4-34.

The repertoires for grafting the glucagon peptides were generated using the above-noted restricted VH gene set. Four different sets of V(D)J diversification substrates were generated as shown below. Two utilized the glucacon peptide sequence (SEQ ID NO:10) and two used the oxyntomodulin (oxN) sequence (SEQ ID NO:34). One set of diversification substrates had three nucleotides (TCC, corresponding to a serine) flanking the 5′ RSS and three nucleotides (GTG corresponding to valine) flanking the 3′ RSS. The second set included 9 additional nucleotides of 3 sets of NNK nucleotides on each side of the peptide sequences.

ITS011-P11:
12 bp-RSS TCC NNKNNKNNK Glucagon peptide seq
NNKNNKNNK GTG 12 bp-RSS
ITS011-P12:
12 bp-RSS TCC Glucagon peptide seq GTG 12 bp-RSS
ITS011-P13:
12 bp-RSS TCC NNKNNKNNK OXN peptide seq NNKNNKNNK
GTG 12 bp-RSS
ITS011-P14:
12 bp-RSS TCC OXN peptide seqGTG 12 bp-RSS

The nucleotide sequence encoding the glucagon peptide employed was:

[SEQ ID NO: 35]
CATTCACAGGGCACATTCACCAGTGACTACAGCAAGTATCTGGACTCCAGG
CGTGCCCAAGATTTTGTGCAGTGGTTGATGAATACC

The nucleotide sequence encoding the oxyntomodulin peptide employed was:

[SEQ ID NO: 36]
CATTCACAGGGCACATTCACCAGTGACTACAGCAAGTATCTGGACTCCAGG
CGTGCCCAAGATTTTGTGCAGTGGTTGATGAATACCAAGAGGAACAGGAAT
AACATTGCC

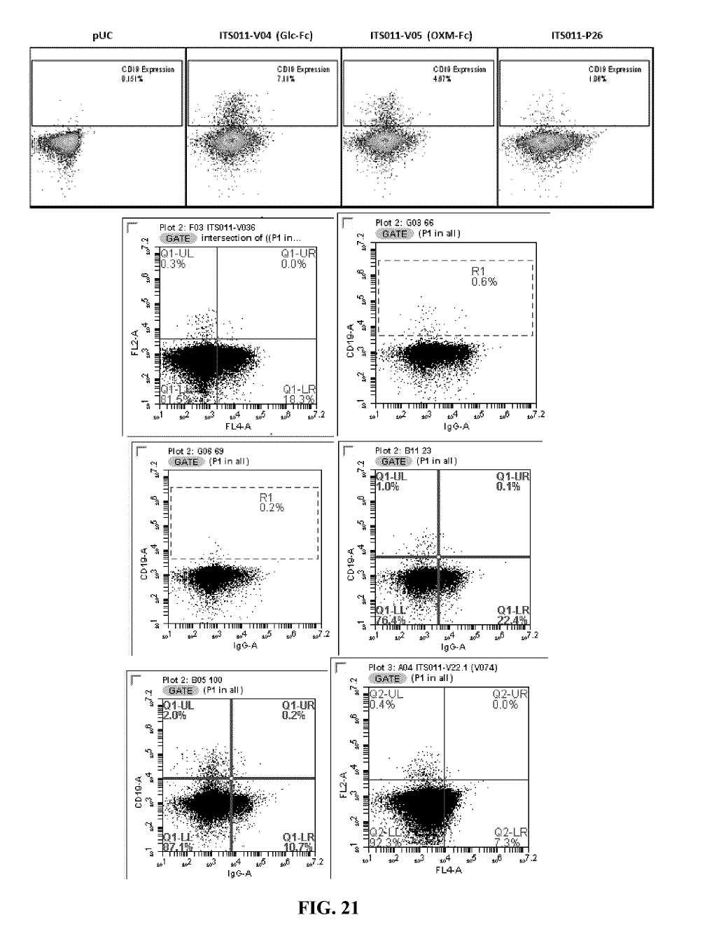

The peptide-grafting V(D)J tripartite substrates were CRE integrated into a host HEK293 cells that contained a single chromosomal LoxP site. The site had been selected to be competent for V(D)J recombination. The HEK293 line also was engineered for assaying for cAMP induced reporter function similar to the GLP1 reporter cell line in the preceding Examples except the glucagon receptor was used instead of the GLP1 receptor. The activation of the receptor by the peptide ligand was shown to activate expression of a cell surface marker (see FIG. 21; Glc-Fc control and OXN-Fc control).

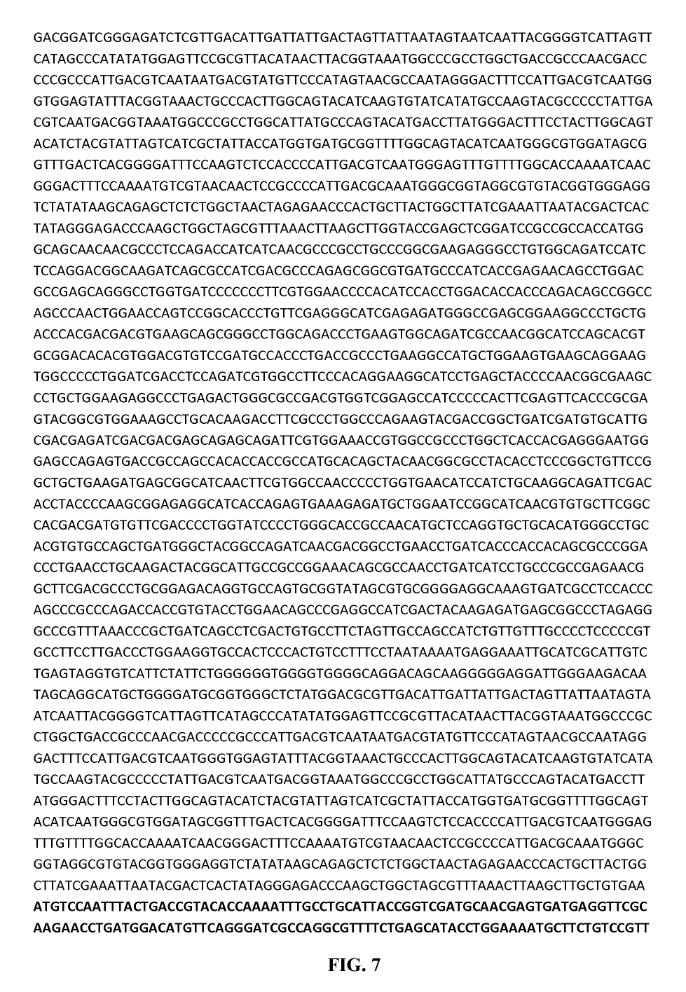

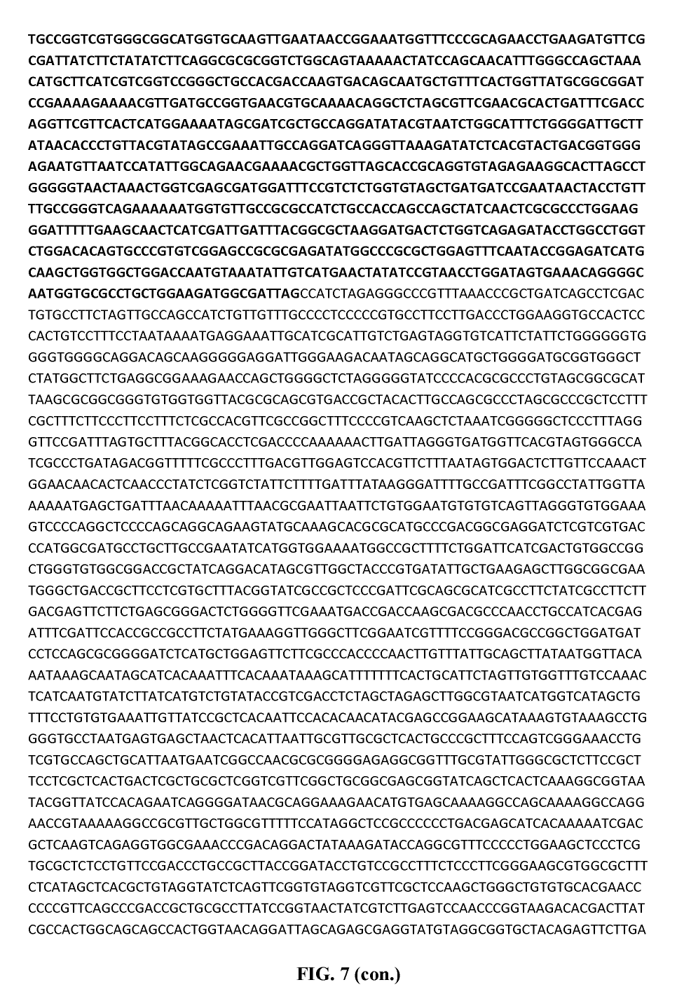

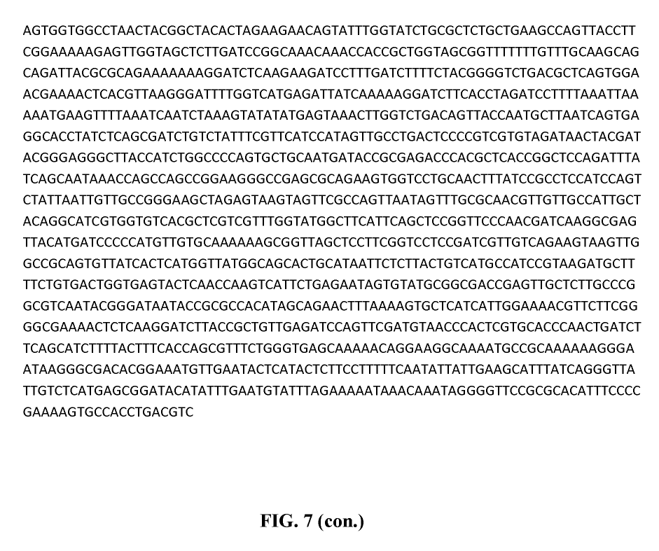

The substrate was engineered to include two FLAG tag sequences in-frame with the LoxP site and hygromycin allowing for magnetic enrichment and/or hygromycin selection for CRE integration events. CRE integration was performed by transfecting the V(D)J substrate and a plasmid expressing the CRE recombinase (V503—see FIG. 7) at a ratio of 10:1. Transfected cells were expanded to approximately 200 million cells and then magnetically enriched using anti-FLAG antibodies and magnetic columns from Miltenyi. CRE pools of approximately 10-50,000 clones were generated and used for subsequent experiments.

V(D)J induction was performed as described for GLP1 in the Examples above. Cells expressing agonist peptide-grafted antibodies were identified by the expression of the cAMP responsive cell surface marker. Cells were magnetically enriched and then subsequently FACS sorted. cDNA was isolated from the FACS sorted cells and individual clones were assessed for activity as described below.

PEI Max Transfections:

One day prior to transfections, ITS011-L05.13 cells were trypsinized and seeded 1:3 into a 24-well plate. Next day, cells were transfected as shown in Table 5.

TABLE 5

| | | | Stock [DNA] | ug | ul | # Wells per |
|---|---|---|---|---|---|---|
| Conditions | Plasmid | | ug/ul | DNA | DNA | Sample |
| 1 | pUC | Empty Plasmid | 0.500 | 0.800 | 1.6 | 1 |
| 2 | ITS011-V04 | Glc-Fc Secreted | 0.317 | 0.800 | 2.5 | 1 |
| 3 | ITS011-V05 | OXM-Fc | 0.306 | 0.800 | 2.6 | 1 |
| | | Secreted | | | | |
| 4 | ITS011-P26 | ITS011-L31 | 0.464 | 0.800 | 1.7 | 1 |
| | | cDNA Pool | | | | |
| 5 to 18 | ITS011-V96 | Isolated clones | 0.100 | 0.800 | 8.0 | 1 |
| | to -V109 | from ITS011- | | | | |
| | | P26 | | | | |

The above indicated amount of DNA for each transfection condition was each added to 50 ul of sf Pro293 media and incubated at room temperature for 5 minutes. 64 ul of 1 mg/mL PEI Max was added to sf Pro293 in 1 mL final volume and incubated for 5 minutes at room temperature. 50 ul of PEI mix was added to each plasmid prep and incubated at room temperature for 20 minutes. Each 100 ul transfection mix was added drop-wise to a well. Cells were returned to culture at 37° C. and were stained the next day.

CD19-IgG Staining:

The day after transfection, cells were stained as follows. Media was removed from each well and each well was gently rinsed with PBS. PBS was aspirated off and each well was trypsinized with 75 ul of 1× trypsin and cells briefly incubated at 37° C. Each well was collected with 500 ul of complete DMEM and spun down to remove supernatant. Each pellet was resuspended with 100 ul of Mouse αCD19-PE (1:200) and Goat αHumanFc647 (1:5000) in FACS Buffer, and samples were incubated for 1 hour at room temperature then each spun down to remove supernatant. Each sample was washed once with 300 ul of PBS and wash was removed via spinning down, then each sample was resuspended with 250 ul of PBS and analyzed via the Accuri (Beckton Dickinson, New Jersey).

### Results

Isolated cDNA was tested for agonist activity. Background agonist activity represented by expression of the cell surface reporter (CD19) activity was approximately 0.1% across experiments. Agonists displaying different amounts of activity were isolated (see FIG. 21).

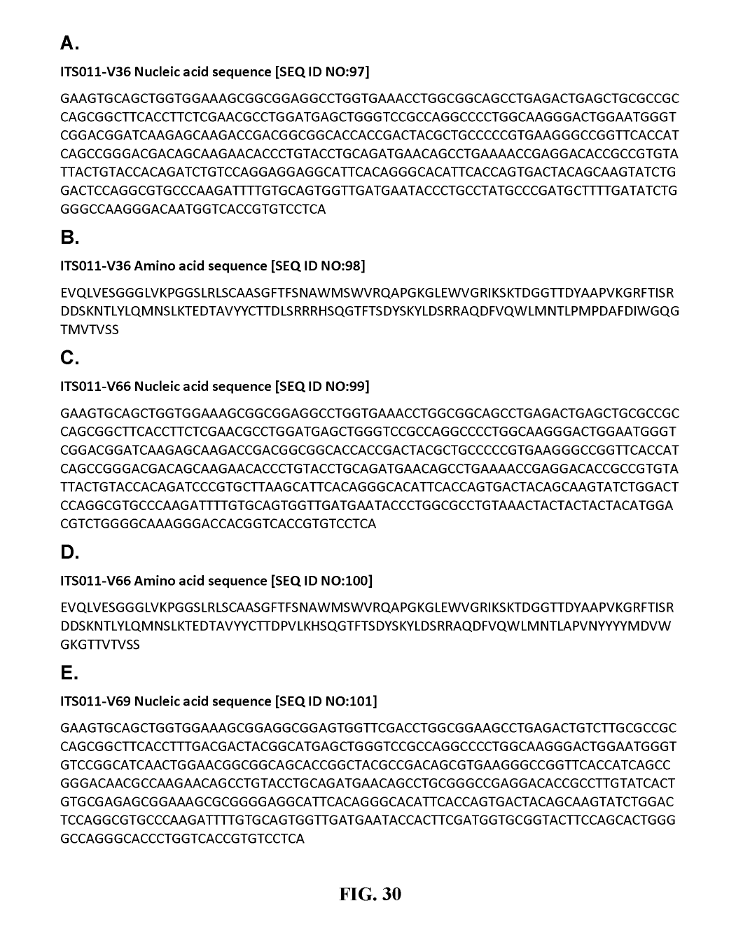

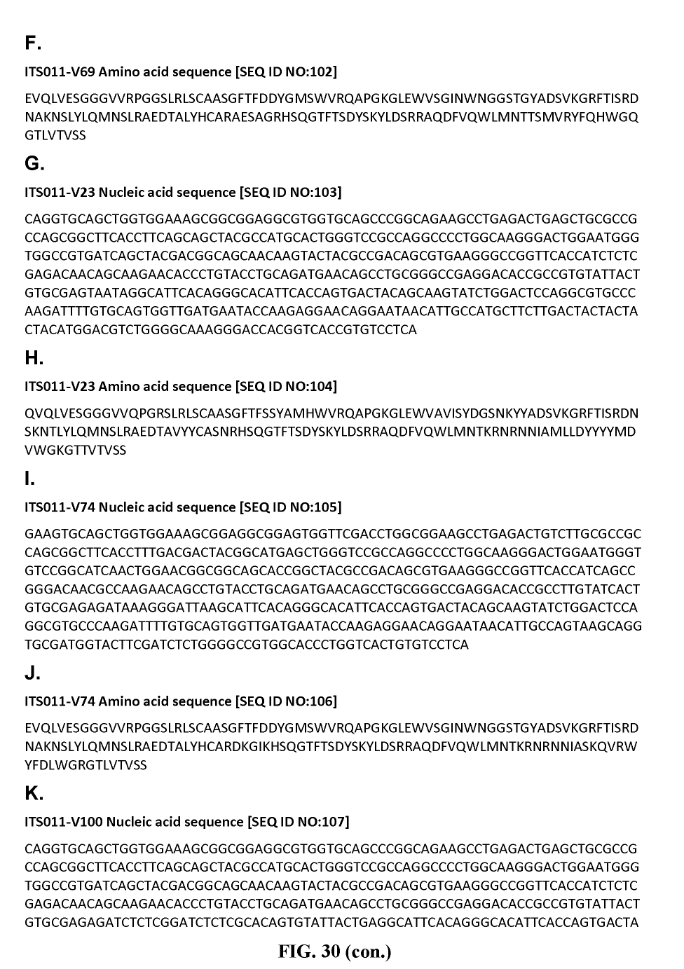

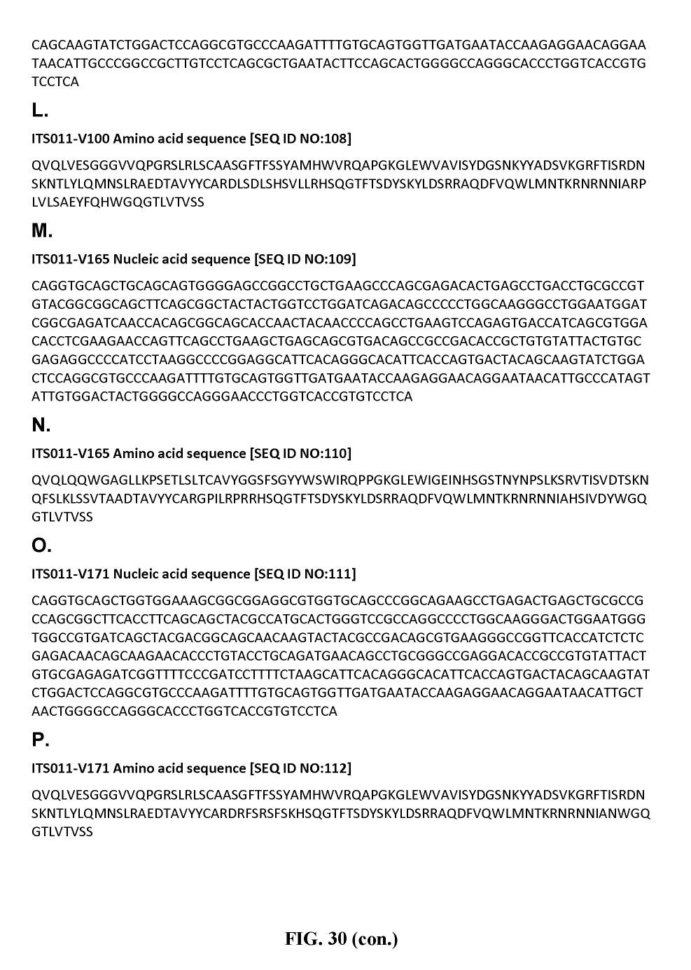

The sequences of representative agonist clones are shown in Table 6 and FIG. 30. Strikingly, the clones with agonist activity had either the amino acid arginine or lysine following the natural amino terminal histidine in the glucagon and oxyntomodulin peptide sequences, as was observed for the GLP1 agonists in the preceding Examples. In contrast, the carboxy terminus junction of the peptides and JH segments did not show any obvious sequence conservation.

TABLE 6
Sequences of Representative
Glucagon Agonist Clones

| | | | | |
|---|---|---|---|---|
| | | | | SEQ |
| | VH | | JH | ID |
| ID | Gene | Sequence* | Gene | NO |

Glucagon

| | | | | |
|---|---|---|---|---|
| ITS011- | 3-15 | CTTDLSRR [symbol] HSQGTFTSDYSKYL | 3 | 37 |
| V36 | | DSRRAQDFVQWLMNTLPMP | | |
| ITS011- | 3-15 | CTTDPVL [symbol] HSQGTFTSDYSKYLD | 6 | 38 |
| V66 | | SRRAQDFVQWLMNTLAPV | | |
| ITS011- | 3-20 | CARAESAG [symbol] HSQGTFTSDYSKYL | 1 | 39 |
| V69 | | DSRRAQDFVQWLMNTTSMVR | | |

Oxyntomodulin

| | | | | |
|---|---|---|---|---|
| ITS011- | 3-30 | CASN [symbol] HSQGTFTSDYSKYLDSRR | 6 | 40 |
| V23 | | AQDFVQWLMNTKRNRNNIAMLL | | |
| ITS011- | 3-20 | CARDKGI [symbol] HSQGTFTSDYSKYLD | 2 | 41 |
| V74 | | SRRAQDFVQWLMNTKRNRNNIAS | | |
| | | KQVR | | |
| ITS011- | 3-30 | CARDLSDLSHSVLL [symbol] HSQGTFTS | 1 | 42 |
| V100 | | DYSKYLDSRRAQDFVQWLMNTKR | | |
| | | NRNNIARPLVLS | | |
| ITS011- | 4-34 | CARGPILRPR [symbol] HSQGTFTSDYSK | 4 | 113 |
| V165 | | YLDSRRAQDFVQWLMNTKRNRNN | | |
| | | IAHSIV | | |
| ITS011- | 3-30 | CARDRFSRSFS [symbol] HSQGTFTSDYS | 4 or 5 | 114 |
| V171 | | KYLDSRRAQDFVQWLMNTKRNRN | | |
| | | NIAN | | |

*Peptide sequence in bold; charged amino acid flanking terminal Histidine of peptide sequence in underlined bold italics; carboxy flanking sequences found between peptide and JH gene segment in italics.

### DISCUSSION

Many in the field believed that it would not be possible to generate a functional peptide-grafted antibody to either the GLP1 receptor or the glucagon receptor because of the perceived requirement for a free amino terminal amino acid. The use of the V(D)J system to generate large de novo libraries as well as the ability to couple those large repertoires with a single cell functional read out has allowed rare functional antibodies to be identified. Unexpectedly, these rare solutions for the two receptors using three different peptides all showed a requirement for a positively charged amino acid adjacent to the natural N-terminal amino acid. The presence of the arginine or lysine appears to substitute for the free amino group in the natural peptide. The presence of the charged amino acid alone, however, may not be sufficient to provide a functional agonist as other peptide-grafted antibodies having an arginine in the −1 position were identified that did not show agonist activity in the assays used in these experiments. The presence of the arginine or lysine does seem to be required as no agonist antibodies have been identified to date that did not have either a lysine or arginine in this position.

It is worth noting that both glucagon and GLP1 are generated by cleavage of a proprotein that is inactive until it is processed and that the natural amino acid in the proprotein upstream of the N-terminal histidine is an arginine.

### Example 7

### Mutagenesis of N-Terminal Amino Acid Adjacent to Histidine in the GLP-1 HCAb Agonist V125

As described in Example 5, the original fully human peptide-grafted antibody ITS007-V107 was converted to a heavy chain only antibody HCAb format to generate an agonist HCAb, ITS007-V125 (Table 7 below, FIG. 3). The VH domains of the two agonists V107 and V125 are identical and both molecules are functional as agonists of the GLP-1 receptor. Mutagenesis of the amino acid upstream of natural N-terminal histidine of the Exendin sequence in the V125 peptide-grafted agonist HCAb was conducted. Specifically, variants of the V125 peptide-grafted HCAb were generated so that the arginine flanking the histidine was mutated to every amino acid. While some mutations were found to be detrimental to expression, the only mutant to retain GLP-1 receptor agonist activity was when the arginine was mutated to a lysine.

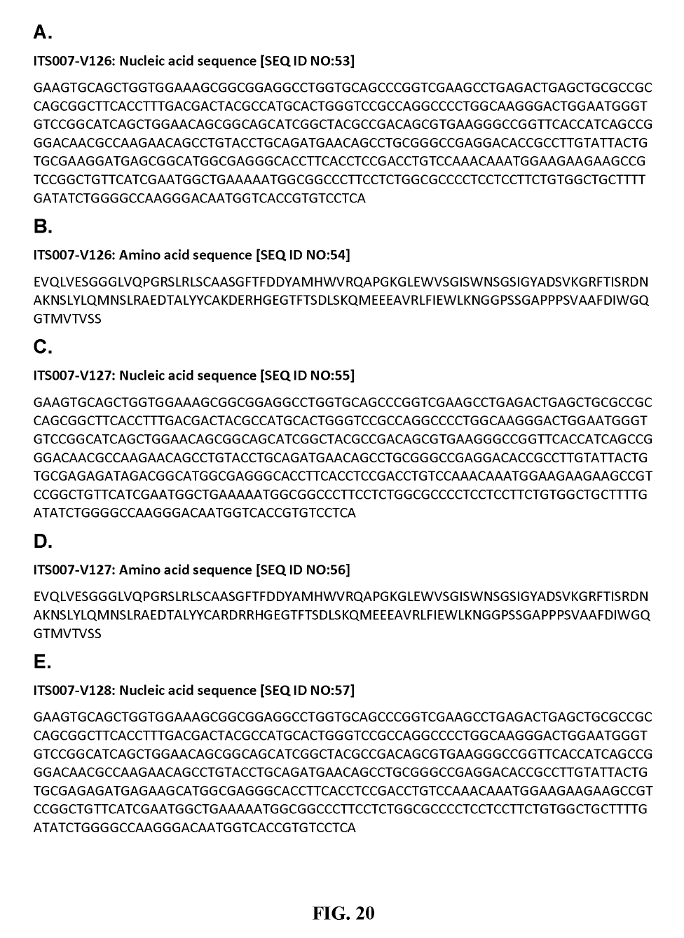

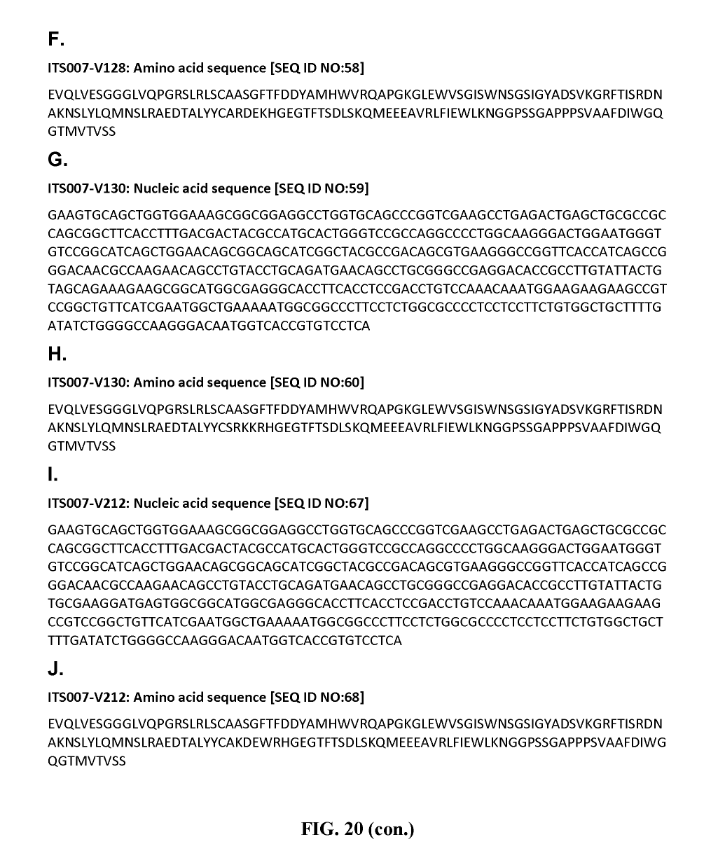

Once it had been determined that the agonist antibodies all contained a positive charge adjacent to the N-terminal histidine, additional site-specific mutants were generated using PCR to introduce additional positive charges at other positions between the VH and peptide (ITS007-V126, V127 and V128; see Table 7 and FIG. 20). An additional mutant ITS007-V130 (see Table 7 and FIG. 20) was also generated using PCR. In V130, the entire junction between the VH and the peptide includes basic amino acids. All of these modified peptide-grafted antibodies were shown to have agonist activity against the GLP-1 receptor.

An additional HCAb agonist with increased potency was also identified that had an insertion of a single amino-acid in the amino terminal VH-peptide junction, ITS007-V212 (see Table 7, and FIG. 20).

The observation that a flanking lysine also could support agonist activity of a grafted peptide antibody to a type 1 GPCR was corroborated with the subsequent isolation of glucagon HCAb agonists generated de novo from V(D)J peptide grafted substrates that contained a lysine flanking the histidine of the grafted glucagon peptide and oxyntomodulin peptide (ITS011-V066 and ITS011-V074, see Table 6 above).

It should be appreciated that the presence of a lysine or arginine N-terminally to the histidine of peptides to either the GLP-1 receptor or Glucagon receptor is not sufficient to impart agonist activity. In addition to non-agonist peptide-grafted HCAbs being identified with either a lysine or arginine in this position, when the Exendin peptide in another HCAb GLP1 agonist, ITS007-V126 (see Table 7, FIG. 20; SEQ ID NOs:53 and 54), was replaced with the glucagon peptide sequence, no glucagon activity was observed implying that the conformational context is important in positioning the charged amino acid so that it can contribute to generating agonist activity from the grafted peptide.

### Example 8

### CDR3 Optimization of HCAb Agonists

In order to improve the potency of the GLP-1 receptor agonists described above, V(D)J protein optimization as described in International Patent Application PCT/CA2013/050203 (WO 2013/134880) was applied to these HCAbs. Briefly, V(D)J substrates (a total of 15) were designed to target every other amino acid in CDR3 of the HCAb GLP1 agonists, V125, V126 and V130 (see Table 7, and FIG. 20), and used to generate membrane-bound HCAb variant libraries. Cells were selected with increased affinity to the amino-terminus of the GLP-1 receptor (FIG. 23A).

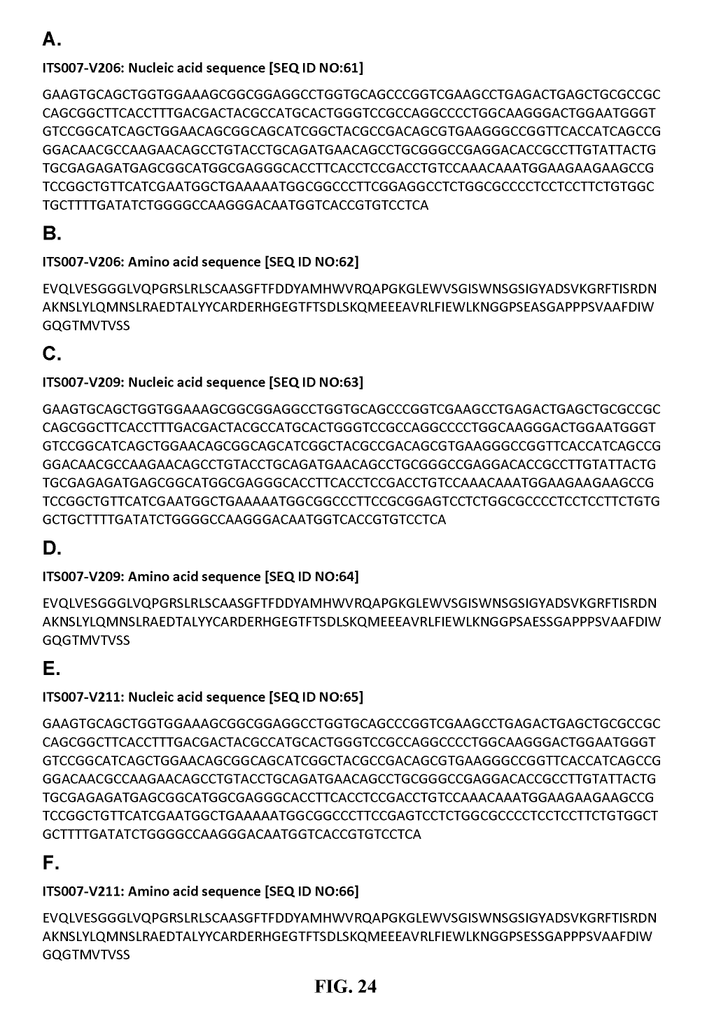

Several agonists with increased potency with mutations that clustered in the carboxy terminal region of the grafted peptide were identified. Three of these clones (ITS007-V206, -V209 and -V211; see Table 7 and FIG. 24) were shown to have approximately a 3-4 fold increase in agonist activity to the receptor (FIG. 23B), demonstrating that sequences outside of the amino terminus of the peptide derived sequences can modulate agonist activity. Interestingly, although each of the three sequences (V206, V209 and V211) was unique, they all shared a common amino acid change (see FIG. 24). Two of the sequences (ITS007-V209 and ITS007-V211) shared two amino acid changes.

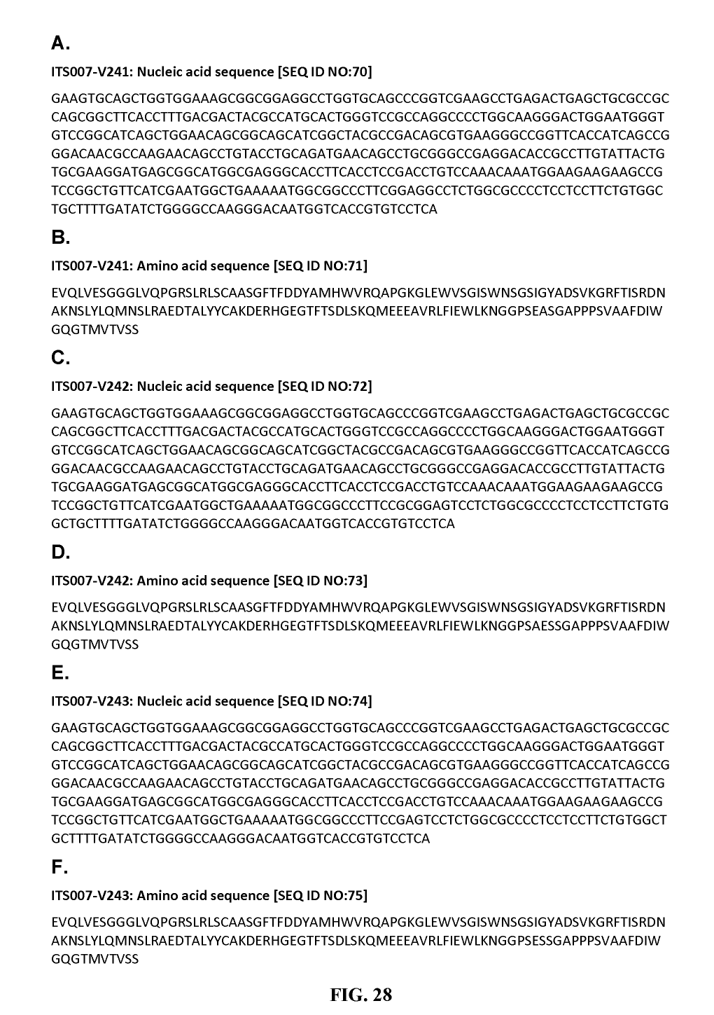

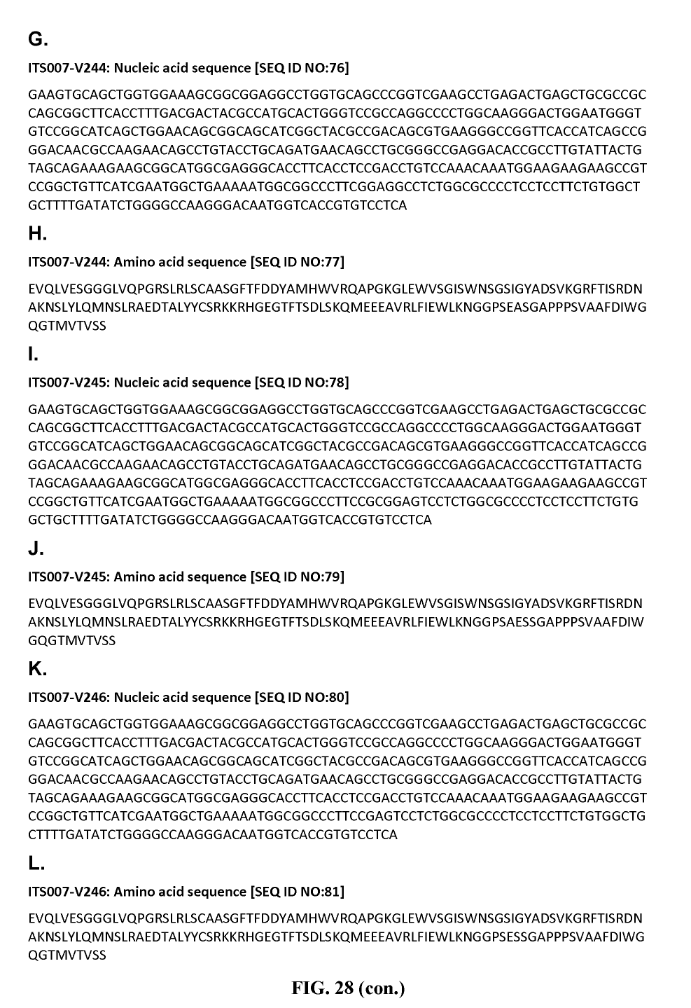

ITS007-V206, -V209 and -V211 were all generated in the context of the V125 amino terminal sequences. In a further experiment, these three different carboxy mutations were paired with V126 and V130 amino terminal sequences to provide to provide hybrid molecules ITS007-V241-V246 (Table 7, and FIG. 28). All six of these peptide-grafted HCAbs were shown to have agonist activity.

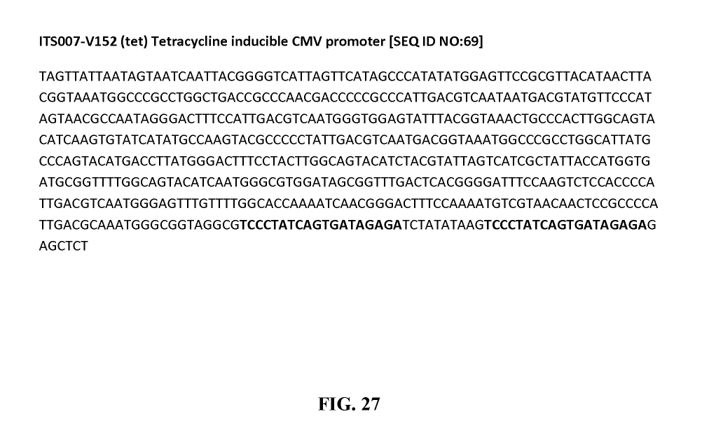

In a separate experiment, V(D)J mutagenesis was applied and agonist activity selected for directly. In order to be able to identify antibodies with increased potency, these V(D)J substrates were engineered with a tetracycline inducible TK promoter driving HCAb expression. This allowed the HCAb to be expressed at low levels and modulated with the addition of Tetracycline (ITS007-V152 (tet); see FIG. 27). In conditions with no tetracycline added to the culture, the agonist activity of cells constitutively expressing the V125 HCAb was below detection.

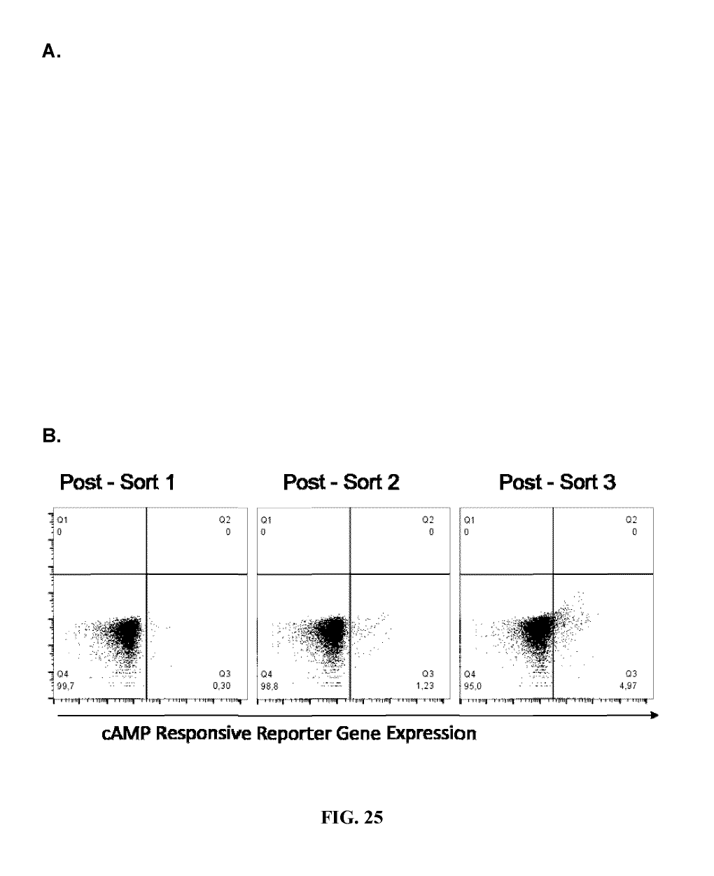

A V(D)J de novo generated library of approximately 10⁸ CDR3 variants targeting every other amino acid in CDR3 was magnetically enriched for CD19 positive cells and subsequently FACS sorted. Initial frequencies of CD19 positive cells were below detection (less than one in hundred thousand). Following magnetic enrichment via the CD19 surface marker of GLP-1 receptor activity, frequencies of approximately 0.5% were observed. Subsequent FACS sorts increased the frequency of CD19 positive cells to 14% representing cells expressing variants of ITS007-V125 with increased potency (FIG. 25A).

TABLE 7
Variant HCAb GLP1 Agonists

| | | | | |
|---|---|---|---|---|
| | | | | SEQ |
| | VH | | JH | ID |
| ID | Gene | Sequence* | Gene | NO |

Original clone in HcAb format

| | | | | |
|---|---|---|---|---|
| ITS007- | 3-9 | CARDE [symbol] HGEGTFTSDLSKQMEEEA | 3 | 82 |
| V125 | | VRLFIEWLKNGGPSSEAPPPSVA | | |

V-exendin junction mutants

| | | | | |
|---|---|---|---|---|
| ITS007- | 3-9 | CAKDE [symbol] HGEGTFTSDLSKQMEEEA | 3 | 83 |
| V126 | | VRLFIEWLKNGGPSSEAPPPSVA | | |
| ITS007- | 3-9 | CARDR [symbol] HGEGTFTSDLSKQMEEEA | 3 | 84 |
| V127 | | VRLFIEWLKNGGPSSEAPPPSVA | | |
| ITS007- | 3-9 | CARDE [symbol] HGEGTFTSDLSKQMEEEA | 3 | 85 |
| V128 | | VRLFIEWLKNGGPSSEAPPPSVA | | |
| ITS007- | 3-9 | CSRKK [symbol] HGEGTFTSDLSKQMEEEA | 3 | 86 |
| V130 | | VRLFIEWLKNGGPSSAPPPSVA | | |
| ITS007- | 3-9 | CAKDE [symbol] HGEGTFTSDLSKQMEEE | 3 | 87 |
| V212 | | AVRLFIEWLKNGGPSSGAPPPSVA | | |

C-terminal exendin mutants

| | | | | |
|---|---|---|---|---|
| ITS007- | 3-9 | CARDE [symbol] HGEGTFTSDLSKQMEEEAV | 3 | 88 |
| V206 | | RLFIEWLKNGGPSEASGAPPPSVA | | |
| ITS007- | 3-9 | CARDE [symbol] HGEGTFTSDLSKQMEEEAV | 3 | 89 |
| V209 | | RLFIEWLKNGGPSAESSGAPPPSVA | | |
| ITS007- | 3-9 | CARDE [symbol] HGEGTFTSDLSKQMEEEAV | 3 | 90 |
| V211 | | RLFIEWLKNGGPSESSGAPPPSVA | | |

Hybrid mutants†

| | | | | |
|---|---|---|---|---|
| ITS007- | 3-9 | CAKDE [symbol] HGEGTFTSDLSKQMEEEAV | 3 | 91 |
| V241 | | RLFIEWLKNGGPSEASGAPPPSVA | | |
| ITS007- | 3-9 | CAKDE [symbol] HGEGTFTSDLSKQMEEEAV | 3 | 92 |
| V242 | | RLFIEWLKNGGPSAESSGAPPPSVA | | |
| ITS007- | 3-9 | CAKDE [symbol] HGEGTFTSDLSKQMEEEAV | 3 | 93 |
| V243 | | RLFIEWLKNGGPSESSGAPPPSVA | | |
| ITS007- | 3-9 | CSRKK [symbol] HGEGTFTSDLSKQMEEEAV | 3 | 94 |
| V244 | | RLFIEWLKNGGPSEASGAPPPSVA | | |
| ITS007- | 3-9 | CSRKK [symbol] HGEGTFTSDLSKQMEEEAV | 3 | 95 |
| V245 | | RLFIEWLKNGGPSAESSGAPPPSVA | | |
| ITS007- | 3-9 | CSRKK [symbol] HGEGTFTSDLSKQMEEEAV | 3 | 96 |
| V246 | | RLFIEWLKNGGPSESSGAPPPSVA | | |

*Peptide sequence in bold; charged amino acid flanking terminal Histidine of peptide sequence in underlined bold italics; carboxy flanking sequences found between peptide and JH gene segment in italics; additional mutations of interest underlined.
†based on: V241: ITS007-V126/ITS007-V206; V242: ITS007-V126/ITS007-V209; V243: ITS007-V126/ITS007-V211; V244: ITS007-V130/ITS007-V206; V245: ITS007-V130/ITS007-V209; V246: ITS007-V130/ITS007-V211.

### Example 9

### CDR1 and CDR2 Optimization of HCAb Agonists

A V(D)J de novo generated library of approximately 10⁸ CDR1 and CDR2 variants was also generated. Initial frequencies of CD19 positive cells were again below detection (less than one in hundred thousand). Following magnetic enrichment via the CD19 surface marker (expressed following activation of GLP-1 receptor), frequencies of approximately 0.3% were observed. Subsequent FACS sorts increased the frequency of CD19 positive cells to approximately 4% for the CDR1 and CDR2 variant pools (FIG. 25B). This latter result demonstrates the potential to increase the potency of HCAb agonists by mutagenesis outside of the peptide sequences and of CDR3.

### Example 10

### Increasing Potency of HCAbs by Addition of a VH Domain

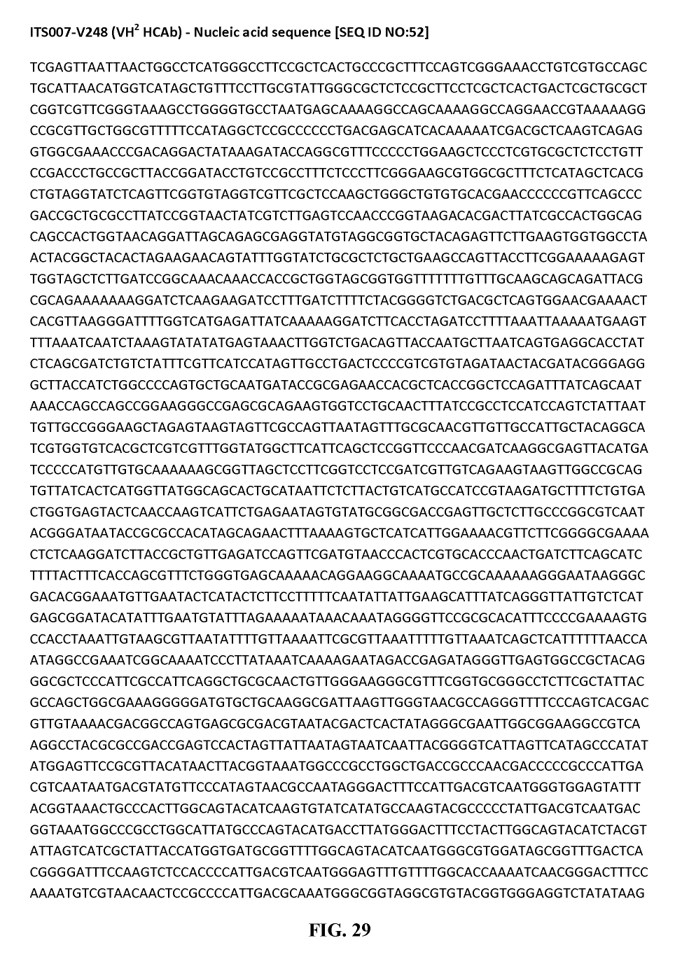

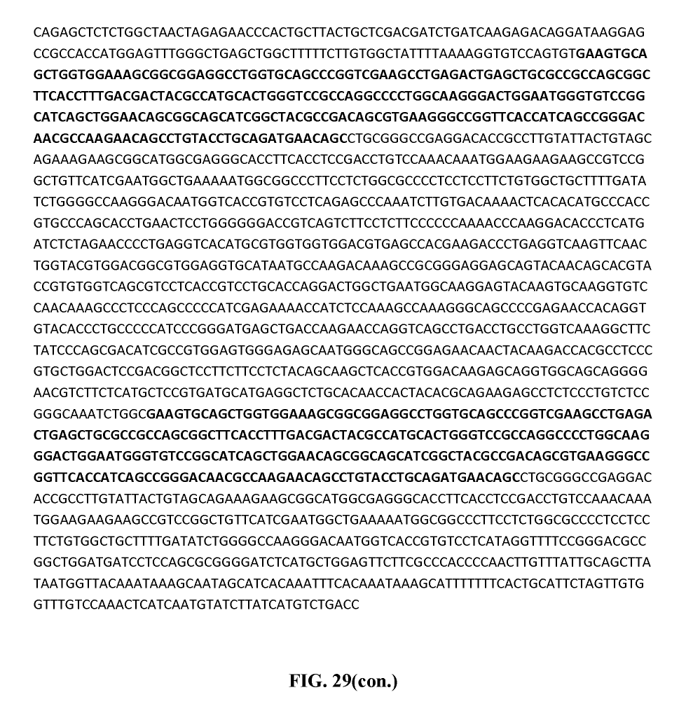

In order to investigate whether the potency of the GLP-1 agonist HCAbs described above could be increased, a construct that placed the GLP-1R agonist V130 VH domain downstream of the CH3 sequences of the V130 HCAb was prepared (ITS007-V248, see FIG. 29). The ITS007-V248 construct was generated by using PCR to amplify the VH domain and place it downstream of the CH3 domain of the ITS007-V130 HCAb. A schematic of this molecule, which contains a total of 4 VH agonist domains, is shown in FIG. 22B. This format of HCAb is referred to herein as “VH².”

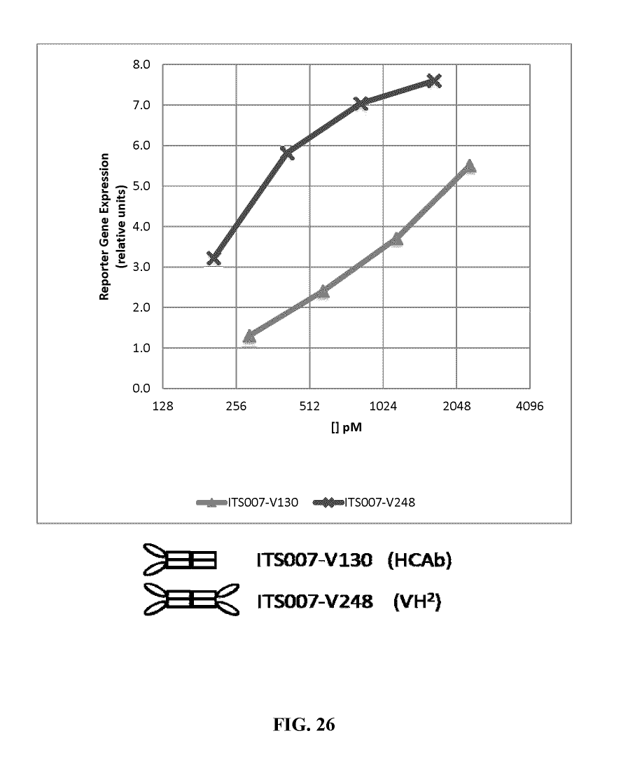

Transient transfections were performed and supernatant was generated and quantitated for IgG. A titration of the supernatants was performed assaying for agonist activity, and activity was compared to supernatants generated from the original V130 HCAb. As can be seen in FIG. 26, not only is the VH² active but it appears to be approximately 4 times more potent than the original V130 HCAb. This increase represents more than just a doubling, which would be expected from the presence of the two additional VH domains.

This example not only confirms that the VH domain is modular, but also provides a method to enhance the potency of an existing HCAb or to generate molecules with multiple functionality. For example, placing a GLP-1 receptor agonist VH domain on a glucagon agonist HCAb could generate a dual agonist HCAb. Likewise, placing a VH domain downstream of a full antibody would allow the generation of a novel bi-specific antibody that could potentially target GLP-1 agonist activity if desired.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

The embodiments of the invention in which an exclusive property or privilege is claimed are defined as follows:

1. A peptide-grafted antibody or antibody fragment comprising at least one peptide grafted into at least one CDR of the antibody or antibody fragment, the peptide comprising the sequence of a peptide agonist of a Class B GPCR or a functional fragment of the peptide agonist, wherein the peptide-grafted antibody has agonist activity against the Class B GPCR.

2. The peptide-grafted antibody or antibody fragment according to claim 1, wherein the Class B GPCR is a Subfamily B1 GPCR.

3. The peptide-grafted antibody or antibody fragment according to claim 2, wherein the Subfamily B1 GPCR is the PACAP type 1 receptor, CALCR, CRHR, GLP1 receptor, GLP2 receptor, glucagon receptor, SCTR or VIP receptor.

4. The peptide-grafted antibody or antibody fragment according to claim 3, wherein the peptide agonist is exendin, GLP1, GLP2, glucagon, oxyntomodulin, PACAP-27, PACAP-38, VIP, helodermin, PHM or secretin.

5. The peptide-grafted antibody or antibody fragment according to any one of claims 1 to 4, wherein the grafted peptide comprises a basic amino acid upstream and adjacent to the natural N-terminal amino acid of the peptide agonist.

6. The peptide-grafted antibody or antibody fragment according to claim 5, wherein the basic amino acid is an arginine or a lysine.

7. A peptide-grafted antibody or antibody fragment comprising at least one peptide grafted into at least one CDR of the antibody or antibody fragment, the peptide comprising the sequence of the exendin, GLP1, glucagon or oxyntomodulin peptide or a functional fragment thereof, wherein the peptide-grafted antibody has agonist activity against the GLP1 receptor, the glucagon receptor, or both.

8. The peptide-grafted antibody or antibody fragment according to claim 7, wherein the grafted peptide comprises a basic amino acid upstream and adjacent to the natural N-terminal histidine of the exendin, GLP1, glucagon or oxyntomodulin peptide.

9. The peptide-grafted antibody or antibody fragment according to claim 8, wherein the basic amino acid is an arginine or a lysine.

10. The peptide-grafted antibody or antibody fragment according to any one of claims 5, 6, 8 or 9, wherein the basic amino acid is comprised by a heterologous flanking sequence of between one and about 20 amino acids at the N-terminus of the grafted peptide.

11. The peptide-grafted antibody or antibody fragment according to any one of claims 5, 6, 8 or 9, wherein the basic amino acid is part of the antibody sequence N-terminal to the grafted peptide.

12. The peptide-grafted antibody or antibody fragment according to any one of claims 1 to 11, wherein the CDR is a heavy chain CDR.

13. The peptide-grafted antibody or antibody fragment according to any one of claims 1 to 11, wherein the CDR is a light chain CDR.

14. The peptide-grafted antibody or antibody fragment according to any one of claims 1 to 13, wherein the CDR is a CDR3.

15. The peptide-grafted antibody or antibody fragment according to any one of claims 1 to 14, further comprising a heterologous flanking sequence of between one and about 20 amino acids at the C-terminus of the grafted peptide.

16. The peptide-grafted antibody according to any one of claims 7 to 15, wherein the antibody is a heavy-chain only antibody.

17. The peptide-grafted antibody fragment according to any one of claims 7 to 15, wherein the antibody fragment is a single VH domain antibody.

18. The peptide-grafted antibody according to claim 16, further comprising a peptide-grafted VH domain attached to a CH3 region of the peptide-grafted antibody, wherein the peptide comprised by the peptide-grafted VH domain comprises the sequence of the exendin, GLP1, glucagon or oxyntomodulin peptide or a functional fragment thereof.

19. The peptide-grafted antibody according to claim 18, wherein the peptide comprised by peptide-grafted VH domain and the peptide comprised by the peptide-grafted antibody target the same receptor.

20. The peptide-grafted antibody according to claim 18, wherein the peptide comprised by peptide-grafted VH domain and the peptide comprised by the peptide-grafted antibody target different receptors.

21. The peptide-grafted antibody or antibody fragment according to any one of claims 7 to 20, wherein the peptide comprised by the peptide-grafted antibody comprises the sequence of the exendin or GLP1 peptide, or a functional fragment thereof, and the peptide-grafted antibody has agonist activity against the GLP1 receptor.

22. The peptide-grafted antibody or antibody fragment according to claim 21, wherein the peptide-grafted antibody comprises the sequence as set forth in any one of SEQ ID NOs: 32, 33 and 82 to 96.

23. The peptide-grafted antibody or antibody fragment according to claim 21, wherein the peptide-grafted antibody comprises the sequence as set forth in any one of SEQ ID NOs:24, 26, 51, 54, 56, 58, 60, 62, 64, 66, 68, 71, 73, 75, 77, 79 or 81.

24. The peptide-grafted antibody or antibody fragment according to any one of claims 7 to 20, wherein the peptide peptide-grafted antibody comprises the sequence of the glucagon or oxyntomodulin peptide, or a functional fragment thereof, and the peptide-grafted antibody has agonist activity against the glucagon receptor.

25. The peptide-grafted antibody or antibody fragment according to claim 24, wherein the peptide-grafted antibody comprises the sequence as set forth in any one of SEQ ID NOs: 37 to 42, 113 and 114.

26. The peptide-grafted antibody or antibody fragment according to claim 24, wherein the peptide-grafted antibody comprises the sequence as set forth in any one of SEQ ID NOs:98, 100, 102, 104, 106, 108, 110 or 112.

27. A polynucleotide encoding the peptide-grafted antibody or antibody fragment according to any one of claims 1 to 27.

28. A mammalian cell line for high-throughput screening for agonists of a Class B GPCR, the cell line comprising:
a first stably integrated recombinant nucleic acid construct comprising a reporter gene operably associated with a cAMP responsive promoter, the reporter gene encoding a cell-surface expressed protein, and
a second stably integrated recombinant nucleic acid construct comprising a sequence encoding a Class B GPCR operably associated with a promoter.

29. The cell line according to claim 28, wherein the cell line is recombination competent.

30. The cell line according to claim 29, wherein the cell line comprises polynucleotides encoding RAG-1 and RAG-2.

31. The cell line according to any one of claims 28 to 30, further comprising one or more sites engineered into the chromosome that permit stable chromosomal integration of a heterologous sequence.

32. The cell line according to claim 31, wherein the heterologous sequence is a nucleic acid sequence encoding a plurality of VH gene sequences, a candidate peptide agonist of the Class B GPCR and at least one JH gene sequence, and further comprising one or more pairs of RSS sequences positioned to allow recombination of the nucleic acid sequence to form a polynucleotide encoding an antibody heavy chain comprising the peptide agonist.

33. The cell line according to claim 31, wherein the heterologous sequence is a nucleic acid sequence encoding a plurality of VL gene sequences, a candidate peptide agonist of the Class B GPCR and at least one JL gene sequence, and further comprising one or more pairs of RSS sequences positioned to allow recombination of the nucleic acid sequence to form a polynucleotide encoding an antibody light chain comprising the peptide agonist.

34. The cell line according to any one of claims 28 to 33, wherein the reporter gene encodes CD19.

35. The cell line according to any one of claims 28 to 34, wherein the Class B GPCR is the GLP1 receptor or the glucagon receptor.

36. The cell line according to any one of claims 28 to 35, wherein the cell line is a human cell line.

37. A method for screening for peptide-grafted antibodies having agonist activity against a Class B GPCR, the method comprising:
stably transfecting cells from the cell line of claim 27 with a library of polynucleotides encoding peptide-grafted antibodies, wherein the peptide is a candidate peptide agonist of a Class B GPCR;
culturing the cells under conditions that permit expression of the peptide-grafted antibodies and expression of the Class B GPCR, and
screening the cells for expression of the cell-surface expressed protein.

38. A method for screening for peptide-grafted antibodies having agonist activity against a Class B GPCR, the method comprising:
stably transfecting cells from the cell line of an claim 28 or 29 with either (a) a nucleic acid sequence encoding a plurality of VH gene sequences, a candidate peptide agonist of the Class B GPCR and at least one JH gene sequence, and comprising one or more pairs of RSS sequences positioned to allow recombination of the nucleic acid sequence to form a polynucleotide encoding an antibody heavy chain comprising the peptide agonist, or (b) a nucleic acid sequence encoding a plurality of VL gene sequences, a candidate peptide agonist of the Class B GPCR and at least one JL gene sequence, and comprising one or more pairs of RSS sequences positioned to allow recombination of the nucleic acid sequence to form a polynucleotide encoding an antibody light chain comprising the peptide agonist;
culturing the cells under conditions that permit V(D)J recombination, expression of the antibody heavy or light chain comprising the peptide agonist and expression of the Class B GPCR, and
screening the cultured cells for expression of the cell-surface expressed protein.

39. The method according to claim 37 or 38, wherein the polynucleotides encoding the peptide-grafted antibodies or the nucleic acid sequence further comprise a sequence encoding a plasma anchor domain.

40. The method according to any one of claims 37 to 39, wherein the cell line further comprises one or more sites engineered into the chromosome that permit stable chromosomal integration of a heterologous sequence.

41. The method according to claim 40, wherein in the step of stably transfecting, the polynucleotides or nucleic acid sequence is stably integrated into the chromosome at the one or more sites.

42. The method according to any one of claims 37 to 41, wherein the reporter gene encodes CD19.

43. The method according to any one of claims 37 to 42, wherein the Class B GPCR is the GLP1 receptor or the glucagon receptor.

44. The method according to any one of claims 37 to 43, wherein the cell line is a human cell line.

45. The method according to any one of claims 37 to 44, further comprising isolating a cell that expresses the cell-surface expressed protein.

46. The method according to claim 45, further comprising isolating a polynucleotide encoding the peptide-grafted antibody having agonist activity against a Class B GPCR from the isolated cell.

47. A peptide-grafted antibody agonist encoded by the polynucleotide obtained by the method according to claim 46.

48. A method for preparing a heavy-chain only peptide-grafted antibody having agonist activity against a Class B GPCR, the method comprising:
stably transfecting cells from the cell line of an claim 28 or 29 with a nucleic acid sequence encoding a plurality of VH gene sequences, a candidate peptide agonist of the Class B GPCR and at least one JH gene sequence, and comprising one or more pairs of RSS sequences positioned to allow recombination of the nucleic acid sequence to form a polynucleotide encoding an antibody heavy chain comprising the peptide agonist;
culturing the cells under conditions that permit V(D)J recombination, expression of the antibody heavy chain comprising the peptide agonist and expression of the Class B GPCR;
screening the cultured cells for expression of the cell-surface expressed protein;
isolating a cell that expresses the cell-surface expressed protein, and
isolating a polynucleotide encoding the peptide-grafted antibody having agonist activity against a Class B GPCR from the isolated cell.
